(19) United States
(12) Patent Application Publication
(10) Pub. No.: US 2024/0067655 A1
(43) Pub. Date: Feb. 29, 2024

(54) KINASE MODULATORS AND METHODS OF USE THEREOF

(71) Applicant: NEURON23, INC., South San Francisco, CA (US)

(72) Inventors: Jean Tassel, Augsburg (DE); Michael Thormann, Planegg (DE); Roland Koestler, Martinsried (DE); Andreas Treml, Bodenmais (DE); Zhonghua Pei, South San Francisco, CA (US); Ming Yu, South San Francisco, CA (US)

(21) Appl. No.: 18/036,549

(22) PCT Filed: Nov. 15, 2021
(86) PCT No.: PCT/US2021/059378

§ 371 Date: May 11, 2023

Related U.S. Application Data
(60) Provisional application No. 63/113,523, filed on Nov. 13, 2020.
(60) Provisional application No. 63/113,533, filed on Nov. 13, 2020.

(51) Int. Cl. C07D 487/16; C07D 487/22; C07D 471/22

(52) U.S. Cl. CPC C07D 487/16; C07D 487/22; C07D 471/22

(57) ABSTRACT

The present invention relates to novel compounds that are capable of modulating, e.g., inhibiting or activating, one or more kinases, especially LRRK2 and/or NUAK1 and/or TYK2 or mutants thereof. The compounds are useful for treating diseases, such as autoimmune diseases, inflammatory diseases, bone diseases, metabolic diseases, neurological and neurodegenerative diseases, cancer, cardiovascular diseases, allergies, asthma, Alzheimer's disease, Parkinson's disease, skin disorders, eye diseases, infectious diseases and hormone-related diseases.

### RELATED APPLICATIONS

The present application claims the benefit of and priority to each of U.S. provisional patent application Ser. No. 63/113,523, filed Nov. 13, 2020, and U.S. provisional patent application Ser. No. 63/113,533, filed Nov. 13, 2020, the content of each of which is incorporated by reference herein in its entirety.

### FIELD OF THE INVENTION

The invention relates generally to compounds and their use in pharmacological composition for treatment of conditions as well as radio-labeled tracers in positron emission tomography (PET) for diagnostic uses.

### BACKGROUND

A variety of medical conditions that affect millions of people are caused or exacerbated by unregulated activity of protein kinases. For example, aberrant kinase activity is associated with autoimmune diseases, inflammatory diseases, bone diseases, metabolic diseases, neurological and neurodegenerative diseases, cancer, cardiovascular diseases, allergies, asthma, Alzheimer's disease, Parkinson's disease, skin disorders, eye diseases, infectious diseases and hormone-related diseases. For many such disorders, however, no effective inhibitor or activator exists for the particular kinase that causes the disorder or its symptoms. Consequently, patients continue to suffer from an array of disorders due to the lack of a suitable drug for their conditions.

### SUMMARY

The invention provides compounds that are useful in pharmacological composition for treatment of conditions as well as radio-labeled tracers in positron emission tomography (PET) for diagnostic uses. In certain embodiments, the compounds of the invention modulate, e.g., inhibit or activate, inhibit protein kinase activity, such as the activity of leucine-rich repeat kinase 2 (LRRK2), SNF1-like kinase 1 also known as AMPK-related protein kinase 5 (NUAK1) also known as (ARK5), and non-receptor tyrosine-protein kinase 2 (TYK2), that are associated with human diseases, disorders, and conditions. The compounds display improved pharmacological properties, such as tissue delivery, specificity, efficacy, and stability. For example, the invention includes compounds that are able to penetrate the blood-brain barrier and bind to kinase targets with high affinity. Additionally, radiolabeled forms of compounds of the invention are useful as PET tracers to identify anatomical locations of aberrant kinase activity.

Thus, compounds of the invention are useful as therapeutic and diagnostic agents for a wide variety of conditions, such as Parkinson's disease and autoimmune diseases.

Accordingly, the invention provides compositions containing compounds described herein, including pharmacological compositions and compositions for diagnostic applications. The invention further provides methods of using such compositions to diagnose and/or treat a disorder in a subject.

In an aspect, the invention provides compounds of formula (I):

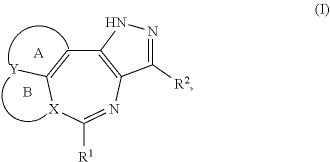

or a tautomer thereof, or a pharmaceutically acceptable salt thereof,
wherein:
R¹ is mono- or bicyclic aryl or heteroaryl;
R² is H, halo, OH, CN, CF₃, C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, or heteroaryl;
A is aryl, heteroaryl, cycloalkyl, heterocycloalkyl, cycloalkenyl, or heterocycloalkenyl;
B is aryl, heteroaryl, cycloalkyl, heterocycloalkyl, cycloalkenyl, or heterocycloalkenyl;
X is C or N; and
Y is C or N,
   wherein each of the substituents in each of R¹, R², A, and B is independently optionally substituted with one or more moieties selected from the group consisting of halo, OH, CN, CF₃, NH₂, NO₂, C₁₋₆ alkyl, C₁₋₆ haloalkyl, C₁₋₆ thioalkyl, C₃₋₈ cycloalkyl, C₂₋₈ heterocycloalkyl, C₂₋₈ heterocycloalkenyl, C₂₋₆ alkenyl, C₂₋₆ alkynyl, C₁₋₆ alkoxy, C₁₋₆ haloalkoxy, C₁₋₆ alkylamino, C₂₋₆ dialkylamino, C₇₋₁₂ aralkyl, C₁₋₁₂ heteroaralkyl, aryl, heteroaryl, —C(O)R, —C(O)OR, —C(O)NRR′, —C(O)NRS(O)₂R′, —C(O)NRS(O)₂NR′R″, —OR, —OC(O)NRR′, —NRR′, —NRC(O)R′, —NRC(O)NR′R″, —NRS(O)₂R′, —NRS(O)₂NR′R″, —S(O)₂R, and —S(O)₂NRR′,
in which each of R, R′, and R″, independently, is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl, or R and R′, or R′ and R″, together with the nitrogen to which they are attached, form C₂₋₈ heterocycloalkyl.

Each of A and B may independently have a defined number of atoms. For example, each of A and B may independently have 4-7 atoms, 5-7 atoms, 6-7 atoms, 4-6 atoms, 5-6 atoms, or 4-5 atoms.

R¹ may be an optionally substituted 6-membered aryl or heteroaryl, or may be an optionally substituted 10-member bicyclic aryl or heteroaryl. The heteroaryl may be a pyridine. R¹ may be substituted with 1-3 of a -Me, ester (for example —OMe), halogen, alcohol (for example —OH or —COH), cyano, —CF₃, CHF₂, —OCHF₂, or cyclopropyl group.

R₁ may have the structure.

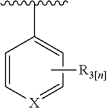

wherein X is carbon or nitrogen, and X may be in an ortho, meta, or para position on the ring,
where n is 0, 1, 2, or 3, and
wherein each R₃ may be independently selected from a -Me, ester (for example —OMe), halogen, alcohol (for example —OH or —COH), cyano, —CF₃, CHF₂, —OCHF₂, or cyclopropyl group.

The compound of formula (I) may have a structure selected from the group consisting of Compounds 301-319 or 340-408 and tautomers thereof:

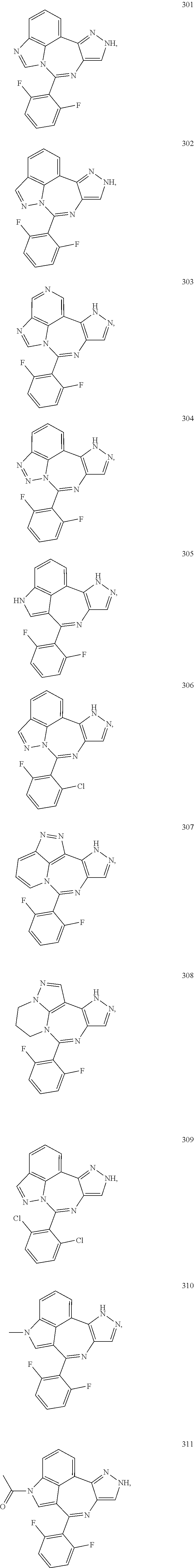

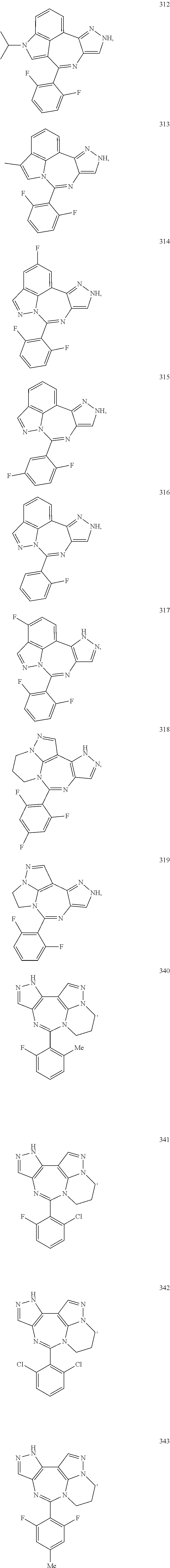

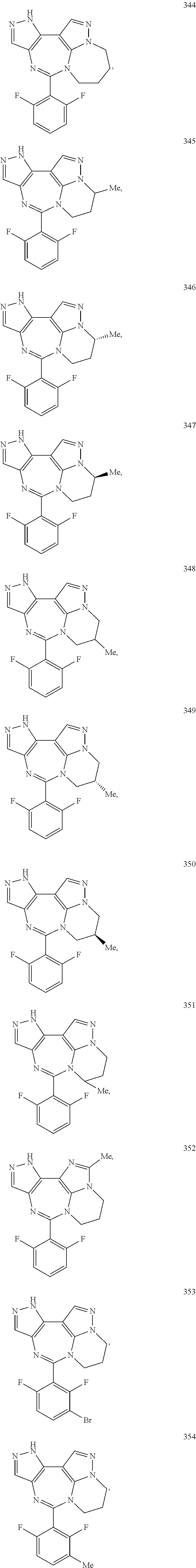

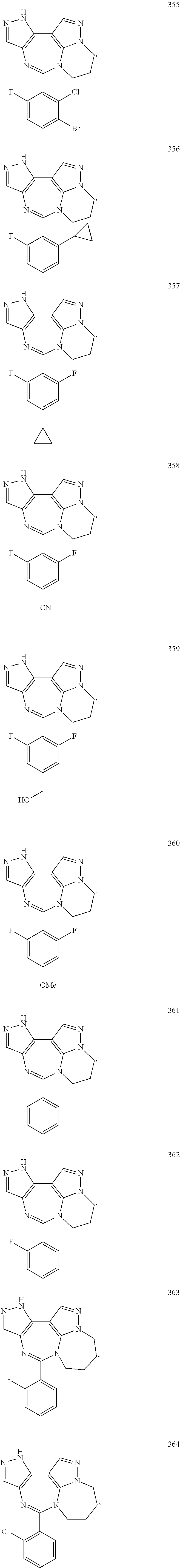

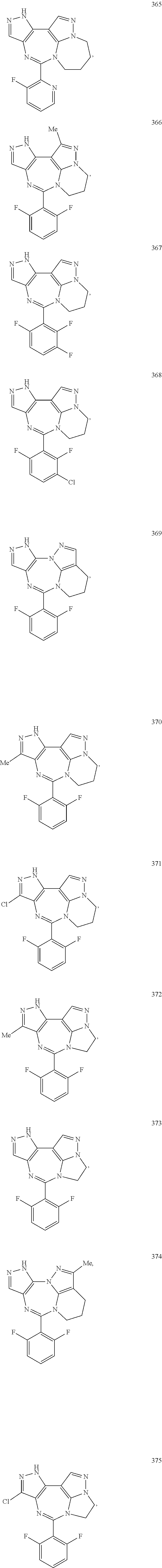

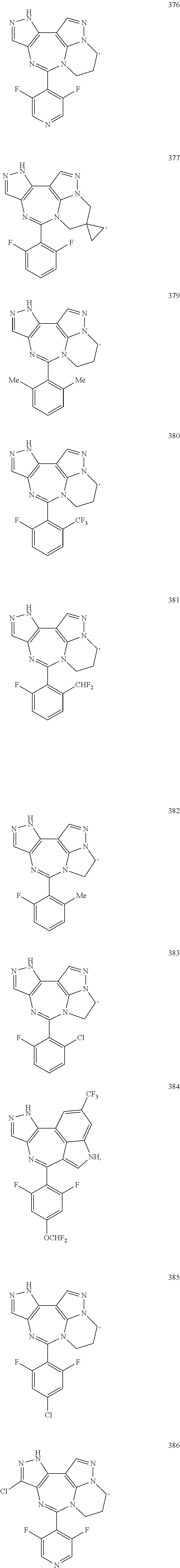

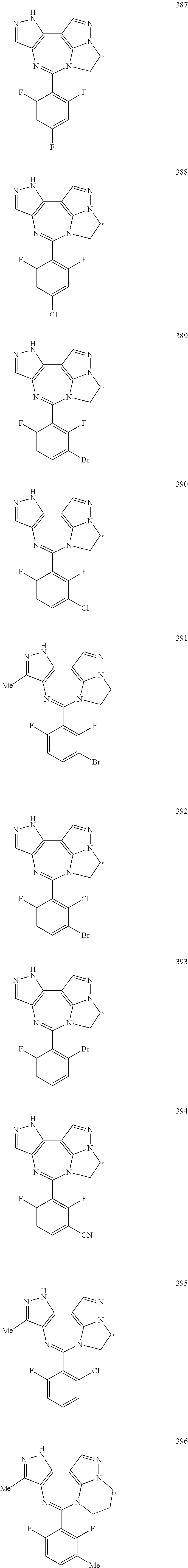

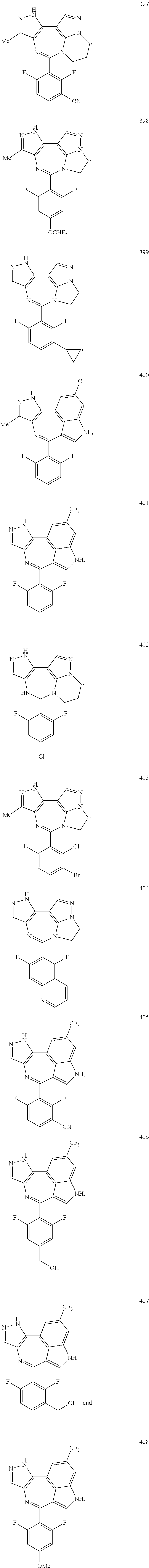

### DETAILED DESCRIPTION

### Chemical Definitions

The expression cycloalkyl refers to a saturated or partially unsaturated (for example, a cycloalkenyl group) cyclic group that contains one or more rings and contains from 3 to 14 ring carbon atoms, such as from 3 to 10 (e.g., 3, 4, 5, 6 or 7) ring carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH, N₃ or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbomyl, cyclohexyl, cyclopentenyl, cyclohexadienyl, decalinyl, bicyclo[4.3.0]nonyl, tetraline, cyclopentylcyclohexyl, fluorocyclohexyl or cyclohex-2-enyl group.

The expression heterocycloalkyl refers to a cycloalkyl group as defined above in which one or more (e.g., 1, 2 or 3) ring carbon atoms have been replaced by an oxygen, nitrogen, silicon, selenium, phosphorus or sulfur atom or a SO group or a SO₂ group. A heterocycloalkyl group has preferably 1 or 2 rings containing from 3 to 10 (e.g., 3, 4, 5, 6 or 7) ring atoms (e.g., C, O, N or S). The expression heterocycloalkyl refers furthermore to groups that are substituted by fluorine, chlorine, bromine or iodine atoms or by OH, ═O, SH, ═S, NH₂, ═NH, N₃ or NO₂ groups. Examples are a piperidyl, prolinyl, imidazolidinyl, piperazinyl, morpholinyl, urotro pinyl, pyrrolidinyl, tetrahydrothiophenyl, tetrahydropyranyl, tetrahydrofuryl or 2-pyrazolinyl group and also lactams, lactones, cyclic imides and cyclic anhydrides.

The expression alkylcycloalkyl refers to groups that contain both cycloalkyl and also alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two rings having from 3 to 10 (e.g., 3, 4, 5, 6 or 7) ring carbon atoms, and one or two alkyl, alkenyl or alkynyl groups having 1 or 2 to 6 carbon atoms.

The expression heteroalkylcycloalkyl refers to alkylcycloalkyl groups as defined above in which one or more (e.g., 1, 2 or 3) carbon atoms have been replaced by an oxygen, nitrogen, silicon, selenium, phosphorus or sulfur atom or a SO group or a SO₂ group. A heteroalkylcycloalkyl group preferably contains 1 or 2 rings having from 3 to 10 (e.g., 3, 4, 5, 6 or 7) ring atoms, and one or two alkyl, alkenyl, alkynyl or heteroalkyl groups having from 1 or 2 to 6 carbon atoms. Examples of such groups are alkyheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocycloalkenyl, the cyclic groups being saturated or mono-, di- or tri-unsaturated.

The expression aryl refers to an aromatic group that contains one or more rings containing from 6 to 14 ring carbon atoms, such as from 6 to 10 ring carbon atoms. The expression aryl refers furthermore to groups that are substituted by fluorine, chlorine, bromine or iodine atoms or by OH, SH, NH₂, N₃ or NO₂ groups. Examples are the phenyl, naphthyl, biphenyl, 2-fluorophenyl, anilinyl, 3-nitrophenyl or 4-hydroxyphenyl group.

The expression heteroaryl refers to an aromatic group that contains one or more rings containing from 5 to 14 ring atoms, such as from 5 to 10 ring atoms, and contains one or more (e.g., 1, 2, 3 or 4) oxygen, nitrogen, phosphorus or sulfur ring atoms. The expression heteroaryl refers furthermore to groups that are substituted by fluorine, chlorine, bromine or iodine atoms or by OH, SH, N₃, NH₂ or NO₂ groups. Examples are pyridyl (e.g. 4-pyridyl), imidazolyl (e.g. 2-imidazolyl), phenylpyrrolyl (e.g. 3-phenylpyrrolyl), thiazolyl, isothiazolyl, 1,2,3-triazolyl, 1,2,4-triazolyl, oxadiazolyl, thiadiazolyl, indolyl, indazolyl, tetrazolyl, pyrazinyl, pyrimidinyl, pyridazinyl, oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (e.g. 3-pyrazolyl) and isoquinolinyl groups.

The expression aralkyl refers to groups containing both aryl and also alkyl, alkenyl, alkynyl and/or cycloalkyl groups in accordance with the above definitions, such as, for example, aryl-alkyl, arylalkenyl, arylalkynyl, arylcycloalkyl, arylcycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, tetraline, dihydronaphthalene, indanone, phenylcyclopentyl, cumene, cyclohexylphenyl, fluorene and indane. An aralkyl group preferably contains one or two aromatic ring systems containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl refers to an aralkyl group as defined above in which one or more (e.g., 1, 2, 3 or 4) carbon atoms have been replaced by an oxygen, nitrogen, silicon, selenium, phosphorus, boron or sulfur atom, that is to say to groups containing both aryl or heteroaryl, respectively, and also alkyl, alkenyl, alkynyl and/or heteroalkyl and/or cycloalkyl and/or heterocycloalkyl groups in accordance with the above definitions. A heteroaralkyl group preferably contains one or two aromatic ring systems containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, wherein 1, 2, 3 or 4 of these carbon atoms have been replaced by oxygen, sulfur or nitrogen atoms.

Examples are arylheteroalkyl, arylheterocycloalkyl, arylheterocycloalkenyl, arylalkyl heterocycloalkyl, arylalkenylheterocycloalkyl, arylalkynylheterocycloalkyl, arylalkylhetero cycloalkenyl, heteroarylalkyl, heteroarylalkenyl, heteroarylalkynyl, heteroarylheteroalkyl, heteroarylcycloalkyl, heteroarylcycloalkenyl, heteroarylheterocycloalkyl, hetero arylheterocycloalkenyl, heteroarylalkylcycloalkyl, heteroarylalkylheterocycloalkenyl, hetero arylheteroalkylcycloalkyl, heteroarylheteroalkylcycloalkenyl and heteroarylheteroalkylhetero cycloalkyl groups, the cyclic groups being saturated or mono-, di- or tri-unsaturated. Specific examples are a tetrahydroisoquinolinyl, benzoyl, 2- or 3-ethylindolyl, 4-methylpyridino, 2-, 3- or 4-methoxyphenyl, 4-ethoxyphenyl, 2-, 3- or 4-carboxyphenylalkyl group.

As stated above, the expressions cycloalkyl, heterocycloalkyl, alkylcycloalkyl, heteroalkylcycloalkyl, aryl, heteroaryl, aralkyl and heteroaralkyl also refer to groups that are substituted by fluorine, chlorine, bromine or iodine atoms or by OH, ═O, SH, ═S, NH₂, ═NH, N₃ or NO₂ groups.

The expression “optionally substituted” especially refers to groups that are optionally substituted by fluorine, chlorine, bromine or iodine atoms or by OH, ═O, SH, ═S, NH₂, ═NH, N₃ or NO₂ groups. This expression refers furthermore to groups that may be substituted by one, two, three or more unsubstituted C₁-C₁₀ alkyl, C₂-C₁₀ alkenyl, C₂-C₁₀ alkynyl, C₁-C₁₀ heteroalkyl, C₃-C₁₆ cycloalkyl, C₂-C₁₇ heterocycloalkyl, C₄-C₂₀ alkylcycloalkyl, C₂-C₁₉ heteroalkylcycloalkyl, C₆-C₁₈ aryl, C₁₋₁₇ heteroaryl, C₇-C₂₀ aralkyl or C₂-C₁₉ heteroaralkyl groups. This expression refers furthermore especially to groups that may be substituted by one, two, three or more unsubstituted C₁-C₆ alkyl, C₂-C₆ alkenyl, C₂-C₆ alkynyl, C₁-C₆ heteroalkyl, C₃-C₁₀ cycloalkyl, C₂-C₉ heterocycloalkyl, C₇-C₁₂ alkylcycloalkyl, C₂-C₁₁ heteroalkylcycloalkyl, C₆-C₁₀ aryl, C₁-C₉ heteroaryl, C₇-C₁₂ aralkyl or C₂-C₁₁ heteroaralkyl groups.

Exemplary substituents are F, Cl, Br, OH, SH, ═O, NH₂, C₁₋₄ alkyl, C₁₋₄ heteroalkyl cyclopropyl, SF₅, NO and NO₂.

Other exemplary substituents are F, Cl, Br, OH, SH, ═O, NH₂, C₁₋₄ alkyl (e.g. methyl, ethyl, t-butyl), NMe₂, CONH₂, CH₂NMe₂, NHSO₂Me, C(CH₃)₂CN, COMe, OMe, SMe, COOMe, COOEt, CH₂COOH, OCH₂COOH, COOH, SOMe, SO₂Me, cyclopropyl, SO₂NH₂, SO₂NHMe, SO₂CH₂CH₂OH, NHCH₂CH₂OH, CH₂CH₂OCH₃, SF₅, SO₂NMe₂, NO, NO₂, OCF₃, SO₂CF₃, CN or CF₃.

Other exemplary substituents are F, Cl, Br, Me, OMe, CN or CF₃.

The term halogen preferably refers to F, Cl, Br or I.

According to certain embodiments, all alkyl, alkenyl, alkynyl, heteroalkyl, aryl, heteroaryl, cycloalkyl, heterocycloalkyl, alkylcycloalkyl, heteroalkylcycloalkyl, aralkyl and heteroaralkyl groups described herein may optionally be substituted.

When an aryl, heteroaryl, cycloalkyl, alkylcycloalkyl, heteroalkylcycloalkyl, heterocycloalkyl, aralkyl or heteroaralkyl group contains more than one ring, these rings may be bonded to each other via a single or double bond or these rings may be annulated.

### Compounds

In an aspect, the invention provides compounds of formula (I).

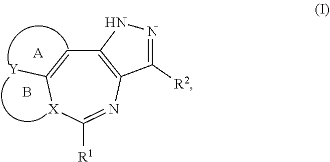

or a tautomer thereof, or a pharmaceutically acceptable salt thereof,
wherein:
R¹ is aryl or heteroaryl;
R² is H, halo, OH, CN, CF₃, C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, or heteroaryl;
A is aryl, heteroaryl, cycloalkyl, heterocycloalkyl, cycloalkenyl, or heterocycloalkenyl;
B is aryl, heteroaryl, cycloalkyl, heterocycloalkyl, cycloalkenyl, or heterocycloalkenyl;
X is C or N; and
Y is C or N,
   wherein each of the substituents in each of R¹, R², A, and B is independently optionally substituted with one or more moieties selected from the group consisting of halo, OH, CN, CF₃, NH₂, NO₂, C₁₋₆ alkyl, C₁₋₆ haloalkyl, C₁₋₆ thioalkyl, C₃₋₈ cycloalkyl, C₂₋₈ heterocycloalkyl, C₂₋₈ heterocycloalkenyl, C₂₋₆ alkenyl, C₂₋₆ alkynyl, C₁₋₆ alkoxy, C₁₋₆ haloalkoxy, C₁₋₆ alkylamino, C₂₋₆ dialkylamino, C₇₋₁₂ aralkyl, C₁₋₁₂ heteroaralkyl, aryl, heteroaryl, —C(O)R, —C(O)OR, —C(O)NRR′, —C(O)NRS(O)₂R′, —C(O)NRS(O)₂NR′R″, —OR, —OC(O)NRR′, —NRR′, —NRC(O)R′, —NRC(O)NR′R″, —NRS(O)₂R′, —NRS(O)₂NR′R″, —S(O)₂R, and —S(O)₂NRR′,
in which each of R, R′, and R″, independently, is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl, or R and R′, or R′ and R″, together with the nitrogen to which they are attached, form C₂₋₈ heterocycloalkyl.

Each of A and B may independently have a defined number of atoms. For example, each of A and B may independently have 4-7 atoms, 5-7 atoms, 6-7 atoms, 4-6 atoms, 5-6 atoms, or 4-5 atoms.

R¹ may be an optionally substituted 6-membered aryl or heteroaryl, or may be an optionally substituted 10-member bicyclic aryl or heteroaryl. The heteroaryl may be a pyridine. R¹ may be substituted with 1-3 of a -Me, ester (for example —OMe), halogen, alcohol (for example —OH or —COH), cyano, —CF₃, CHF₂, —OCHF₂, or cyclopropyl group. R₁ may have the structure:

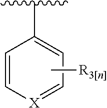

wherein X is carbon or nitrogen, and X may be in an ortho, meta, or para position on the ring,
where n is 0, 1, 2, or 3, and
wherein each R₃ may be independently selected from a -Me, ester (for example —OMe), halogen, alcohol (for example —OH or —COH), cyano, —CF₃, CHF₂, —OCHF₂, or cyclopropyl group.

The compound of formula (I) may have a structure selected from the group consisting of Compounds 301-319 or 340-408 and tautomers thereof:

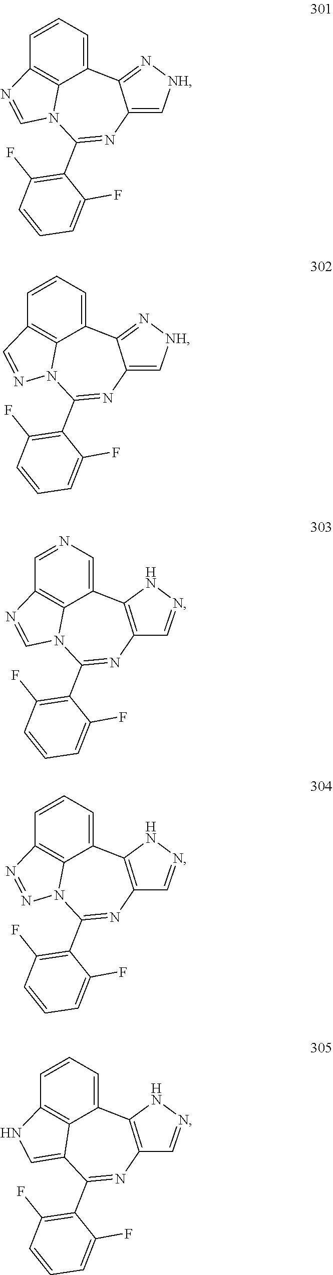

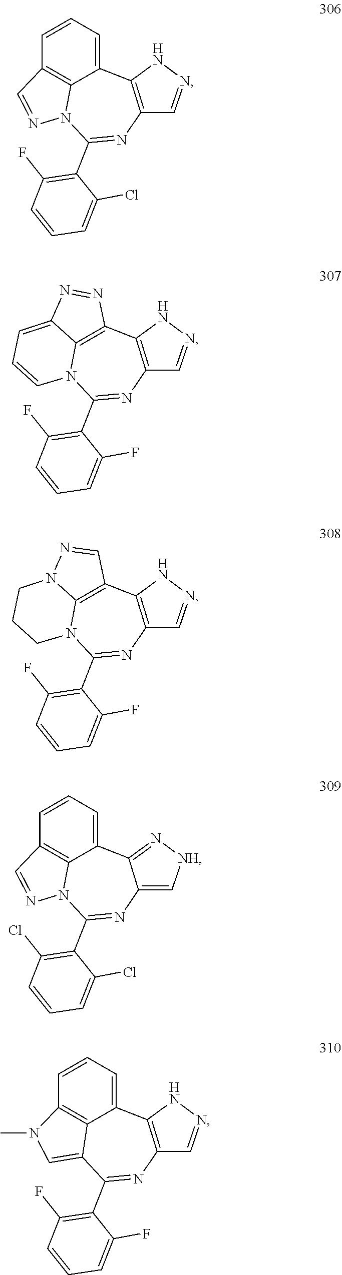

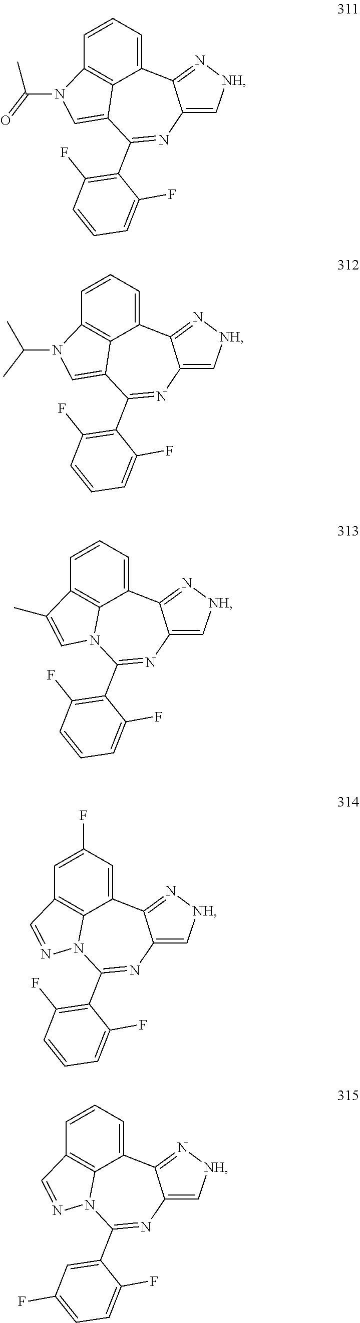

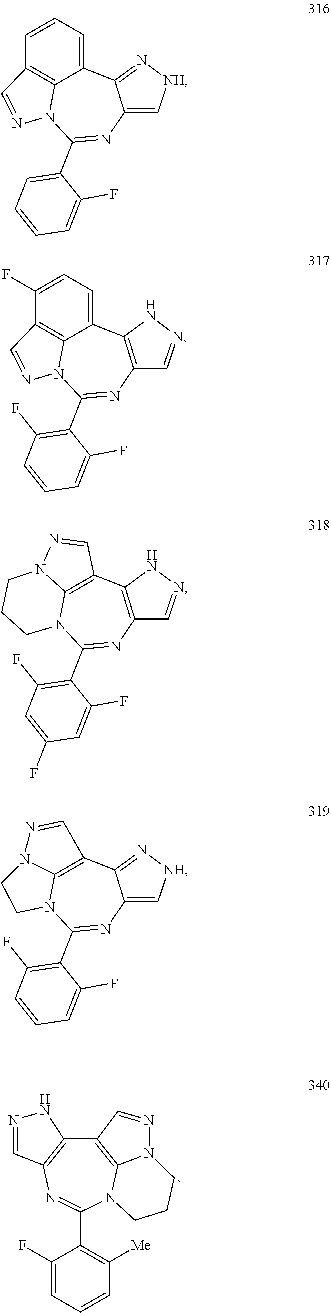

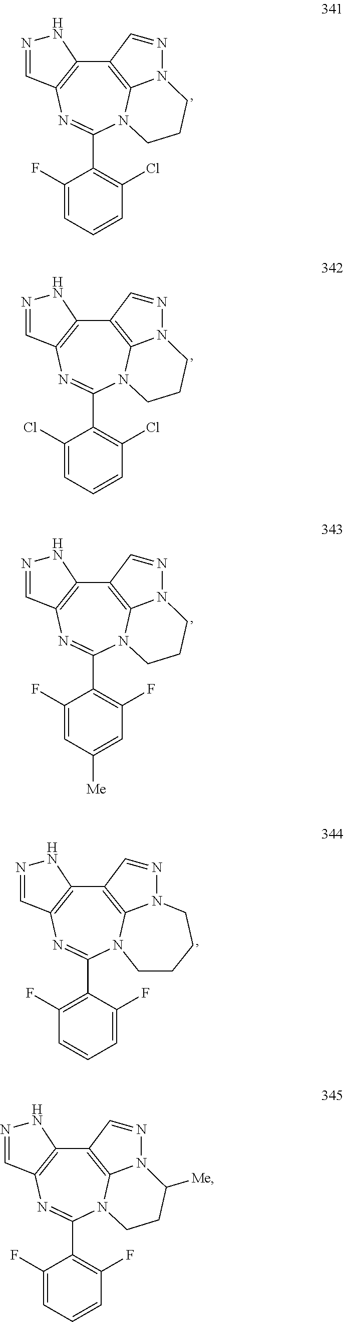 [SMILES: Fc1cccc(Cl)c1C1=Nc2cn[nH]c2-c2cnn3c2N1CCC3 ; Clc1cccc(Cl)c1C1=Nc2cn[nH]c2-c2cnn3c2N1CCC3 ; Cc1cc(F)c(C2=Nc3cn[nH]c3-c3cnn4c3N2CCC4)c(F)c1 ; Fc1cccc(F)c1C1=Nc2cn[nH]c2-c2cnn3c2N1CCCC3 ; CC1CCN2C(c3c(F)cccc3F)=Nc3cn[nH]c3-c3cnn1c32]

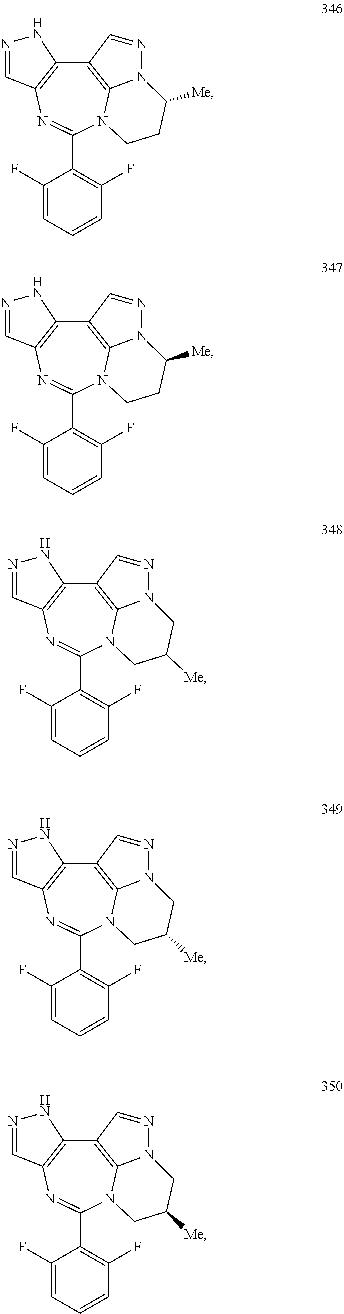 [SMILES: C[C@@H]1CCN2C(c3c(F)cccc3F)=Nc3cn[nH]c3-c3cnn1c32 ; C[C@H]1CCN2C(c3c(F)cccc3F)=Nc3cn[nH]c3-c3cnn1c32 ; CC1CN2C(c3c(F)cccc3F)=Nc3cn[nH]c3-c3cnn(c32)C1 ; C[C@@H]1CN2C(c3c(F)cccc3F)=Nc3cn[nH]c3-c3cnn(c32)C1 ; C[C@H]1CN2C(c3c(F)cccc3F)=Nc3cn[nH]c3-c3cnn(c32)C1]

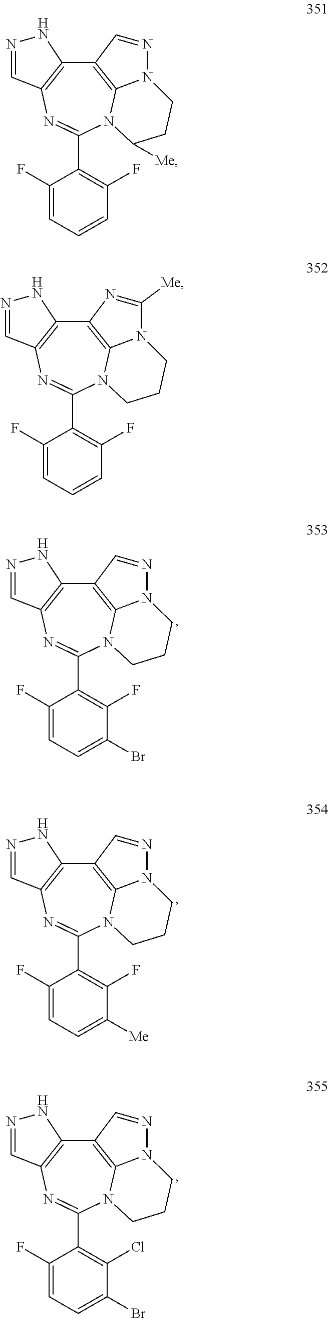 [SMILES: CC1CCn2ncc3c2N1C(c1c(F)cccc1F)=Nc1cn[nH]c1-3 ; Cc1nc2c3n1CCCN3C(c1c(F)cccc1F)=Nc1cn[nH]c1-2 ; Fc1ccc(Br)c(F)c1C1=Nc2cn[nH]c2-c2cnn3c2N1CCC3 ; Cc1ccc(F)c(C2=Nc3cn[nH]c3-c3cnn4c3N2CCC4)c1F ; Fc1ccc(Br)c(Cl)c1C1=Nc2cn[nH]c2-c2cnn3c2N1CCC3]

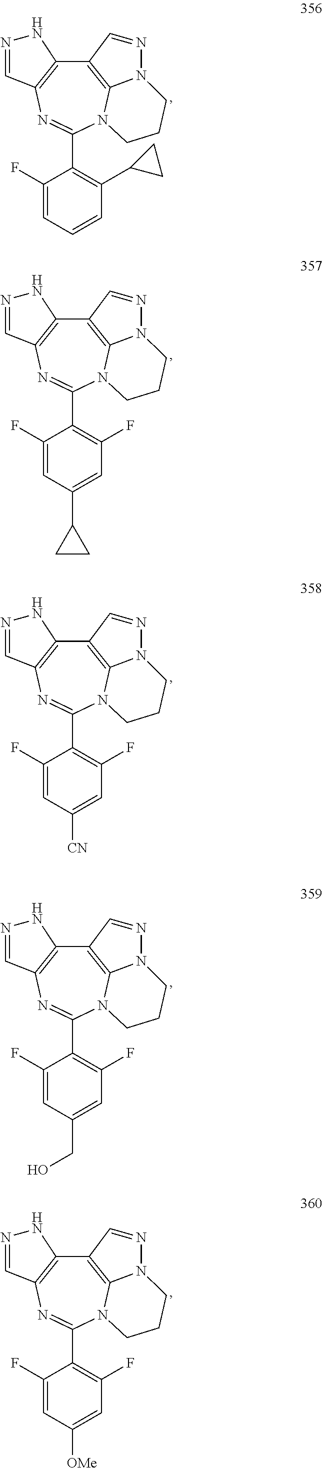 [SMILES: Fc1cccc(C2CC2)c1C1=Nc2cn[nH]c2-c2cnn3c2N1CCC3 ; Fc1cc(C2CC2)cc(F)c1C1=Nc2cn[nH]c2-c2cnn3c2N1CCC3 ; N#Cc1cc(F)c(C2=Nc3cn[nH]c3-c3cnn4c3N2CCC4)c(F)c1 ; OCc1cc(F)c(C2=Nc3cn[nH]c3-c3cnn4c3N2CCC4)c(F)c1 ; COc1cc(F)c(C2=Nc3cn[nH]c3-c3cnn4c3N2CCC4)c(F)c1]

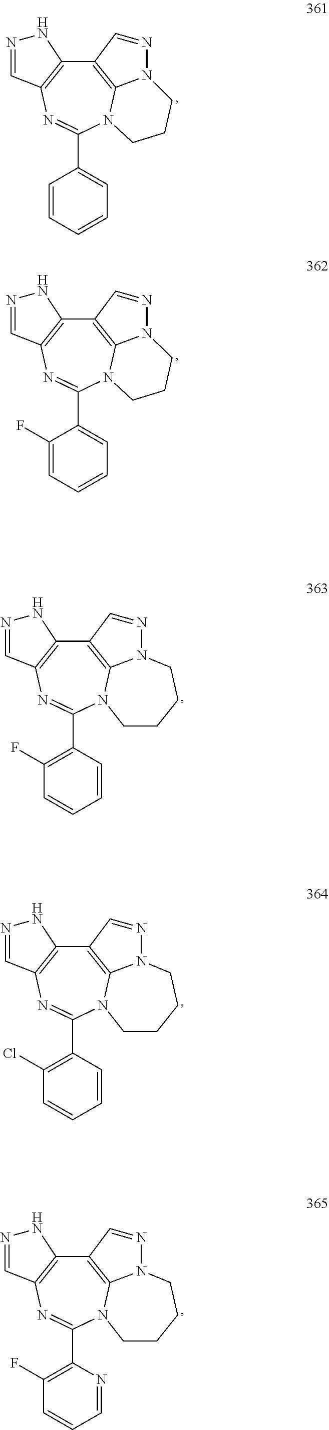 [SMILES: c1ccc(C2=Nc3cn[nH]c3-c3cnn4c3N2CCC4)cc1 ; Fc1ccccc1C1=Nc2cn[nH]c2-c2cnn3c2N1CCC3 ; Fc1ccccc1C1=Nc2cn[nH]c2-c2cnn3c2N1CCCC3 ; Clc1ccccc1C1=Nc2cn[nH]c2-c2cnn3c2N1CCCC3 ; Fc1cccnc1C1=Nc2cn[nH]c2-c2cnn3c2N1CCCC3]

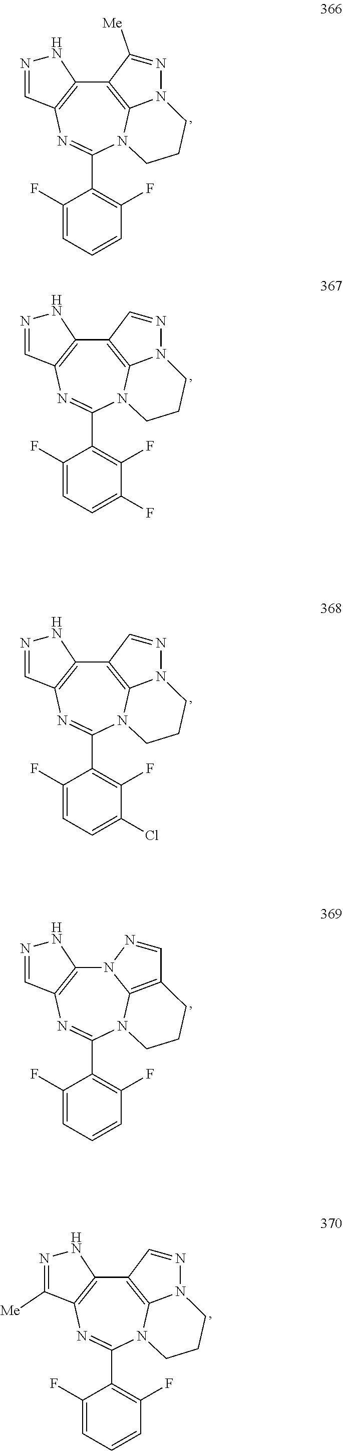 [SMILES: Cc1nn2c3c1-c1[nH]ncc1N=C(c1c(F)cccc1F)N3CCC2 ; Fc1ccc(F)c(C2=Nc3cn[nH]c3-c3cnn4c3N2CCC4)c1F ; Fc1ccc(Cl)c(F)c1C1=Nc2cn[nH]c2-c2cnn3c2N1CCC3 ; Fc1cccc(F)c1C1=Nc2cn[nH]c2-n2ncc3c2N1CCC3 ; Cc1n[nH]c2c1N=C(c1c(F)cccc1F)N1CCCn3ncc-2c31]

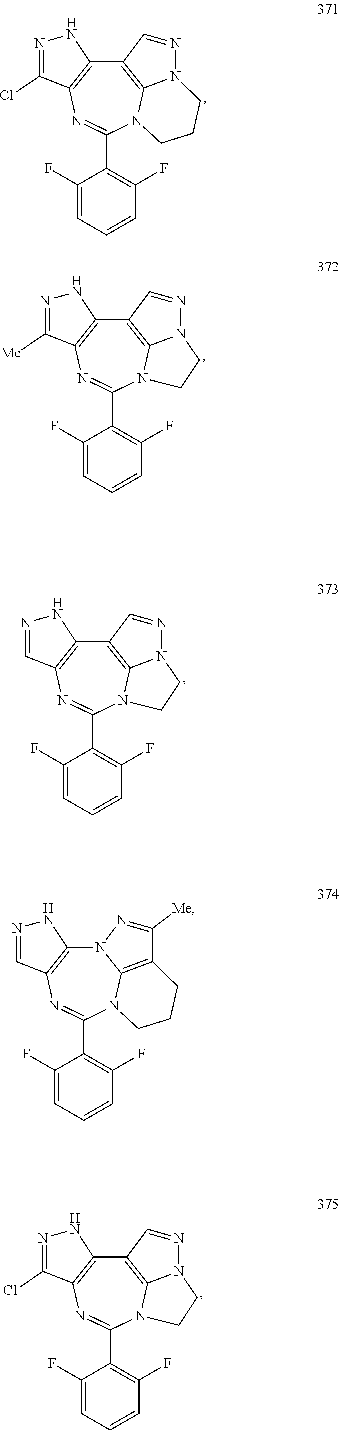 [SMILES: Fc1cccc(F)c1C1=Nc2c(Cl)n[nH]c2-c2cnn3c2N1CCC3 ; Cc1n[nH]c2c1N=C(c1c(F)cccc1F)N1CCn3ncc-2c31 ; Fc1cccc(F)c1C1=Nc2cn[nH]c2-c2cnn3c2N1CC3 ; Cc1nn2c3c1CCCN3C(c1c(F)cccc1F)=Nc1cn[nH]c1-2 ; Fc1cccc(F)c1C1=Nc2c(Cl)n[nH]c2-c2cnn3c2N1CC3]

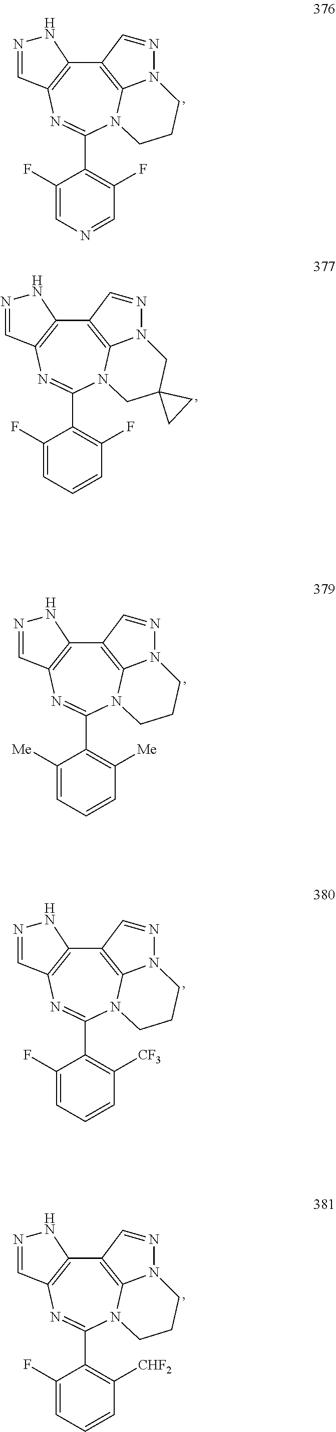 [SMILES: Fc1cncc(F)c1C1=Nc2cn[nH]c2-c2cnn3c2N1CCC3 ; Fc1cccc(F)c1C1=Nc2cn[nH]c2-c2cnn3c2N1CC1(CC1)C3 ; Cc1cccc(C)c1C1=Nc2cn[nH]c2-c2cnn3c2N1CCC3 ; Fc1cccc(C(F)(F)F)c1C1=Nc2cn[nH]c2-c2cnn3c2N1CCC3 ; Fc1cccc(C(F)F)c1C1=Nc2cn[nH]c2-c2cnn3c2N1CCC3]

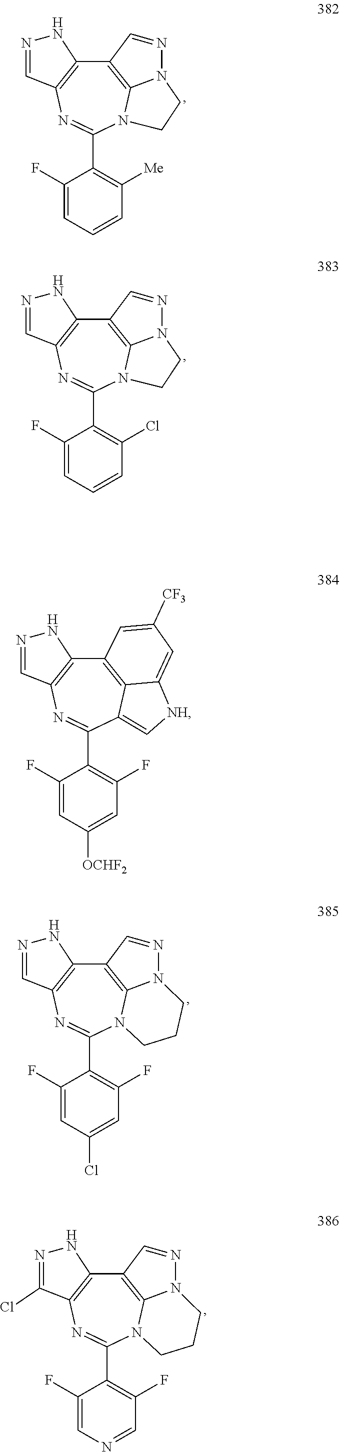 [SMILES: Cc1cccc(F)c1C1=Nc2cn[nH]c2-c2cnn3c2N1CC3 ; Fc1cccc(Cl)c1C1=Nc2cn[nH]c2-c2cnn3c2N1CC3 ; Fc1cc(OC(F)F)cc(F)c1C1=Nc2cn[nH]c2-c2cc(C(F)(F)F)cc3[nH]cc1c23 ; Fc1cc(Cl)cc(F)c1C1=Nc2cn[nH]c2-c2cnn3c2N1CCC3 ; Fc1cncc(F)c1C1=Nc2c(Cl)n[nH]c2-c2cnn3c2N1CCC3]

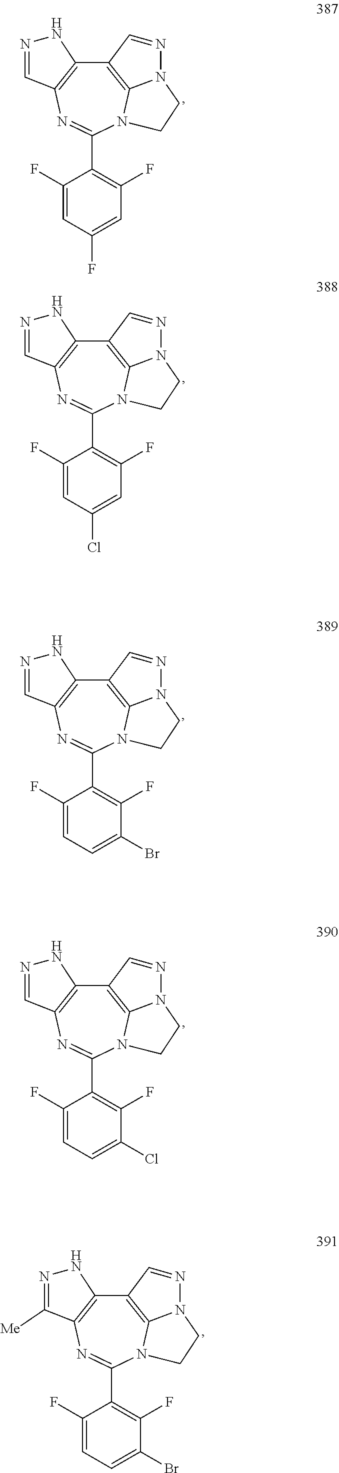 [SMILES: Fc1cc(F)c(C2=Nc3cn[nH]c3-c3cnn4c3N2CC4)c(F)c1 ; Fc1cc(Cl)cc(F)c1C1=Nc2cn[nH]c2-c2cnn3c2N1CC3 ; Fc1ccc(Br)c(F)c1C1=Nc2cn[nH]c2-c2cnn3c2N1CC3 ; Fc1ccc(Cl)c(F)c1C1=Nc2cn[nH]c2-c2cnn3c2N1CC3 ; Cc1n[nH]c2c1N=C(c1c(F)ccc(Br)c1F)N1CCn3ncc-2c31]

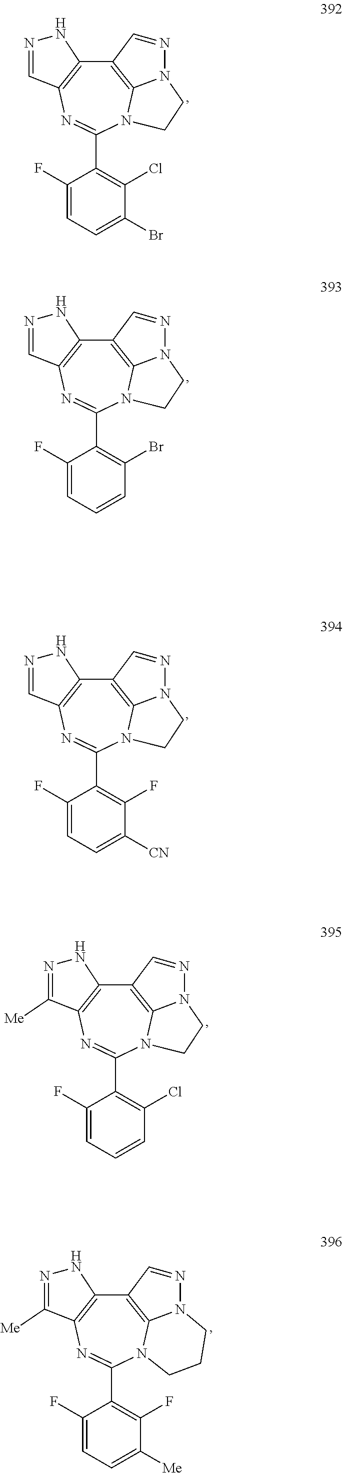 [SMILES: Fc1ccc(Br)c(Cl)c1C1=Nc2cn[nH]c2-c2cnn3c2N1CC3 ; Fc1cccc(Br)c1C1=Nc2cn[nH]c2-c2cnn3c2N1CC3 ; N#Cc1ccc(F)c(C2=Nc3cn[nH]c3-c3cnn4c3N2CC4)c1F ; Cc1n[nH]c2c1N=C(c1c(F)cccc1Cl)N1CCn3ncc-2c31 ; Cc1ccc(F)c(C2=Nc3c(C)n[nH]c3-c3cnn4c3N2CCC4)c1F]

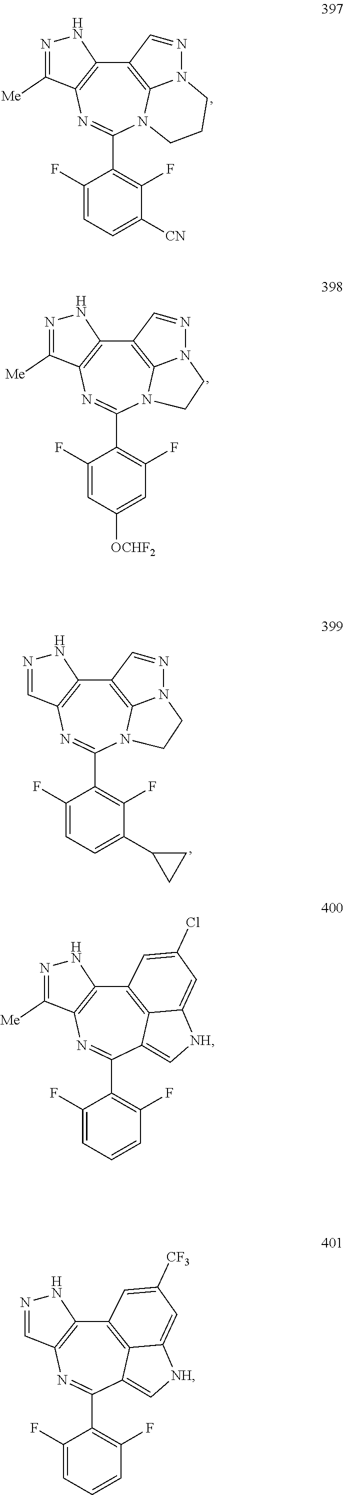

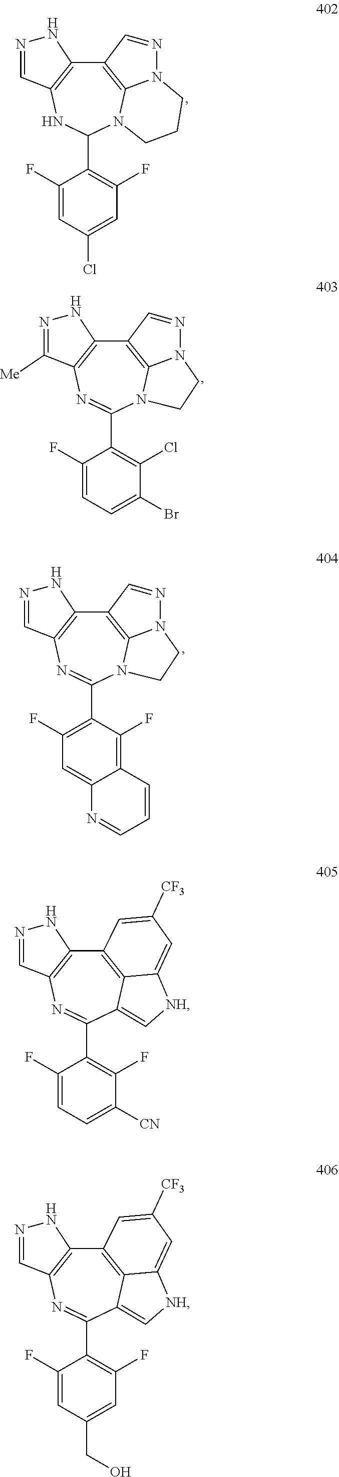

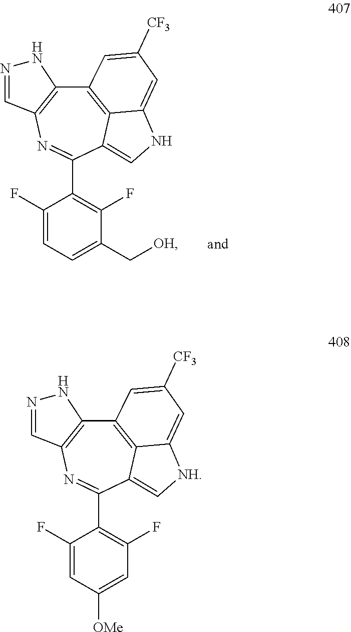 [SMILES: OCc1ccc(F)c(C2=Nc3cn[nH]c3-c3cc(C(F)(F)F)cc4[nH]cc2c34)c1F ; "and" ; COc1cc(F)c(C2=Nc3cn[nH]c3-c3cc(C(F)(F)F)cc4[nH]cc2c34)c(F)c1]

### Pharmaceutical Compositions

The present invention provides pharmaceutical compositions comprising one or more compounds of described above or a pharmaceutically acceptable ester, prodrug, hydrate, solvate or salt thereof, optionally in combination with a pharmaceutically acceptable carrier. The invention further provides such compounds for the preparation of a medicament for the treatment of one or more diseases mentioned herein.

A pharmaceutical composition may contain one or more compounds of the invention in a therapeutically effective amount. A therapeutically effective amount of a compound in accordance with this invention means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage may be adjusted to the individual requirements in each particular case including the specific compound being administered, the route of administration, the condition being treated, as well as the patient being treated.

Compositions of the invention may include a vehicle for delivery of one or more compounds of the invention. For example, the composition may contain particles, such as nanoparticles, microparticles, liposomes, micelles, and virus particles.

Examples of pharmacologically acceptable salts of sufficiently basic compounds of the invention are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acidic compound of the invention may form alkali or earth alkali metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, meglumin, piperidine, morpholine, tris-(2-hydroxyethyl)amine, lysine or arginine salts; all of which are also further examples of salts of the invention. Compounds of the invention may be solvated, especially hydrated. The hydratization/hydration may occur during the process of production or as a consequence of the hygroscopic nature of the initially water free compounds of the invention. The solvates and/or hydrates may e.g. be present in solid or liquid form.

It should be appreciated that certain compounds of the invention may have tautomeric forms from which only one might be specifically mentioned or depicted in the following description, different geometrical isomers (which are usually denoted as cis/trans isomers or more generally as (E) and (Z) isomers) or different optical isomers as a result of one or more chiral carbon atoms (which are usually nomenclatured under the Cahn-Ingold-Prelog or R/S system). All these tautomeric forms, geometrical or optical isomers (as well as racemates and diastereomers) and polymorphous forms are included in the invention. Since the compounds of the invention may contain asymmetric C-atoms, they may be present either as achiral compounds, mixtures of diastereomers, mixtures of enantiomers or as optically pure compounds. The present invention comprises both all pure enantiomers and all pure diastereomers, and also the mixtures thereof in any mixing ratio.

According to a further embodiment of the present invention, one or more hydrogen atoms of the compounds of the present invention may be replaced by deuterium or tritium. Deuterium or tritium modification may improve the metabolic properties of a drug with little or no change in its intrinsic pharmacology. Deuterium or tritium substitution at specific molecular positions may improve metabolic stability, reduce formation of toxic metabolites and/or increases the formation of desired active metabolites. Accordingly, the present invention also encompasses the partially and fully deuterated or tritiated compounds of the invention. The term hydrogen also encompasses deuterium and tritium.

The therapeutic use of compounds according to the invention, their pharmacologically acceptable salts, solvates and hydrates, respectively, as well as formulations and pharmaceutical compositions also lie within the scope of the present invention. The pharmaceutical compositions according to the present invention may comprise at least one compound of the invention as an active ingredient and, optionally, carrier substances and/or adjuvants.

The present invention also relates to pro-drugs which are composed of a compound of the invention and at least one pharmacologically acceptable protective group which will be cleaved off under physiological conditions, such as an alkoxy-, arylalkyloxy-, acyl-, acyloxymethyl group (e.g. pivaloyloxymethyl), an 2-alkyl-, 2-aryl- or 2-arylalkyl oxycarbonyl-2-alkylidene ethyl group or an acyloxy group as defined herein, e.g. ethoxy, benzyloxy, acetyl or acetyloxy or, especially for a compound of the invention, carrying a hydroxy group (—OH): a sulfate, a phosphate (—OPO₃ or —OCH₂OPO₃) or an ester of an amino acid. For example, compositions may contain pro-drugs of the hydroxy group of a compound of the invention.

As used herein, the term pharmaceutically acceptable ester especially refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The present invention also relates to a prodrug, a biohydrolyzable ester, a biohydrolyzable amide, a polymorph, tautomer, stereoisomer, metabolite, N-oxide, biohydrolyzable carbamate, biohydrolyzable ether, physiologically functional derivative, atropisomer, or in vivo-hydrolysable precursor, diastereomer or mixture of diastereomers, chemically protected form, affinity reagent, complex, chelate and a stereoisomer of the compounds of the invention.

As mentioned above, therapeutically useful agents that contain compounds of the invention, their solvates, salts or formulations are also comprised in the scope of the present invention. In general, compounds of the invention will be administered by using the known and acceptable modes known in the art, either alone or in combination with any other therapeutic agent.

For oral administration such therapeutically useful agents can be administered by one of the following routes: oral, e.g. as tablets, dragees, coated tablets, pills, semisolids, soft or hard capsules, for example soft and hard gelatine capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containing the active ingredient or intranasal. For the production of such tablets, pills, semisolids, coated tablets, dragees and hard, e.g. gelatine, capsules, the therapeutically useful product may be mixed with pharmaceutically inert, inorganic or organic excipients as are e.g. lactose, sucrose, glucose, gelatine, malt, silica gel, starch or derivatives thereof, talc, stearinic acid or their salts, dried skim milk, and the like. For the production of soft capsules one may use excipients as are e.g. vegetable, petroleum, animal or synthetic oils, wax, fat, polyols. For the production of liquid solutions, emulsions or suspensions or syrups one may use as excipients e.g. water, alcohols, aqueous saline, aqueous dextrose, polyols, glycerin, lipids, phospholipids, cyclodextrins, vegetable, petroleum, animal or synthetic oils. Particularly useful are lipids, such as phospholipids (e.g., natural origin and/or with a particle size between 300 to 350 nm) in phosphate buffered saline (pH=7 to 8, e.g., 7.4). For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilization, e.g. UV stabilizers, emulsifiers, sweetener, aromatizers, salts to change the osmotic pressure, buffers, coating additives and antioxidants.

In general, in the case of oral or parenteral administration to adult humans weighing approximately 80 kg, a daily dosage of about 10 mg to about 10,000 mg, or from about 20 mg to about 1,000 mg, should be appropriate, although the upper limit may be exceeded when indicated. The daily dosage can be administered as a single dose or in divided doses, or for parenteral administration, it may be given as continuous infusion or subcutaneous injection.

### Methods of Making Compounds

The invention also provides methods of making compounds of the invention, such as those described above. Synthesis schemes for making specific compounds of formula (I) are provided in the Examples below.

### Methods of Treating Diseases, Disorders, and Conditions

The compounds and compositions of the invention may be used to diagnose, treat, or prevent a disease, disorder, or condition. The invention further provides methods of using the compounds or compositions of the invention to diagnose or treat a disease, disorder, or condition.

Diseases, disorders, and conditions that can be diagnosed and/or treated using compositions and methods of the invention include those associated with aberrant activity, e.g., increased activity or decreased activity, of one or more kinases. The kinase may be a serine-threonine kinase or a tyrosine kinase, e.g., a receptor tyrosine kinase or non-receptor tyrosine kinase. For example, and without limitation, the kinase may be leucine-rich repeat kinase 2 (LRRK2), NUAK family SNF1-like kinase 1 (NUAK1, also known as AMPK-related protein kinase 5 or ARK5), or non-receptor tyrosine-protein kinase TYK2 (TYK2), including mutants of any of the aforementioned kinases.

The disease, disorder, or condition may be associated with aberrant LRRK2 activity, such as Alzheimer's disease, Crohn's disease, inflammatory bowel disease, an inflammatory disease, leprosy, neurodegenerative diseases, a non-skin cancer, or Parkinson's disease, including familial Parkinson's disease, sporadic Parkinson's disease, late-onset Parkinson's disease (PD), and type 8 Parkinson's disease.

The disease, disorder, or condition may be associated with aberrant NUAK1 activity, such as cancer, e.g., colorectal cancer, stomach cancer, endometrial cancer, or multiple myeloma, diabetes, fibrosis, a neurodegenerative disease, or omphalocele.

The disease, disorder, or condition may be associated with aberrant TYK2 activity, such as autoimmune disorders, Crohn's disease, hyperimmunoglobulin E syndrome, inflammatory bowel disease, multiple sclerosis (MS), multiple sclerosis (MS), psoriasis, rheumatoid arthritis, systemic lupus erythematosus (SLE), type 1 diabetes (TID), or ulcerative colitis.

The disease, disorder, or condition may be or include a respiratory tract/obstructive airways disease or disorder, such as rhinorrhea, tracheal constriction, airway contraction, acute-, allergic, atrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis), pollinosis, asthma (such as bronchial, atopic, allergic, intrinsic, extrinsic, exercise-induced, cold air-induced, occupational, bacterial infection-induced, and dust asthma particularly chronic or inveterate asthma (e.g. late asthma and airways hyper-responsiveness)), bronchitis (including chronic, acute, arachidic, catarrhal, croupus, phthinoid and eosinophilic bronchitis), cardiobronchitis, pneumoconiosis, chronic inflammatory disease of the lung which result in interstitial fibrosis, such as interstitial lung disease (ILD) (e.g., idiopathic pulmonary fibrosis, or ILD associated with rheumatoid arthritis, or other autoimmune conditions), acute lung injury (ALI), adult respiratory distress syndrome (ARDS), chronic obstructive pulmonary, airways or lung disease (CORD, COAD, COLD or COPD, such as irreversible COPD), chronic sinusitis, conjunctivitis (e.g. allergic conjunctivitis), cystic fibrosis, extrinsic allergic alveolitis (like farmer's lung and related diseases), fibroid lung, hypersensitivity lung diseases, hypersensitivity pneumonitis, idiopathic interstitial pneumonia, nasal congestion, nasal polyposis, otitis media, and cough (chronic cough associated with inflammation or iatrogenic induced), pleurisy, pulmonary congestion, emphysema, bronchiectasis, sarcoidosis, lung fibrosis, including cryptogenic fibrosing alveolitis, fibrosis complicating anti-neoplastic therapy and chronic infection, including tuberculosis and aspergillosis and other fungal infections, vasculitic and thrombotic disorders of the lung vasculature, and pulmonary hypertension, acute viral infection including the common cold, and infection due to respiratory syncytial virus, influenza, coronavirus (including SARS) and adenovirus, allergic bronchopulmonary mycosis, emphysema, diffuse panbronchiolitis, systemic anaphylaxis or hypersensitivity responses, drug allergies (e.g., to penicillin, cephalosporins), insect sting allergies, and food related allergies which may have effects remote from the gut (such as migraine, rhinitis and eczema), anaphylactic shock, or vascular spasms.

The disease, disorder, or condition may be or include a bone and joint related disease or disorder, such as osteoporosis, arthritis (including rheumatic, infectious, autoimmune, chronic, malignant), seronegative spondyloarthropathies (such as ankylosing spondylitis, rheumatoid spondylitis, psoriatic arthritis, enthesopathy, Bechet's disease, Marie-Strumpell arthritis, arthritis of inflammatory bowel disease, and Reiter's disease), systemic sclerosis, osteoarthritis, osteoarthrosis, both primary and secondary to e.g. congenital hip dysplasia, cervical and lumbar spondylitis, and low back and neck pain, Still's disease, reactive arthritis and undifferentiated spondarthropathy, septic arthritis and other infection-related arthropathies and bone disorders such as tuberculosis, including Pott's disease and Poncet's syndrome, acute and chronic crystal-induced synovitis including urate gout, calcium pyrophosphate deposition disease, and calcium apatite related tendon, bursar and synovial inflammation, primary and secondary Sjogren's syndrome, systemic sclerosis and limited scleroderma, mixed connective tissue disease, and undifferentiated connective tissue disease, inflammatory myopathies including, polymalgia rheumatica, juvenile arthritis including idiopathic inflammatory arthritides of whatever joint distribution and associated syndromes, other joint disease (such as intervertebral disc degeneration or temporomandibular joint degeneration), rheumatic fever and its systemic complications, vasculitides including giant cell arteritis, Takayasu's arteritis, polyarteritis nodosa, microscopic polyarteritis, and vasculitides to associated with viral infection, hypersensitivity reactions, cryoglobulins, paraproteins, low back pain, Familial Mediterranean fever, Muckle-Wells syndrome, and Familial Hibenian Fever, Kikuchi disease, drug-induced arthalgias, tendonititides, polychondritis, and myopathies, osteoporosis, osteomalacia like osteoporosis, osteopenia, osteogenesis imperfects, osteopetrosis, osteofibrosis, osteonecrosis, Paget's disease of bone, hypophosphatemia, Felty's syndrome, Still's disease, slack of artificial joint implant, sprain or strain of muscle or joint, tendinitis, fasciitis, periarthritis humeroscapularis, cervico-omo-brachial syndrome, or tenosynovitis.

The disease, disorder, or condition may be or include a skin or eye related disease or disorder, such as glaucoma, ocular hypertension, cataract, retinal detachment, psoriasis (including psoriasis vulgaris, pustular psoriasis, arthritic psoriasis, erythroderma psoriaticum), palmoplantar pustulosis, xerodoma, eczematous diseases (like atopic dermatitis, ultraviolet radiation dermatitis, contact dermatitis, and seborrheic dermatitis), phytodermatitis, photodermatitis, cutaneous eosinophilias, chronic skin ulcers, cutaneous lupus erythematosus, contact hypersensitivity/allergic contact dermatitis (including sensitivity to poison ivy, sumac, or oak), and eosinophilic folliculitis (Ofuji's disease), pruritus, drug eruptions, urticaria (acute or chronic, allergic or non-allergic), acne, erythema, dermatitis herpetiformis, scleroderma, vitiligo, lichen planus, lichen sclerosus et atrophica, pyodenna gangrenosum, skin sarcoid, pemphigus, ocular pemphigus, pemphigoid, epidermolysis bullosa, angioedema, vasculitides, toxic erythemas, cutaneous eosinophilias, alopecia areata, male-pattern baldness, Sweet's syndrome, Stevens-Johnson syndrome, Weber-Christian syndrome, erythema multiforme, cellulitis, both, infective and non infective, panniculitis, cutaneous Lymphomas, nonr melanoma skin cancer and other dysplastic lesions, blepharitis, iritis, anterior and posterior uveitis, choroiditis, autoimmune, degenerative or inflammatory disorders affecting the retina, ophthalmitis including sympathetic ophthalmitis, sarcoidosis, xerosis infections including viral, fungal, and bacterial, allergic conjunctivitis, increased fibrosis, keloids, keloplasty, post surgical scars, epidermolysis bullosa, dry eye, ocular inflammation, allergic conjunctivitis, vernal conjunctivitis, vernal keratoconjunctivitis, and giant papillary conjunctivitis, ocular angiogenesis, cornea damage and scar, all forms of macular degeneration, macular edema, macular dystrophy, abnormal wound healing, scleritis, episcleritis, pachydermia, peripheral ulcerative keratitis, fungal keratitis, herpetic keratitis, invasive aspergillosis; conical cornea, dystorphia epithelialis comeae, or severe intraocular inflammation.

The disease, disorder, or condition may be or include a gastrointestinal tract and abdominal related disease or disorder, such as celiac/coeliac disease (e.g. celiac sprue), cholecystitis, enteritis (including infectious, ischemic, radiation, drug-induced, and eosinophilic gastroenteritis), eosinophilic esophagitis, eosinophilic gastrointestinal inflammation, allergen induced diarrhea, enteropathy associated with seronegative arthropathies, gastritis, autoimmune atrophic gastritis, ischemic bowel disease, inflammatory bowel disease (Crohn's disease and ulcerative colitis), colitis, Mooren's ulcer, irritable bowel syndrome, necrotizing enterocolitis, gut ischemia, glossitis, gingivitis, periodontitis, oesophagitis, including reflex, proctitis, fibrosis and cirrhosis of the liver, pancreatitis, both acute and chronic, pancreatic fibrosis, pancreatic sclerosis, pancreatolithiasis, hepatic cirrhosis, hepatitis (congestive, autoimmune, acute, fulminant, chronic, drug-induced, alcoholic, lupoid, steatohepatitis and chronic viral), fatty liver, primary biliary cirrhosis, hepatic porphyria, and gastrointestinal related allergic disorders, spastic colon, diverticulitis, gastroenteric bleeding, Behcet's disease; partial liver resection, acute liver necrosis (e.g. necrosis caused by toxins, viral hepatitis, shock or anoxia), or hemolytic uremic syndrome.

The disease, disorder, or condition may be or include a hematological disease or disorder, such as anemias, coagulation, myeloproliferative disorders, hemorrhagic disorders, leukopenia, eosinophilic disorders, leukemias (e.g. myelogenous, lymphomas, plasma cell dyscrasias, disorders of the spleen, Band's disease, hemophilia, purpura (including idiopathic thrombocytopenic purpura), or Wiskott-Aldrich syndrome.

The disease, disorder, or condition may be or include a metabolic disease or disorder, such as obesity, amyloidosis, disturbances of the amino and acid metabolism like branched chain disease, hyperaminoacidemia, hyperaminoaciduria, disturbances of the metabolism of urea, hyperammonemia, mucopolysaccharidoses e.g. Maroteaux-Lamy syndrome, storage disease like glycogen storage diseases and lipid storage diseases, glycogenosis I diseases like Cori's disease, malabsorption diseases like intestinal carbohydrate malabsorption, oligosaccharidase deficiency like maltase-, lactase-, sucrase-insufficiency, disorders of the metabolism of fructose, disorders of the metabolism of galactose, galactosaemia, disturbances of carbohydrate utilization like diabetes, hypoglycemia, disturbances of pyruvate metabolism, hypolipidemia, hypolipoproteinemia, hyperlipidemia, hyperlipoproteinemia, carnitine or carnitine acyltransferase deficiency, disturbances of the porphyrin metabolism, porphyrins, disturbances of the purine metabolism, lysosomal diseases, metabolic diseases of nerves and nervous systems like gangliosidoses, sphingolipidoses, sulfatidoses, leucodystrophies, or Lesch Nyhan syndrome.

The disease, disorder, or condition may be or include a cerebellar dysfunction or disturbance of brain metabolism, such as dementia, Alzheimer's disease, Huntington's chores, Parkinson's disease, Pick's disease, toxic encephalopathy, demyelinating neuropathies like inflammatory neuropathy, Guillain-Barre syndrome; Meniere's disease and radiculopathy, primary and secondary metabolic disorders associated with hormonal defects like any disorder stemming from either an hyperfunction or hypofunction of some hormone-secreting endocrine gland and any combination thereof. Sipple's syndrome, pituitary gland dysfunction and its effects on other endocrine glands, such as the thyroid, adrenals, ovaries, and testes, acromegaly, hyper- and hypothyroidism, euthyroid goiter, euthyroid sick syndrome, thyroiditis, and thyroid cancer, over or underproduction of the adrenal steroid hormones, adrenogenital syndrome, Cushing's syndrome, Addison's disease of the adrenal cortex, Addison's pernicious anemia, primary and secondary aldosteronism, diabetes insipidus, diabetes mellitus, carcinoid syndrome, disturbances caused by the dysfunction of the parathyroid glands, pancreatic islet cell dysfunction, diabetes, disturbances of the endocrine system of the female like estrogen deficiency, resistant ovary syndrome; muscle weakness, myotonia. Duchenne's and other muscular dystrophies, dystrophia myotonica of Steinert, mitochondrial myopathies like disturbances of the catabolic metabolism in the muscle, carbohydrate and lipid storage myopathies, glycogenoses, myoglobinuria, malignant hyperthermia, polymyalgia rheumatics, dermatomyositis, multiple myositis, primary myocardial disease, cardiomyopathy; disorders of the ectoderm, neurofibromatosis, scleroderma and polyar teritis, Louis-Bar syndrome, von Hippel-Lindau disease, Sturge-Weber syndrome, tuberous sclerosis, amyloidosis, porphyria; sexual dysfunction of the male and female; confused states and seizures due to inappropriate secretion of antidiuretic hormone from the pituitary gland, Liddle's syndrome, Bartter's syndrome, Fanconi's I syndrome, or renal electrolyte wasting.

The disease, disorder, or condition may be or include a transplant rejection related condition, such as acute and chronic allograft rejection following solid organ transplant, for example, transplantation of kidney, heart, liver, lung, and cornea, chronic graft versus host disease, skin graft rejection, and bone marrow transplant rejection, or immunosuppression.

The disease, disorder, or condition may be or include a genitourinary related condition, such as nephritis (interstitial, acute interstitial (allergic), and glomerulonephritis), nephrotic syndrome, cystitis including acute and chronic (interstitial) cystitis and Hunner's ulcer, acute and chronic urethritis, prostatitis, epididymitis, oophoritis, salpingitis, vulvo vaginitis, vulvovaginal candidiasis, Peyronie's disease, and erectile dysfunction, renal disease, renal fibrosis, pyelonephritis, secondary contracted kidney, steroid dependent and steroid-resistant nephrosis, or Goodpasture's syndrome.

The disease, disorder, or condition may be or include a CNS related disease or disorder, such as neurodegenerative diseases, Alzheimer's disease and other cementing disorders including CJD and nvCJD, amyloidosis, and other demyelinating syndromes, cerebral atherosclerosis and vasculitis, temporal arteritis, myasthenia gravis, acute and chronic so pain (acute, intermittent or persistent, whether of central or peripheral origin) including post-operative, visceral pain, headache, migraine, neuralgia (including trigeminal), atypical facial pain, joint and bone pain, pain arising from cancer and tumor invasion, neuropathic pain syndromes including diabetic, post-herpetic, and HIV-associated neuropathies, neurosarcoidosis, to brain injuries, cerebrovascular diseases and their consequences, Parkinson's disease, corticobasal degeneration, motor neuron disease, dementia, including ALS (Amyotrophic-lateral sclerosis), multiple sclerosis, traumatic brain injury, stroke, post-stroke, post-traumatic brain injury, and small-vessel cerebrovascular disease, dementias, vascular dementia, dementia with Lewy bodies, frontotemporal dementia and Parkinsonism linked 1 to chromosome 17, frontotemporal dementias, including Pick's disease, progressive supranuclear palsy, corticobasal degeneration, Huntington's disease, thalamic degeneration, HIV dementia, schizophrenia with dementia, and Korsakoffs psychosis, within the meaning of the definition are also considered to be CNS disorders central and peripheral nervous system complications of malignant, infectious or autoimmune processes, algesia, cerebral infarction, attack, cerebral ischemia, head injury, spinal cord injury, myelopathic muscular atrophy, Shy-Drager syndrome, Reye's syndrome, progressive multifocal leukoencephalopathy, normal pressure hydrocephalus, sclerosing panencephalitis, frontal lobe type dementia, acute anterior poliomyelitis (poliomyelitis), poliomyelitis neurosis, viral encephalitis, allergic encephalomyelitis, epileptic encephalopathies, Creutzfeldt-Jakob disease, Kuru disease, bovine spongiform encephalopathy (mad cow disease), scrapie, epilepsy, cerebral amyloid angiopathy, depression, mania, manic-depressive psychosis, hereditary cerebellar ataxia, peripheral neuropathy, Nasu-Hakola syndrome, or Machado-Joseph disease.

The disease, disorder, or condition may be or include an inflammatory or immunological disease or disorder, such as general inflammation (of the ocular, nasal, pulmonary, and gastrointestinal passages), mastocytosis/mast cell disorders (cutaneous, systemic, mast cell activation syndrome, and pediatric mast cell diseases), mastitis (mammary gland), vaginitis, vasculitis (e.g., necrotizing, cutaneous, and hypersensitivity vasculitis), Wegener granulamatosis, myyositis (including polymyositis, dermatomyositis), basophil related diseases including basophilic leukemia and basophilic leukocytosis, and eosinophil related diseases such as Churg-Strauss syndrome, eosinophilic granuloma, lupus erythematosus (such as, systemic lupus erythematosus, subacute cutaneous lupus erythematosus, and discoid lupus erythematosus), chronic thyroiditis, Hashimoto's thyroiditis, Grave's disease, type I diabetes, complications arising from diabetes mellitus, other immune disorders, eosinophilia fasciitis, hyper IgE syndrome, Addison's disease, antiphospholipid syndrome, immunodeficiency disease, acquired immune deficiency syndrome (AIDS), leprosy, Sezary syndrome, paraneoplastic syndromes, and other autoimmune disorders, fervescence, myositis, nervous diseases selected from multiple myositis, bursitis, Evans syndrome, leukotriene B4-mediated diseases, idiopathic hypoparathyroidism, nephrotic syndrome lupus, or immunosuppression.

The disease, disorder, or condition may be or include a cardiovascular disease or disorder, such as congestive heart failure, myocardial infarction, ischemic diseases of the heart, all kinds of atrial and ventricular arrhythmias, hypertension, cerebral trauma, occlusive vascular disease, stroke, cerebrovascular disorder, atherosclerosis, restenosis, affecting the coronary and peripheral is circulation, pericarditis, myocarditis, inflammatory and auto-immune cardiomyopathies including myocardial sarcoid, endocarditis, valvulitis, and aortitis including infective (e.g. syphilitic), hypertensive vascular diseases, peripheral vascular diseases, and atherosclerosis, vasculitides, disorders of the proximal and peripheral veins including phlebitis and thrombosis, including deep vein thrombosis and complications of varicose veins, aortic aneurism, periarteritis nodosa, cardiac fibrosis, post-myocardial infarction, idiopathic cardiomyopathy, or angioplasty.

The disease, disorder, or condition may be or include an oncological disease or disorder, such as common cancers (prostrate, breast, lung, ovarian, pancreatic, bowel and colon, abdomen, stomach (and any other digestive system cancers), liver, pancreas, peritoneum, endocrine glands (adrenal, parathyroid, pituitary, testicles, ovary, thymus, thyroid), eye, head, neck, nervous system (central and peripheral), lymphatic system, blood, pelvic, skin, bone, soft tissue, spleen, thoracic, urogenital, and brain tumors), breast cancer, genitourinary cancer, lung cancer, gastrointestinal cancer, epidermoid cancer, melanoma, ovarian cancer, pancreas cancer, neuroblastoma, malignancies affecting the bone marrow (including the leukaemias) and lymphoproliferative systems, such as Hodgkin's and non-Hodgkin's lymphoma, B-cell lymphoma, follicular lymphoma, metastatic disease and tumour recurrences, and paraneoplastic syndromes, as well as hypergammaglobulinemia, lymphoproliferative diseases, disorders, and/or conditions, paraproteinemias, purpura (including idiopathic thrombocytopenic purpura), Waldenstron's Macroglobulinemia, Gaucher's Disease, histiocytosis, retinoblastoma and any other hyperproliferative disease, sarcomata, cachexia, tumor growth, tumor invasion, metastasis, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma, T-cell lymphoma or cutaneous T-cell lymphoma). Myeloid cancer includes e.g. acute or chronic myeloid leukaemia, or keratoleukoma.

The disease, disorder, or condition may be or include another disease or disorder, such as pain, migraine, sleep disorders, fever, sepsis, idiopathic thrombocytopenia pupura, post-operative adhesions, flushing, ischemic/reperfusion injury in the heart, brain, peripheral limbs, bacterial infection, viral infection, fungal infection, thrombosis, endotoxin shock, septic shock, thermal regulation including fever, Raynaud's disease, gangrene, diseases requiring anti-coagulation therapy, congestive heart failure, mucus secretion disorders, pulmonary hypotension, prostanoid-induced smooth muscle contract associated with dysmenorrhea and premature labor, premature delivery, reperfusion injury, burn, thermal injury, hemorrhage or traumatic shock, menstrual pain, menstrual cramp, dysmenorrhea, periodontosis, rickettsial infectious disease, protozoal disease, reproduction disease, toothache, pain after tooth extraction, Herpes zoster, Herpes simplex, retroperitoneal fibrosis, or various radiation injuries.

In certain embodiments, the disease is selected from the group consisting of an inflammatory disease, an autoimmune disease, an allergic disorder, and an ocular disorder. In certain embodiments, the disease is selected from the group consisting of pruritus, eczema, asthma, rhinitis, dry eye, ocular inflammation, allergic conjunctivitis, vernal conjunctivitis, vernal keratoconjunctivitis, giant papillary conjunctivitis, fungal keratitis and uveitis.

The method may include modulating the activity of one or more kinases in a subject, such as any of the kinase described above. The method may include inhibiting a kinase. The method may include activating, e.g., stimulating or enhancing the activity of, a kinase. The method may include modulating a single kinase or preferentially modulating a specific kinase over others. The method may include modulating multiple kinases or preferentially modulating two more specific kinases over others.

The method may include providing a compound of the invention. The method may include providing multiple compounds of the invention.

The method may include contacting cells containing a kinase with one or more compounds of the invention. For example and without limitation, contacting a cell with a compound may include exposing a cell to a compound, e.g., in a formulation, such as any of those described above; delivering a compound inside a cell; providing a compound to a subject and allowing a cell in the subject to become exposed to the compound. Contacting may be performed in vivo or in vitro. In vitro contact may include exposure of cells or tissue isolated from a subject. The method may include contacting cells with a single compound of the invention. The method may include contact cells with multiple compounds of the invention.

The method may include administration of a composition to a subject. The compositions may be provided by any suitable route of administration. For example and without limitation, the compositions may be administered buccally, by injection, dermally, enterally, intraarterially, intravenously, intranasally, e.g., by inhalation, intraocularly, orally, parenterally, pulmonarily, rectally, subcutaneously, systemically, topically, e.g., to the skin or eye, transdermally, or with or on an implantable medical device (e.g., stent or drug-eluting stent or balloon equivalents).

The method may include using a composition of the invention to diagnose a disease, disorder, or condition in a subject. For example, a radiolabeled form of a compound may be used a tracer in positron emission tomography (PET) to identify anatomical locations of aberrant kinase activity. PET is known in the art and described in, for example, Wadsak Wolfgang, Mitterhauser Markus (2010), “Basics and principles of radiopharmaceuticals for PET/CT”, European Journal of Radiology, 73 (3): 461-469. doi:10.1016/j.ejrad.2009.12.022; Bailey, D. L; D. W. Townsend; P. E. Valk; M. N. Maisey (2005), Positron Emission Tomography: Basic Sciences. Secaucus, NJ: Springer-Verlag, ISBN 1-85233-798-2; and Carlson, Neil (Jan. 22, 2012). Physiology of Behavior. Methods and Strategies of Research, 11th edition, Pearson, p. 151, ISBN 0205239390, the contents of each of which are incorporated herein by reference. The invention may include administering one or more compositions of the invention for both diagnostic and therapeutic purposes.

### EXAMPLES

### Example 1. General Methods

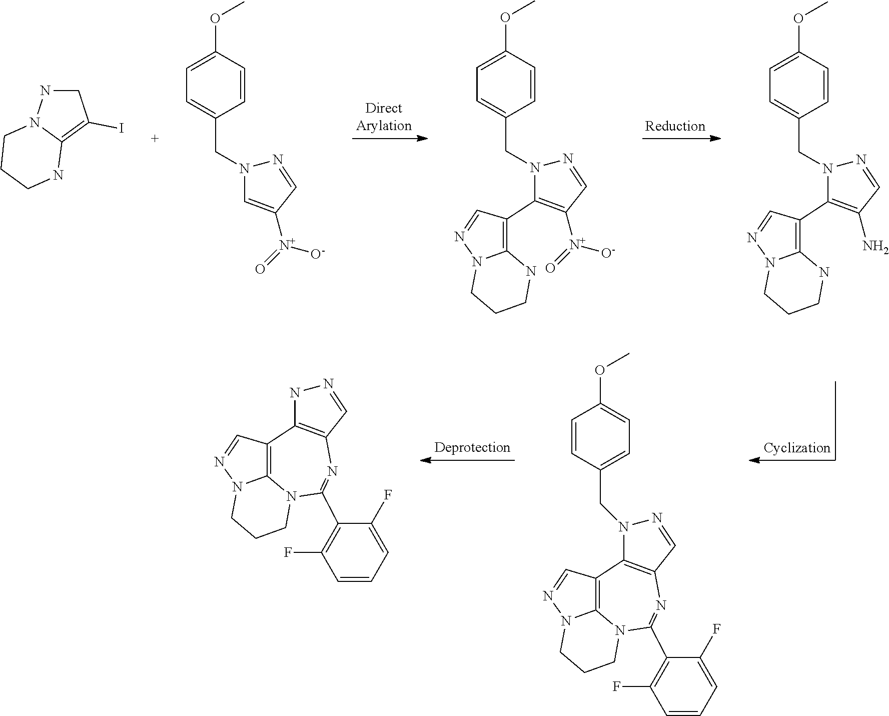

### (1) Direct Arylation Using PdCl₂(PPh₃)₂

3-Iodo-4H,5H,6H,7H-pyrazolo[1,5-a]pyrimidine (500 mg, 2.0 mmol, 1 eq), 1-(4-Methoxybenzyl)-4-nitro-1H-pyrazole (467 mg, 2.0 mmol, 1 eq), pivalic acid (60 mg, 0.6 mmol, 0.3 eq) and K₂CO3 (820 mg, 6.0 mmol, 3 eq) were dissolved in 25 mL dry DMF. The reaction mixture was degassed with N2 under sonication for 5 min, followed by the addition of PdCl₂(PPh₃)₂ 10 mol % (140 mg, 0.2 mmol) and CuI (500 mg, 2.6 mmol, 1.3 eq). The mixture was then degassed again and stirred over night at 120° C. The mixture was filtered over celite and the solids washed with methanol. After concentration under reduced pressure the mixture was diluted with water and extracted with CH₂Cl₂. The combined organic layers were washed with brine, dried over Na₂SO₄, filtered and concentrated under reduced pressure. The crude product was purified by flash chromatography using AcOEt in CyHex to give desired NO₂-intermediate (118 mg, 0.33 mmol, 17% yield).

### (2) Reduction Using Iron

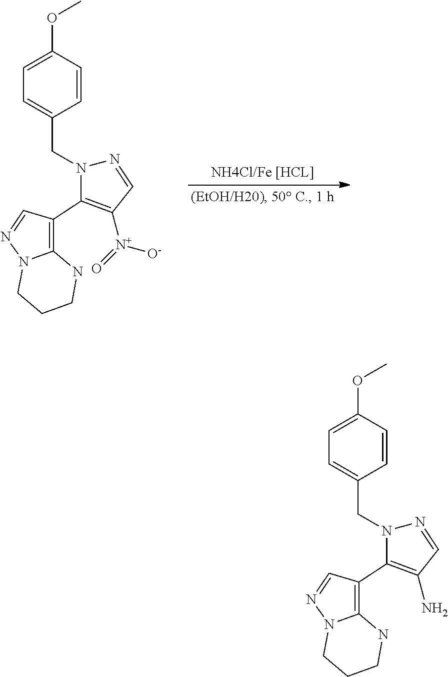 [SMILES: COc1ccc(Cn2ncc([N+](=O)[O-])c2-c2cnn3c2NCCC3)cc1 ; COc1ccc(Cn2ncc(N)c2-c2cnn3c2NCCC3)cc1]

The NO₂—Intermediate (0.33 mmol, 1 eq) was dissolved in 10 mL of ethanol/water 3/1 v/v, iron (170 mg, 3 mmol, 10 eq) and NH₄Cl (164 mg, 3.0 mmol, 10 eq) and some drops HCl 2M were added. The reaction mixture was heated at 50° C. for 1 h. The reaction mixture was filtered over celite, the solids were washed with MeOH and water. The reaction mixture was concentrated under reduced pressure to reduce organic solvent volume. The aqueous layer was extracted with 3×40 mL CH₂Cl₂.

The combined organic layers were washed with brine, dried over Na₂SO₄, filtered and concentrated under reduced pressure. The crude product—NH₂—Intermediate—was used in the next step without further purification.

### (3) Cyclization

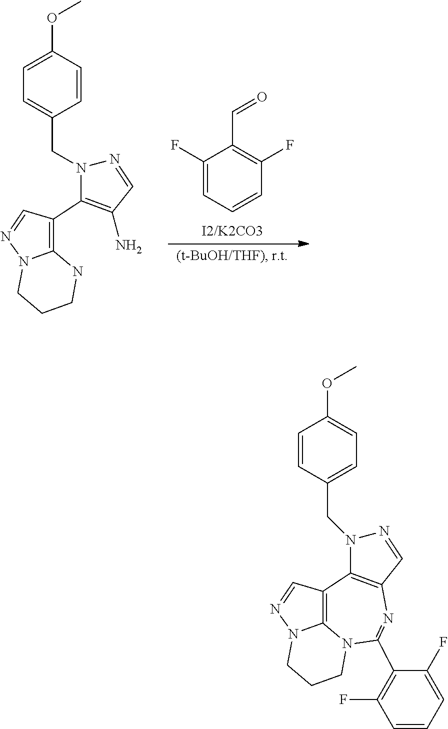 [SMILES: COc1ccc(Cn2ncc(N)c2-c2cnn3c2NCCC3)cc1 ; O=Cc1c(F)cccc1F ; COc1ccc(Cn2ncc3c2-c2cnn4c2N(CCC4)C(c2c(F)cccc2F)=N3)cc1]

The crude NH₂—Intermediate (max 0.33 mmol, 1 eq) was dissolved in 5 mL t-BuOH and some drops of THF, K₂CO3 (140 mg, 1.0 mmol, 3.0 eq) and 2,6-Difluorobenzaldehyde (0.33 mmol, 35 μL, 1.0 eq) were added. After stirring for 30 min at r.t., I₂ (240 mg, 1.0 mmol, 3 eq) was added in one portion and reaction mixture was stirred at r.t. for 1 h. The reaction mixture was quenched with a 5% solution of Na₂S₂O₃ (30 mL) and stirred at r.t. for 30 min. The reaction mixture was concentrated under reduced pressure to reduce organic solvent volume. The aqueous layer was extracted with 3×30 mL CH₂Cl₂. The combined organic layers were washed with brine, dried over Na₂SO₄, filtered and concentrated under reduced pressure. The crude product was used in the next step without further purification.

### (4) PMB-Deprotection

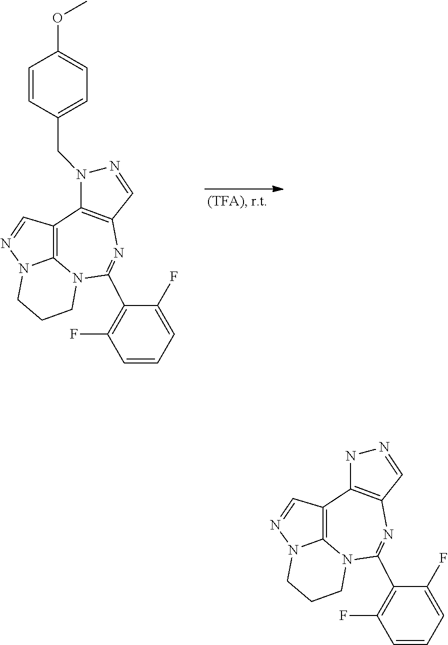 [SMILES: COc1ccc(Cn2ncc3c2-c2cnn4c2N(CCC4)C(c2c(F)cccc2F)=N3)cc1 ; Fc1cccc(F)c1C1=Nc2cn[nH]c2-c2cnn3c2N1CCC3]

Crude cyclized intermediate (max 0.33 mmol) was suspended in TFA (5 mL) and stirred overnight. The reaction mixture was then concentrated under reduced pressure until dryness and then purified via reversed phase Semi Preparative Chromatography using CH₃CN/H₂O to deliver 2.5 mg of desired target molecule.

### Example 2. General Analytical Methods

Method: HPLC-MS F (BG 5-95AB_1.5 min)
col: ‘Agilent Pursit 5 C18 20*2.0 mm’
grad: ‘5%>95%-0.7 min->95%-1.1 min->5%-1.11 min->5%; water (4 L)+TFA (1.5 mL)/acetonitrile (4 L)+TFA (0.75 mL)
flow: ‘1.5 mL/min’
ms: ‘ESI positiv’
Method: HPLC-MS G (BY 10-80CD_7MIN_220&254_Shimadzu)
col: ‘Xbridge Shield RP-18.5 um,2.1*50 mm’
grad: ‘10%>80%-6 min->80%-6.5 min->10%-6.51 min->10%; water (4 L)+NH3·H2O (0.8 mL)/acetonitnile
flow: ‘0.8 mL/min’
ms: ‘ESI positiv’
Method: HPLC-MS H (CF 10-80AB_7MIN_220&254_Shimadzu)
col: ‘Xtimate C18 2.1*30 mm, 3 um’
grad: ‘10%>80%-6 min->80%-6.5 min->10%-6.51 min->10%; water (4 L)+TFA (1.5 mL)/acetonitrile (4 L)+TFA (0.75 mL)
flow: ‘0.8 mL/min’
ms: ‘ESI positiv’
Method: HPLC-MS I (CF 0-60AB_3MIN_220&254_Shimadzu)
col: ‘Xtimate C18 2.1*30 mm, 3 um’
grad: the elution gradient 0%-60% (solvent B) over 1.35 minutes and holding at 60% for 0.9 minutes at a flow rate of 0.8 ml/min; water (4 L)+TFA (1.5 mL)/acetonitrile (4 L)
flow: ‘0.8 mL/min’
ms: ‘ESI positiv’

The products listed in Table 4 were synthesized according to the procedures described in examples 3-70.

TABLE 4

| Compound- | Chemical Structure | HPLC-MS- | Retention | [M + H]⁺ |
|---|---|---|---|---|
| No | (IUPAC NAME) | Method | time (min) | Found |
| Compound 340 | 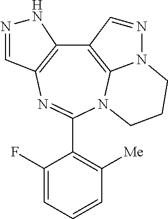 [SMILES: Cc1cccc(F)c1C1=Nc2cn[nH]c2-c2cnn3c2N1CCC3] | HPLC-MS F | 0.587 | 323.2 |
| | 9-(2-fluoro-6-methylphenyl)-3,6,7,8-tetrahydro-2,3,5,5a,8a,10 | | | |
| | hexaazabenzo[cd]cyclopenta[h]azulene | | | |
| Compound 341 | 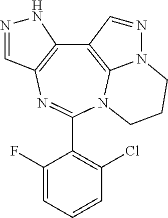 [SMILES: Fc1cccc(Cl)c1C1=Nc2cn[nH]c2-c2cnn3c2N1CCC3] | HPLC-MS F | 0.590 | 343.2 |
| | 9-(2-chloro-6-fluorophenyl)-3,6,7,8-tetrahydro-2,3,5,5a,8a,10- | | | |
| | hexaazabenzo[cd]cyclopenta[h]azulene | | | |
| Compound 342 | 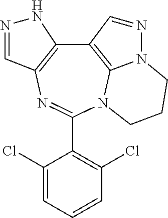 [SMILES: Clc1cccc(Cl)c1C1=Nc2cn[nH]c2-c2cnn3c2N1CCC3] | HPLC-MS F | 0.648 | 359.1 |
| | 9-(2,6-dichlorophenyl)-3,6,7,8-tetrahydro-2,3,5,5a,8a,10- | | | |
| | hexaazabenzo[cd]cyclopenta[h]azulene | | | |
| Compound 343 | 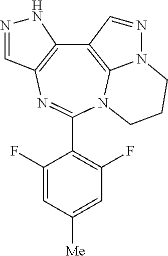 [SMILES: Cc1cc(F)c(C2=Nc3cn[nH]c3-c3cnn4c3N2CCC4)c(F)c1] | HPLC-MS F | 0.631 | 341.1 |
| | 9-(2,6-difluoro-4-methylphenyl)-3,6,7,8-tetrahydro-2,3,5,5a,8a,10 | | | |
| | hexaazabenzo[cd]cyclopenta[h]azulene | | | |
| Compound 344 | 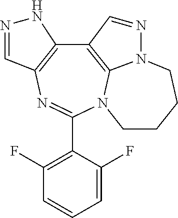 [SMILES: Fc1cccc(F)c1C1=Nc2cn[nH]c2-c2cnn3c2N1CCCC3] | HPLC-MS F | 0.732 | 341.1 |
| | 10-(2,6-difluorophenyl)-6,7,8,9-tetrahydro-3H-2,3,5,5a,9a,11- | | | |
| | hexaazadicyclopenta[b,kl]heptalene | | | |
| Compound 345 | 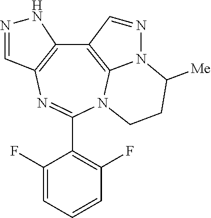 [SMILES: CC1CCN2C(c3c(F)cccc3F)=Nc3cn[nH]c3-c3cnn1c32] | HPLC-MS F | 0.698 | 341.1 |
| | 9-(2,6-difluorophenyl)-6-methyl-3,6,7,8-tetrahydro- | | | |
| | 2,3,5,5a,8a,10-hexaazabenzo[cd]cyclopenta[h]azulene | | | |
| Compound 346 | 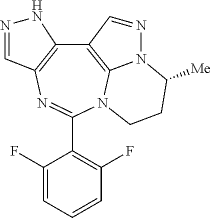 [SMILES: C[C@@H]1CCN2C(c3c(F)cccc3F)=Nc3cn[nH]c3-c3cnn1c32] | HPLC-MS F | 0.712 | 341.1 |
| | (R)-9-(2,6-difluorophenyl)-6-methyl-3,6,7,8-tetrahydro- | | | |
| | 2,3,5,5a,8a,10-hexaazabenzo[cd]cyclopenta[h]azulene | | | |
| Compound 347 | 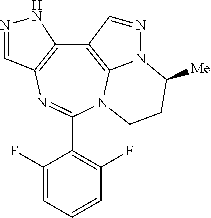 [SMILES: C[C@H]1CCN2C(c3c(F)cccc3F)=Nc3cn[nH]c3-c3cnn1c32] | HPLC-MS F | 0.712 | 341.1 |
| | (S)-9-(2,6-difluorophenyl)-6-methyl-3,6,7,8-tetrahydro- | | | |
| | 2,3,5,5a,8a,10-hexaazabenzo[cd]cyclopenta[h]azulene | | | |
| Compound 348 | 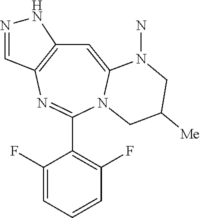 [SMILES: CC1CN(N)C2=Cc3[nH]ncc3N=C(c3c(F)cccc3F)N2C1] | HPLC-MS F | 0.692 | 341.1 |
| | 9-(2,6-difluorophenyl)-7-methyl-2,6,7,8-tetrahydro- | | | |
| | 2,3,5,5a,8a,10-hexaazabenzo[cd]cyclopenta[h]azulene | | | |
| Compound 349 | 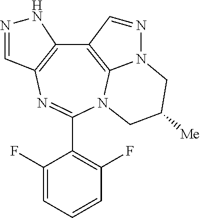 [SMILES: C[C@@H]1CN2C(c3c(F)cccc3F)=Nc3cn[nH]c3-c3cnn(c32)C1] | HPLC-MS F | 0.702 | 341.1 |
| | (11R)-8-(2,6-difluorophenyl)-11-methyl-3,4,7,9,13,14- | | | |
| | hexazatetracyclo[7.6.1.02,6.013,16]hexadeca- | | | |
| | 1(16),2(6),4,7,14-pentaene | | | |
| Compound 350 | 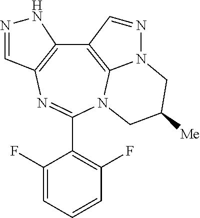 [SMILES: C[C@H]1CN2C(c3c(F)cccc3F)=Nc3cn[nH]c3-c3cnn(c32)C1] | HPLC-MS F | 0.702 | 341.1 |
| | (11S)-8-(2,6-difluorophenyl)-11-methyl-3,4,7,9,13,14- | | | |
| | hexazatetracyclo[7.6.1.02,6.013,16]hexadeca- | | | |
| | 1(16),2(6),4,7,14-pentaene | | | |
| Compound 351 | 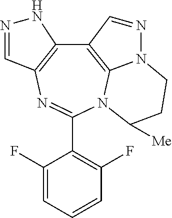 [SMILES: CC1CCn2ncc3c2N1C(c1c(F)cccc1F)=Nc1cn[nH]c1-3] | HPLC-MS F | 0.695 | 341.1 |
| | 9-(2,6-difluorophenyl)-8-methyl-3,6,7,8-tetrahydro- | | | |
| | 2,3,5,5a,8a,10-hexaazabenzo[cd]cyclopenta[h]azulene | | | |
| Compound 352 | 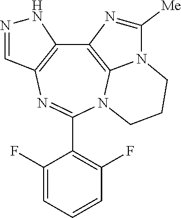 [SMILES: Cc1nc2c3n1CCCN3C(c1c(F)cccc1F)=Nc1cn[nH]c1-2] | HPLC-MS H | 0.943 | 341.1 |
| | 8-(2,6-difluorophenyl)-3,4,7,9,13,14- | | | |
| | hexazatetracyclo[7.6.1.02,6.013,16]hexadeca- | | | |
| | 1(16),2(6),4,7,14-pentaene | | | |
| Compound 353 | 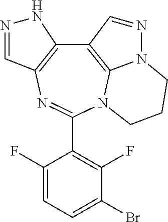 [SMILES: Fc1ccc(Br)c(F)c1C1=Nc2cn[nH]c2-c2cnn3c2N1CCC3] | HPLC-MS F | 0.746 | 407.0 |
| | 9-(3-bromo-2,6-difluorophenyl)-3,6,7,8-tetrahydro- | | | |
| | 2,3,5,5a,8a,10-hexaazabenzo[cd]cyclopenta[h]azulene | | | |
| Compound 354 | 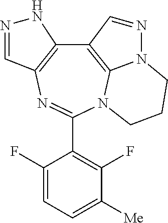 [SMILES: Cc1ccc(F)c(C2=Nc3cn[nH]c3-c3cnn4c3N2CCC4)c1F] | HPLC-MS F | 0.701 | 341.1 |
| | 9-(2,6-difluoro-3-methylphenyl)-3,6,7,8-tetrahydro-2,3,5,5a,8a,10 | | | |
| | Hexaazabenzo[cd]cyclopenta[h]azulene | | | |
| Compound 355 | 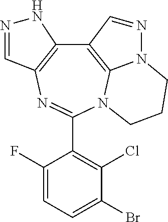 [SMILES: Fc1ccc(Br)c(Cl)c1C1=Nc2cn[nH]c2-c2cnn3c2N1CCC3] | HPLC-MS F | 0.793 | 423 |
| | 8-(3-bromo-2-chloro-6-fluoro-phenyl)-3,4,7,9,13,14- | | | |
| | hexazatetracyclo-[7.6.1.02,6.013,16]hexadeca- | | | |
| | 1(16),2(6),4,7,14-pentaene | | | |
| Compound 356 | 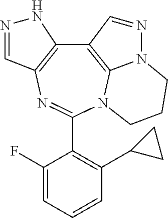 [SMILES: Fc1cccc(C2CC2)c1C1=Nc2cn[nH]c2-c2cnn3c2N1CCC3] | HPLC-MS I | 0.700 | 349.1 |
| | 9-(2-cyclopropyl-6-fluorophenyl)-3,6,7,8-tetrahydro- | | | |
| | 2,3,5,5a,8a,10-hexaazabenzo[cd]cyclopenta[h]azulene | | | |
| Compound 357 | 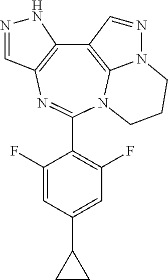 [SMILES: Fc1cc(C2CC2)cc(F)c1C1=Nc2cn[nH]c2-c2cnn3c2N1CCC3] | HPLC-MS H | 0.735 | 341.1 |
| | 9-(2,6-difluorophenyl)-7-methyl-2,6,7,8-tetrahydro- | | | |
| | 2,3,5,5a,8a,10-hexaazabenzo[cd]cyclopenta[h]azulene | | | |
| Compound 358 | 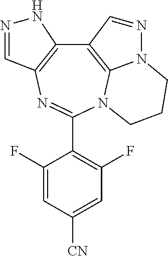 [SMILES: N#Cc1cc(F)c(C2=Nc3cn[nH]c3-c3cnn4c3N2CCC4)c(F)c1] | HPLC-MS F | 0.707 | 352.1 |
| | 3,5-difluoro-4-[3-[(4-methoxyphenyl)methyl]-3,4,7,9,13,14- | | | |
| | hexazatetracyclo[7.6.1.02,6.013,16]hexadeca- | | | |
| | 1(16),2(6),4,7,14-pentaen-8-yl]benzonitrile | | | |
| Compound 359 | 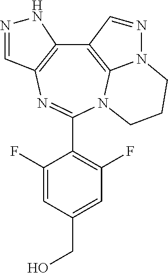 [SMILES: OCc1cc(F)c(C2=Nc3cn[nH]c3-c3cnn4c3N2CCC4)c(F)c1] | HPLC-MS F | 0.626 | 357.1 |
| | (3,5-difluoro-4-(3,6,7,8-tetrahydro-2,3,5,5a,8a,10- | | | |
| | hexaazabenzo[cd]cyclopenta[h]azulen-9-yl)phenyl)methanol | | | |
| Compound 360 | 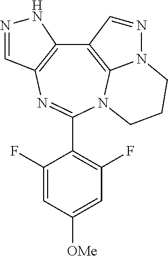 [SMILES: COc1cc(F)c(C2=Nc3cn[nH]c3-c3cnn4c3N2CCC4)c(F)c1] | HPLC-MS F | 0.615 | 357.2 |
| | 8-(2,6-difluoro-4-methoxy-phenyl)-3,4,7,9,13,14- | | | |
| | hexazatetracyclo[7.6.1.02,6.013,16]hexadeca- | | | |
| | 1(16),2(6),4,7,14-pentaene | | | |
| Compound 361 | 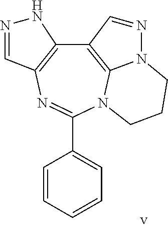 [SMILES: c1ccc(C2=Nc3cn[nH]c3-c3cnn4c3N2CCC4)cc1] | HPLC-MS F | 0.522 | 291.1 |
| | 8-phenyl-3,4,7,9,13,14-hexazatetracyclo[7.6.1.02,6.013,16] | | | |
| | hexadeca-1(16),2(6),4,7,14-pentaene | | | |
| Compound 362 | 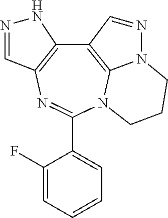 [SMILES: Fc1ccccc1C1=Nc2cn[nH]c2-c2cnn3c2N1CCC3] | HPLC-MS F | 0.606 | 309.1 |
| | 9-(2-fluorophenyl)-3,6,7,8-tetrahydro-2,3,5,5a,8a,10- | | | |
| | hexaazabenzo[cd]cyclopenta[h]azulene | | | |
| Compound 363 | 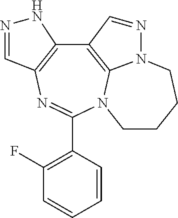 [SMILES: Fc1ccccc1C1=Nc2cn[nH]c2-c2cnn3c2N1CCCC3] | HPLC-MS F | 0.695 | 323.1 |
| | 10-(2-fluorophenyl)-6,7,8,9-tetrahydro-3H-2,3,5,5a,9a,11- | | | |
| | hexaazadicyclopenta[b,kl]heptalene | | | |
| Compound 364 | 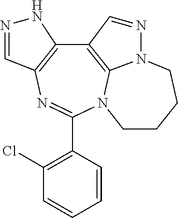 [SMILES: Clc1ccccc1C1=Nc2cn[nH]c2-c2cnn3c2N1CCCC3] | HPLC-MS F | 0.676 | 338.9 |
| | 10-(2-chlorophenyl)-6,7,8,9-tetrahydro-3H-2,3,5,5a,9a,11- | | | |
| | hexaazadicyclopenta[b,kl]heptalene | | | |
| Compound 365 | 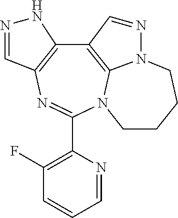 [SMILES: Fc1cccnc1C1=Nc2cn[nH]c2-c2cnn3c2N1CCCC3] | HPLC-MS F | 0.118 | 342.1 |
| | 10-(3-fluoropyridin-2-yl)-6,7,8,9-tetrahydro-3H- | | | |
| | 2,3,5,5a,9a,11-hexaazadicyclopenta[b,kl]heptalene | | | |
| Compound 366 | 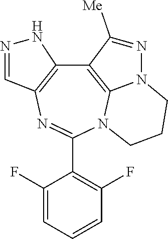 [SMILES: Cc1nn2c3c1-c1[nH]ncc1N=C(c1c(F)cccc1F)N3CCC2] | HPLC-MS F | 0.722 | 341.1 |
| | 9-(2,6-difluorophenyl)-4-methyl-3,6,7,8-tetrahydro- | | | |
| | 2,3,5,5a,8a,10-hexaazabenzo[cd]cyclopenta[h]azulene | | | |
| Compound 367 | 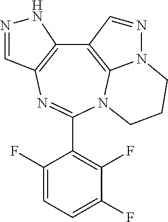 [SMILES: Fc1ccc(F)c(C2=Nc3cn[nH]c3-c3cnn4c3N2CCC4)c1F] | HPLC-MS F | 0.704 | 345.1 |
| | 8-(2,3,6-trifluorophenyl)-3,4,7,9,13,14- | | | |
| | hexazatetracyclo[7.6.1.02,6.013,16]hexadeca- | | | |
| | 1(16),2(6),4,7,14-pentaene | | | |
| Compound 368 | 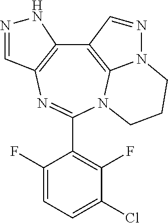 [SMILES: Fc1ccc(Cl)c(F)c1C1=Nc2cn[nH]c2-c2cnn3c2N1CCC3] | HPLC-MS F | 0.729 | 361.1 |
| | 8-(3-chloro-2,6-difluoro-phenyl)-3,4,7,9,13,14- | | | |
| | hexazatetracyclo[7.6.1.02,6.013,16]hexadeca- | | | |
| | 1(16),2(6),4,7,14-pentaene | | | |
| Compound 369 | 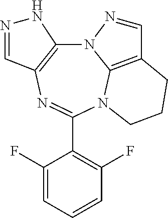 [SMILES: Fc1cccc(F)c1C1=Nc2cn[nH]c2-n2ncc3c2N1CCC3] | HPLC-MS F | 0.773 | 327.0 |
| | 9-(2,6-difluorophenyl)-3,6,7,8-tetrahydro-2,3,3b,4,8a,10- | | | |
| | hexaazabenzo[cd]cyclopenta[h]azulene | | | |
| Compound 370 | 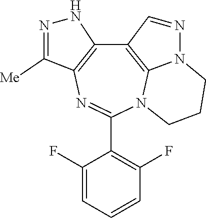 [SMILES: Cc1n[nH]c2c1N=C(c1c(F)cccc1F)N1CCCn3ncc-2c31] | HPLC-MS F | 0.595 | 341.1 |
| | 9-(2,6-difluorophenyl)-1-methyl-3,6,7,8-tetrahydro- | | | |
| | 2,3,5,5a,8a,10-hexaazabenzo[cd]cyclopenta[h]azulene | | | |
| Compound 371 | 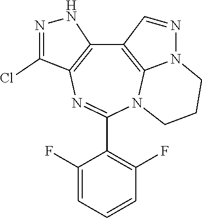 [SMILES: Fc1cccc(F)c1C1=Nc2c(Cl)n[nH]c2-c2cnn3c2N1CCC3] | HPLC-MS F | 0.764 | 361.0 |
| | 5-chloro-8-(2,6-difluorophenyl)-3,4,7,9,13,14- | | | |
| | hexazatetracyclo[7.6.1.02,6.013,16]hexadeca- | | | |
| | 1(16),2(6),4,7,14-pentaene | | | |
| Compound 372 | 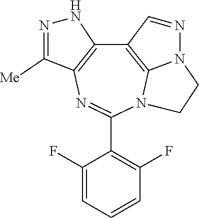 [SMILES: Cc1n[nH]c2c1N=C(c1c(F)cccc1F)N1CCn3ncc-2c31] | HPLC-MS F | 0.422 | 327.0 |
| | 8-(2,6-difluorophenyl)-5-methyl-3,4,7,9,12,13- | | | |
| | hexazatetrahydro[7.5.1.02,6.012,15]pentadeca- | | | |
| | 1(15),2,5,7,13-pentaene | | | |
| Compound 373 | 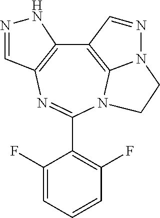 [SMILES: Fc1cccc(F)c1C1=Nc2cn[nH]c2-c2cnn3c2N1CC3] | HPLC-MS F | 0.64 | 313.1 |
| | 3-(2,6-difluorophenyl)-2,7-dihydro-1H-2a,4,6,7,9,9a- | | | |
| | hexaazadicyclopenta[cd,f]azulene | | | |
| Compound 374 | 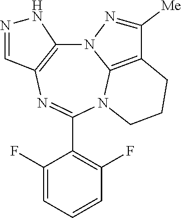 [SMILES: Cc1nn2c3c1CCCN3C(c1c(F)cccc1F)=Nc1cn[nH]c1-2] | HPLC-MS F | 0.79 | 341.1 |
| | 8-(2,6-difluorophenyl)-14-methyl-1,3,4,7,9,15- | | | |
| | hexazatetracyclo[7.6.1.02,6.013,16]hexadeca- | | | |
| | 2(6),4,7,13(16),14-pentaene | | | |
| Compound 375 | 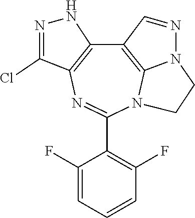 [SMILES: Fc1cccc(F)c1C1=Nc2c(Cl)n[nH]c2-c2cnn3c2N1CC3] | HPLC-MS F | 0.79 | 347.1 |
| | 5-chloro-3-(2,6-difluorophenyl)-2,7-dihydro-1H- | | | |
| | 2a,4,6,7,9,9a-hexaazadicyclopenta[cd,f]azulene | | | |
| Compound 376 | 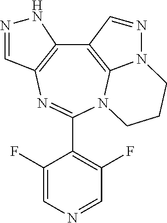 [SMILES: Fc1cncc(F)c1C1=Nc2cn[nH]c2-c2cnn3c2N1CCC3] | HPLC-MS F | 0.66 | 328.1 |
| | 8-(3,5-difluoro-4-pyridyl)-3,4,7,9,13,14- | | | |
| | hexazatetracyclo[7.6.1.02,6.013,16]hexadeca- | | | |
| | 1(16),2(6),4,7,14-pentaene | | | |
| Compound 377 | 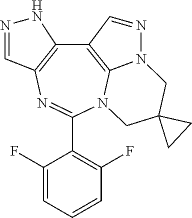 [SMILES: Fc1cccc(F)c1C1=Nc2cn[nH]c2-c2cnn3c2N1CC1(CC1)C3] | HPLC-MS F | 0.76 | 353.1 |
| | 9-(2,6-difluorophenyl)-6,8-dihydro-3H-spiro[2,3,5,5a,8a,10- | | | |
| | hexaazabenzo[cd]cyclopenta[h]azulene-7,1′-cyclopropane] | | | |
| Compound 379 | 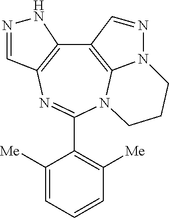 [SMILES: Cc1cccc(C)c1C1=Nc2cn[nH]c2-c2cnn3c2N1CCC3] | HPLC-MS F | 0.68 | 319.1 |
| | 8-(2,6-dimethylphenyl)-3,4,7,9,13,14- | | | |
| | hexazatetracyclo[7.6.1.02,6.013,16]hexadeca- | | | |
| | 1(16),2(6),4,7,14-pentaene | | | |
| Compound 380 | 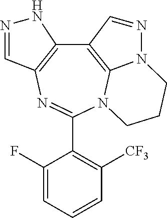 [SMILES: Fc1cccc(C(F)(F)F)c1C1=Nc2cn[nH]c2-c2cnn3c2N1CCC3] | HPLC-MS F | 0.77 | 376.9 |
| | 8-[2-fluoro-6-(trifluoromethyl)phenyl]-3,4,7,9,13,14- | | | |
| | hexazatetracyclo[7.6.1.02,6.013,16]hexadeca- | | | |
| | 1(16),2(6),4,7,14-pentaene | | | |
| Compound 381 | 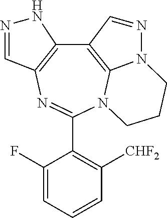 [SMILES: Fc1cccc(C(F)F)c1C1=Nc2cn[nH]c2-c2cnn3c2N1CCC3] | LCMS-I 10- 80AB_2min_ 220&254_Shimadzu.lcm | 0.85 | 359.2 |
| | 8-[2-(difluoromethyl)-6-fluoro-phenyl]-3,4,7,9,13,14- | | | |
| | hexazatetracyclo[7.6.1.02,6.013,16]hexadeca- | | | |
| | 1(16),2(6),4,7,14-pentaene | | | |
| Compound 382 | 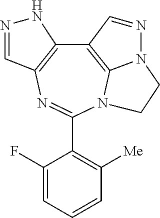 [SMILES: Cc1cccc(F)c1C1=Nc2cn[nH]c2-c2cnn3c2N1CC3] | HPLC-MS F | 0.63 | 309.1 |
| | 8-(2-fluoro-6-methyl-phenyl)-3,4,7,9,12,13- | | | |
| | hexazatetracyclo[7.5.1.02,6.012,15]pentadeca- | | | |
| | 1(15),2(6),4,7,13-pentaene | | | |
| Compound 383 | 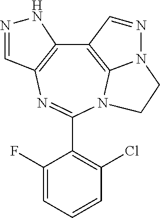 [SMILES: Fc1cccc(Cl)c1C1=Nc2cn[nH]c2-c2cnn3c2N1CC3] | HPLC-MS F | 0.67 | 329.0 |
| | 8-(2-chloro-6-fluoro-phenyl)-3,4,7,9,12,13- | | | |
| | hexazatetracyclo[7.5.1.02,6.012,15]pentadec- | | | |
| | 1(15),2(6),4,7,13-pentaen | | | |
| Compound 384 | 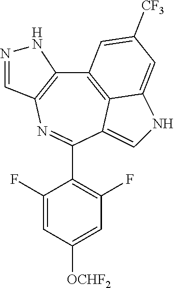 [SMILES: Fc1cc(OC(F)F)cc(F)c1C1=Nc2cn[nH]c2-c2cc(C(F)(F)F)cc3[nH]cc1c23] | HPLC-MS F | 0.85 | 455.0 |
| | 3-(4-(difluoromethoxy)-2,6-difluorophenyl)-9-(trifluoromethyl)- | | | |
| | 1,7-dihydropyrazolo[3′,4′:6,7]azepino[3,4,5-cd]indole | | | |
| Compound 385 | 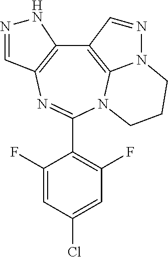 [SMILES: Fc1cc(Cl)cc(F)c1C1=Nc2cn[nH]c2-c2cnn3c2N1CCC3] | HPLC-MS F | 0.78 | 361.0 |
| | 9-(4-chloro-2,6-difluorophenyl)-3,6,7,8-tetrahydro- | | | |
| | 2,3,5,5a,8a,10-hexaazabenzo[cd]cyclopenta[h]azulene | | | |
| Compound 386 | 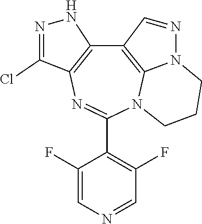 [SMILES: Fc1cncc(F)c1C1=Nc2c(Cl)n[nH]c2-c2cnn3c2N1CCC3] | HPLC-MS F | 0.78 | 362.1 |
| | 1-chloro-9-(3,5-difluoropyridin-4-yl)-3,6,7,8-tetrahydro- | | | |
| | 2,3,5,5a,8a,10-hexaazabenzo[cd]cyclopenta[h]azulene | | | |
| Compound 387 | 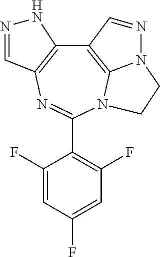 [SMILES: Fc1cc(F)c(C2=Nc3cn[nH]c3-c3cnn4c3N2CC4)c(F)c1] | HPLC-MS F | 0.66 | 331.0 |
| | 3-(2,4,6-trifluorophenyl)-2,7-dihydro-1H-2a,4,6,7,9,9a- | | | |
| | hexaazadicyclopenta[cd,f]azulene | | | |
| Compound 388 | 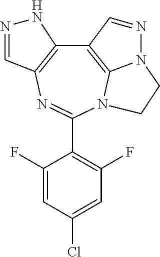 [SMILES: Fc1cc(Cl)cc(F)c1C1=Nc2cn[nH]c2-c2cnn3c2N1CC3] | HPLC-MS F | 0.64 | 346.9 |
| | 3-(4-chloro-2,6-difluorophenyl)-2,7-dihydro-1H- | | | |
| | 2a,4,6,7,9,9a-hexaazadicyclopenta[cd,f]azulene | | | |
| Compound 389 | 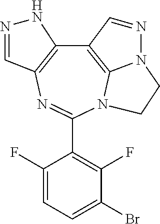 [SMILES: Fc1ccc(Br)c(F)c1C1=Nc2cn[nH]c2-c2cnn3c2N1CC3] | HPLC-MS F | 0.74 | 391.0 |
| | 3-(3-bromo-2,6-difluorophenyl)-2,7-dihydro-1H- | | | |
| | 2a,4,6,7,9,9a-hexaazadicyclopenta[cd,f]azulene | | | |
| Compound 390 | 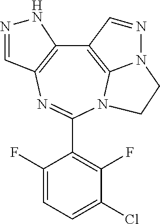 [SMILES: Fc1ccc(Cl)c(F)c1C1=Nc2cn[nH]c2-c2cnn3c2N1CC3] | HPLC-MS F | 0.66 | 346.9 |
| | 3-(3-chloro-2,6-difluorophenyl)-2,7-dihydro-1H- | | | |
| | 2a,4,6,7,9,9a-hexaazadicyclopenta[cd,f]azulene | | | |
| Compound 391 | 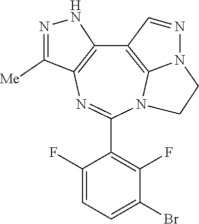 [SMILES: Cc1n[nH]c2c1N=C(c1c(F)ccc(Br)c1F)N1CCn3ncc-2c31] | HPLC-MS F | 0.72 | 406.9 |
| | 3-(3-bromo-2,6-difluorophenyl)-5-methyl-2,7-dihydro-1H- | | | |
| | 2a,4,6,7,9,9a-hexaazadicyclopenta[cd,f]azulene | | | |
| Compound 392 | 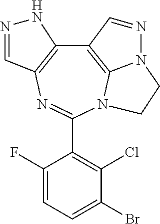 [SMILES: Fc1ccc(Br)c(Cl)c1C1=Nc2cn[nH]c2-c2cnn3c2N1CC3] | HPLC-MS G | 2.47 | 409.1 |
| | 3-(3-bromo-2-chloro-6-fluorophenyl)-2,7-dihydro-1H- | | | |
| | 2a,4,6,7,9,9a-hexaazadicyclopenta[cd,f]azulene | | | |
| Compound 393 | 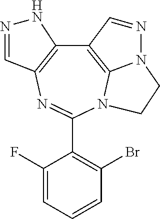 [SMILES: Fc1cccc(Br)c1C1=Nc2cn[nH]c2-c2cnn3c2N1CC3] | HPLC-MS F | 0.67 | 375.0 |
| | 3-(2-bromo-6-fluorophenyl)-2,7-dihydro-1H-2a,4,6,7,9,9a- | | | |
| | hexaazadicyclopenta[cd,f]azulene | | | |
| Compound 394 | 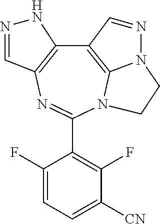 [SMILES: N#Cc1ccc(F)c(C2=Nc3cn[nH]c3-c3cnn4c3N2CC4)c1F] | HPLC-MS F | 0.61 | 337.9 |
| | 3-(2,7-dihydro-1H-2a,4,6,7,9,9a-hexaazadicyclopenta | | | |
| | [cd,f]azulen-3-yl)-2,4-difluorobenzonitrile | | | |
| Compound 395 | 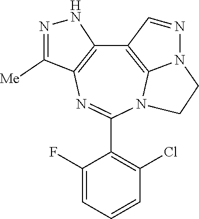 [SMILES: Cc1n[nH]c2c1N=C(c1c(F)cccc1Cl)N1CCn3ncc-2c31] | HPLC-MS F | 0.73 | 343.1 |
| | 3-(2-chloro-6-fluorophenyl)-5-methyl-2,7-dihydro-1H- | | | |
| | 2a,4,6,7,9,9a-hexaazadicyclopenta[cd,f]azulene | | | |
| Compound 396 | 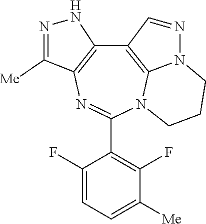 [SMILES: Cc1ccc(F)c(C2=Nc3c(C)n[nH]c3-c3cnn4c3N2CCC4)c1F] | HPLC-MS F | 0.80 | 355.1 |
| | 9-(2,6-difluoro-3-methylphenyl)-1-methyl-3,6,7,8-tetrahydro- | | | |
| | 2,3,5,5a,8a,10-hexaazabenzo[cd]cyclopenta[h]azulene | | | |
| Compound 397 | 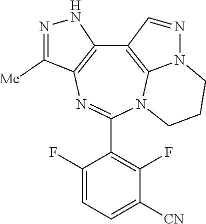 [SMILES: Cc1n[nH]c2c1N=C(c1c(F)ccc(C#N)c1F)N1CCCn3ncc-2c31] | HPLC-MS F | 0.78 | 366.0 |
| | 2,4-difluoro-3-(1-methyl-3,6,7,8-tetrahydro-2,3,5,5a,8a,10- | | | |
| | hexaazabenzo[cd]cyclopenta[h]azulen-9-yl)benzonitrile | | | |
| Compound 398 | 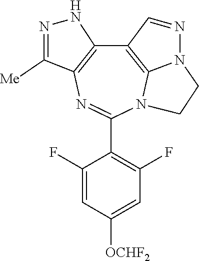 [SMILES: Cc1n[nH]c2c1N=C(c1c(F)cc(OC(F)F)cc1F)N1CCn3ncc-2c31] | HPLC-MS F | 0.78 | 393.1 |
| | 3-(4-difluoromethoxy)-2,6-difluorophenyl)-5-methyl-2,7- | | | |
| | dihydro-1H-2a,4,6,7,9,9a-hexaazadicyclopenta[cd,f]azulene | | | |
| Compound 399 | 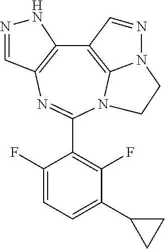 [SMILES: Fc1ccc(C2CC2)c(F)c1C1=Nc2cn[nH]c2-c2cnn3c2N1CC3] | HPLC-MS F | 0.66 | 353.0 |
| | 3-(3-cyclopropyl-2,6-difluorophenyl)-2,7-dihydro-1H- | | | |
| | 2a,4,6,7,9,9a-hexaazadicyclopenta[cd,f]azulene | | | |
| Compound 400 | 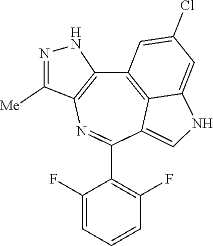 [SMILES: Cc1n[nH]c2c1N=C(c1c(F)cccc1F)c1c[nH]c3cc(Cl)cc-2c13] | HPLC-MS F | 0.81 | 369.0 |
| | 9-chloro-3-(2,6-difluorophenyl)-5-methyl-1,7- | | | |
| | dihydropyrazolo[3′,4′:6,7]azepino[3,4,5-cd]indole | | | |
| Compound 401 | 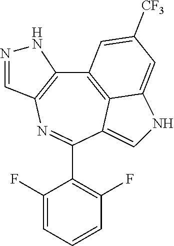 [SMILES: Fc1cccc(F)c1C1=Nc2cn[nH]c2-c2cc(C(F)(F)F)cc3[nH]cc1c23] | BK0- 80AB_3min_ 220&254_Shimadzu | 1.31 | 389.2 |
| | 3-(2,6-difluorophenyl)-9-(trifluoromethyl)-1,7- | | | |
| | dihydropyrazolo[3′,4′:6,7]azepino[3,4,5-cd]indole | | | |
| Compound 402 | 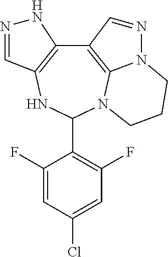 [SMILES: Fc1cc(Cl)cc(F)c1C1Nc2cn[nH]c2-c2cnn3c2N1CCC3] | HPLC-MS G | 3.53 | 363.0 |
| | 9-(4-chloro-2,6-difluorophenyl)-3,6,7,8,9,10-hexahydro- | | | |
| | 2,3,5,5a,8a,10-hexaazabenzo[cd]cyclopenta[h]azulene | | | |
| Compound 403 | 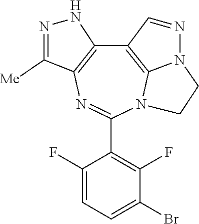 [SMILES: Cc1n[nH]c2c1N=C(c1c(F)ccc(Br)c1F)N1CCn3ncc-2c31] | HPLC-MS F | 0.81 | 421.0 |
| | 3-(3-bromo-2,6-difluorophenyl)-5-methyl-2,7-dihydro-1H- | | | |
| | 2a,4,6,7,9,9a-hexaazadicyclopenta[cd,f]azulene | | | |
| Compound 404 | 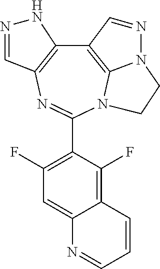 [SMILES: Fc1cc2ncccc2c(F)c1C1=Nc2cn[nH]c2-c2cnn3c2N1CC3] | HPLC-MS F | 0.68 | 364.1 |
| | 3-(5,7-difluoroquinolin-6-yl)-2,7-dihydro-1H-2a,4,6,7,9,9a- | | | |
| | hexaazadicyclopenta[cd,f]azulene | | | |
| Compound 405 | 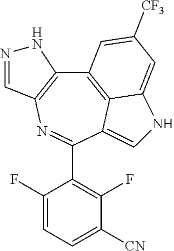 [SMILES: N#Cc1ccc(F)c(C2=Nc3cn[nH]c3-c3cc(C(F)(F)F)cc4[nH]cc2c34)c1F] | HPLC-MS F | 0.85 | 414.0 |
| | 2,4-difluoro-3-(9-(trifluoromethyl)-1,7-dihydropyrazolo | | | |
| | [3′,4′:6,7]azepino[3,4,5-cd]indol-3-yl)benzonitrile | | | |
| Compound 406 | 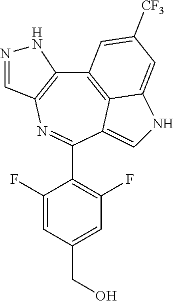 [SMILES: OCc1cc(F)c(C2=Nc3cn[nH]c3-c3cc(C(F)(F)F)cc4[nH]cc2c34)c(F)c1] | HPLC-MS F | 0.77 | 419.1 |
| | (3,5-difluoro-4-(9-(trifluoromethyl)-1,7- | | | |
| | dihydropyrazolo[3′,4′:6,7]azepino[3,4,5-cd]indol-3- | | | |
| | yl)phenyl)methanol | | | |
| Compound 407 | 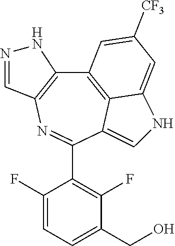 [SMILES: OCc1ccc(F)c(C2=Nc3cn[nH]c3-c3cc(C(F)(F)F)cc4[nH]cc2c34)c1F] | HPLC-MS F | 0.77 | 419.1 |
| | (2,4-difluoro-3-(9-(trifluoromethyl)-1,7- | | | |
| | dihydropyrazolo[3′,4′:6,7]azepino[3,4,5-cd]indol-3- | | | |
| | yl)phenyl)methanol | | | |
| Compound 408 | 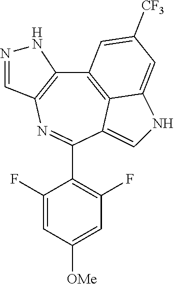 [SMILES: COc1cc(F)c(C2=Nc3cn[nH]c3-c3cc(C(F)(F)F)cc4[nH]cc2c34)c(F)c1] | HPLC-MS F | 0.84 | 419.0 |
| | 3-(2,6-difluoro-1-methoxyphenyl)-9-(trifluoromethyl)-1,7- | | | |
| | dihydropyrazolo[3′,4′:6,7]azepino[3,4,5-cd]indole | | | |

### Example 4. 9-(2-fluoro-6-methylphenyl)-3,6,7,8-tetrahydro-2,3,5,5a,8a,10 hexaazabenzo[cd]cyclopenta[h]azulene (Compound 340)

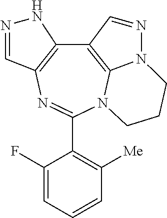 [SMILES: Cc1cccc(F)c1C1=Nc2cn[nH]c2-c2cnn3c2N1CCC3]

### Step 1. 1-(4-methoxybenzyl)-4-nitro-1H-pyrazole (340-a)

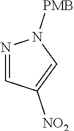

To a solution of 4-nitro-1H-pyrazole (45 g, 0.398 mol) in CH₃CN (500 mL) was added K₂CO₃ (82.5 g, 0.60 mol) and 1-(chloromethyl)-4-methoxybenzene (68.56 g, 0.438 mol). The mixture was heated to 55° C. and stirred for 4 hr. The reaction mixture was concentrated under reduced pressure to remove CH₃CN (500 mL). The residue was diluted with H₂O (200 mL) and extracted with EtOAc (200 mL×2). The combined organic phase was washed with brine (200 mL), dried with anhydrous Na₂SO₄, filtered and concentrated in vacuum. The crude product was purified by flash chromatography on silica gel column using 0-100% EtOAc/petroleum ether as eluent, to provide the title product 340-a as a light yellow solid (90 g, 97.0%).

### Step 2. 4,5,6,7-tetrahydropyrazolo[1,5-a]pyrimidine (340-b)

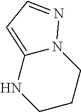

To a solution of 1H-pyrazol-5-amine (30 g, 0.361 mol) and TEA (109.6 g, 1.08 mol) in dioxane (1 L) was added 1,3-dibromopropane (72.89 g, 0.361 mol). The mixture was heated to 110° C. and stirred for 5 hr. The reaction mixture was filtered. The filtration was concentrated to dryness. The crude product was purified by flash chromatography on silica gel column using 0-1% MeOH/DCM as eluent, to provide the title product 340-b as a pale-yellow solid (36.4 g, 40.9%).

### Step 3. tert-butyl 6,7-dihydropyrazolo[1,5-a]pyrimidine-4(5H)-carboxylate (340-c)

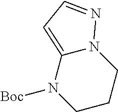 [SMILES: CC(C)(C)OC(=O)N1CCCn2nccc21]

To a solution of 340-b (16 g, 0.130 mmol) in THE (400 mL) was added NaH (7.79 g, 0.195 mol, 60 wt % in mineral oil) at 0-5° C. The reaction was stirred at 20° C. for 1 hour under N₂ atmosphere. Then tertbutoxycarbonyl tert-butyl carbonate (42.53 g, 0.195 mol) was added and the mixture was stirred at 20° C. for 16 hr. The reaction mixture was poured into water (400 mL) and extracted with EtOAc (100 mL×2). The organic layer was washed with brine (100 mL), dried over Na₂SO₄, filtered and concentrated in vacuum. The crude product was purified by flash chromatography on silica gel column using 0-1% MeOH/DCM as eluent, to provide the title product 340-c as a white solid (35 g, 58.8%).

### Step 4. tert-butyl 3-iodo-6,7-dihydro-5H-pyrazolo[1,5-a]pyrimidine-4-carboxylate (340-d)

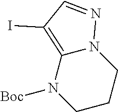 [SMILES: CC(C)(C)OC(=O)N1CCCn2ncc(I)c21]

To a solution of 340-c (38 g, 0.170 mol) in CH₃CN (500 mL) was added NIS (45.95 g, 0.204 mol) at 0° C. The reaction was stirred at 20° C. for 16 hr. The reaction mixture was poured into water (200 mL) and extracted with EtOAc (100 mL×2). The organic layer was washed with water (100 mL), brine (100 mL), dried over Na₂SO₄ and concentrated under reduced pressure. The crude product was purified by flash chromatography on silica gel column using 0-20% EtOAc/petroleum ether as eluent, to provide the title product 340-d as a yellow solid (54 g, 90.9%).

### Step 5. tert-butyl 3-(1-(4-methoxybenzyl)-4-nitro-1H-pyrazol-5-yl)-6,7-dihydropyrazolo[1,5-a]pyrimidine-4(5H)-carboxylate (340-e)

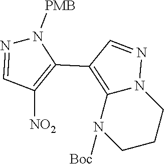

Into dry dioxane (100 mL) was 340-d (5.0 g, 14.3 mmol), 340-a (3.34 g, 14.3 mmol), cesium; 2,2-dimethylpropanoate (3.69 g, 15.8 mmol) and Cs₂CO₃ (7.0 g, 21.5 mmol) were added. The reaction mixture was degassed with N₂ under sonication for 5 minutes, followed by the addition of XPhos (2.05 g, 4.30 mmol) and Pd(OAc)₂ (321.5 mg, 1.43 mmol). The mixture was then degassed again and stirred for 16 hr at 100° C. under N₂ atmosphere. The mixture was filtered over celite and washed with EtOAc (40 mL×2). The filtrate was concentrated under reduced pressure. The crude product was purified by flash chromatography on silica gel column using 20-50% EtOAc/petroleum ether as eluent, to provide the title product 340-e as a brown oil (2.7 g, 33.2% yield, 80% purity). MS (ESI) [M+H]⁺ m/z 455.2.

### Step 6. tert-butyl 3-[4-amino-2-[(4-methoxyphenyl)methyl]pyrazol-3-yl]-6,7-dihydro-5H-pyrazolo[1,5-a]pyrimidine-4-carboxylate (340-f)

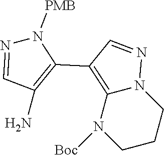

340-e (2.70 g, 4.75 mmol, 80% purity) was dissolved in the mixture of H₂O (9 mL) and EtOH (27 mL), then NH₄Cl (2.54 g, 47.5 mmol), Zn (3.11 g, 47.5 mmol) and 2N HCl/H₂O (0.24 mL, 0.480 mmol) were added. The reaction mixture was heated at 50° C. for 1 hour. The mixture was filtered over celite and washed with MeOH (20 mL×2). After concentration under reduced pressure, the mixture was diluted with water (20 mL) and extracted with DCM (20 mL×2). The combined organic layers were washed with brine (20 mL), dried over Na₂SO₄, filtered and concentrated under reduced pressure. The crude product was purified by flash chromatography on silica gel column using 25-100% EtOAc/petroleum ether as eluent, to provide the title product 340-f as a brown oil (1.4 g, 69.4% yield). MS (ESI) [M+H]⁺ m/z 425.2.

### Step 7. 1-(4-methoxybenzyl)-5-(4,5,6,7-tetrahydropyrazolo[1,5-a]pyrimidin-3-yl)-1H-pyrazol-4-amine (340-g)

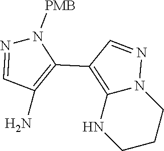

The solution of 340-f (1.40 g, 3.30 mmol) in 4N HCl/MeOH (20.6 mL, 82.4 mmol) was stirred at 20° C. for 2 hr. The reaction was concentrated to provide the title product 340-g as a yellow solid (1.20 g, 91.6%). MS (ESI) [M+H]⁺ m/z 325.2.

### Step 8. 6-(2-fluoro-6-methylphenyl)-10-(4-methoxybenzyl)-4,5-dihydro-3H,10H-2,2a,5a,7,9,10-hexaazabenzo[cd]cyclopenta[h]azulene (340-h)

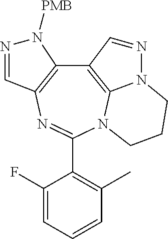

340-g (300 mg, 0.755 mmol, 2HCl) was dissolved in t-BuOH (15 mL). Some drops of THF, K₂CO₃ (521.8 mg, 3.78 mmol) and 2-fluoro-6-methyl-benzaldehyde (104.3 mg, 0.755 mmol) were added. After stirring for 30 minutes at 20° C., I₂ (575 mg, 2.27 mmol) was added in one portion and reaction mixture was stirred at 20° C. for 1 hour. The reaction mixture was quenched with Na₂S₂O₃ aqueous solution (20 mL, 5 wt %) and stirred at 20° C. for 30 minutes. The reaction mixture was concentrated under reduced pressure to remove the organic solvents and the rest aqueous layer was extracted with DCM (20 mL×2). The combined organic layers were washed with brine (20 mL), dried over Na₂SO₄, filtered and concentrated under reduced pressure. The crude product was purified by flash chromatography on silica gel column using 25-100% EtOAc/petroleum ether as eluent, to provide the title product 340-h as a yellow oil (154 mg, 46.1%). MS (ESI) [M+H]⁺ m/z 443.2.

### Step 9. 9-(2-fluoro-6-methylphenyl)-3,6,7,8-tetrahydro-2,3,5,5a,8a,10 hexaazabenzo[cd]cyclopenta[h]azulene (Compound 340)

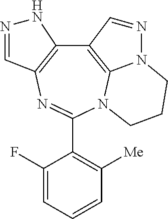 [SMILES: Cc1cccc(F)c1C1=Nc2cn[nH]c2-c2cnn3c2N1CCC3]

A mixture of 340-h (200 mg, 0.452 mmol) in TFA (6 mL) was stirred for 16 hr at 20° C. The reaction mixture was concentrated under reduced pressure. The crude product was purified by reverse phase preparative HPLC (column: Phenomenex Gemini-NX 150×30 mm×5 μm; mobile phase: [water (0.225% FA)-MeCN]; B %: 6%-46%, 9 min), to provide the title product 340 as a yellow solid (55.3 mg, 38.0%). MS (ESI) [M+H]⁺ m/z 323.1.

### Example 5. 9-(2-chloro-6-fluorophenyl)-3,6,7,8-tetrahydro-2,3,5,5a,8a,10-hexaazabenzo[cd]cyclopenta[h]azulene (Compound 341)

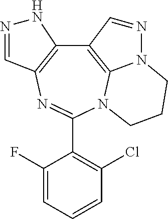 [SMILES: Fc1cccc(Cl)c1C1=Nc2cn[nH]c2-c2cnn3c2N1CCC3]

The title compound was made from diamine intermediate 340-g (Example 4, Step 7), following synthetic procedure similar as described in the synthesis of compound 340 (Example 4, Steps 8-9), except that 2-chloro-6-fluorobenzaldehyde was used in the place of 2-fluoro-6-methylbenzaldehyde. The crude product was purified by reverse phase preparative HPLC (column: Phenomenex Gemini-NX 150×30 mm×5 um; mobile phase: [water (0.225% FA)-MeCN]; B %: 13%-53%, 9 min), to provide the title product 341 as a yellow solid (20 mg, 14.1%). MS (ESI) [M+H]⁺ m/z 343.0.

### Example 6. 9-(2,6-dichlorophenyl)-3,6,7,8-tetrahydro-2,3,5,5a,8a,10-hexaazabenzo[cd]cyclopenta[h]azulene (Compound 342)

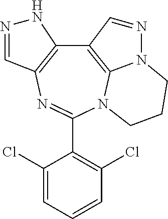 [SMILES: Clc1cccc(Cl)c1C1=Nc2cn[nH]c2-c2cnn3c2N1CCC3]

The title compound was made from diamine intermediate 340-g (Example 4, Step 7), following synthetic procedure similar as described in the synthesis of compound 340 (Example 4, Steps 8-9), except that 2,6-dichlorobenzaldehyde was used in the place of 2-fluoro-6-methylbenzaldehyde. The crude product was purified by reverse phase preparative HPLC (column: Phenomenex Gemini-NX 150×30 mm×5 μm; mobile phase: [water (0.225% FA)-MeCN]; B %: 6%-46%, 9 min), to provide the title product 342 as a yellow solid (44.8 mg, 24.9%). MS (ESI) [M+H]⁺ m/z 359.2.

### Example 7. 9-(2,6-difluoro-4-methylphenyl)-3,6,7,8-tetrahydro-2,3,5,5a,8a,10 hexaazabenzo[cd]cyclopenta[h]azulene (Compound 343)

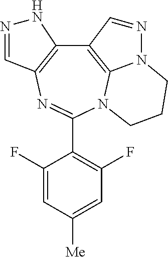 [SMILES: Cc1cc(F)c(C2=Nc3cn[nH]c3-c3cnn4c3N2CCC4)c(F)c1]

The title compound was made from intermediate 340-g (Example 4, Step 7), following synthetic procedure similar as described in the synthesis of compound 340 (Example 4, Steps 8-9), except that 2,6-difluoro-4-methyl-benzaldehyde was used in the place of 2-fluoro-6-methylbenzaldehyde. The crude product was purified by reverse phase preparative HPLC (column: Phenomenex Gemini-NX 150×30 mm×5 μm; mobile phase: [water (0.225% FA)-MeCN]; B %: 13%-53%, 9 min), to provide the title product 343 as a yellow solid (17.4 mg, 9.42%). MS (ESI) [M+H]⁺ m/z 341.1.

### Example 8. 10-(2,6-difluorophenyl)-6,7,8,9-tetrahydro-3H-2,3,5,5a,9a,11-hexaazadicyclopenta[b,kl]heptalene (Compound 344)

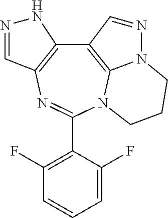 [SMILES: Fc1cccc(F)c1C1=Nc2cn[nH]c2-c2cnn3c2N1CCC3]

### Step 1. tert-butyl 2-allylhydrazine-1-carboxylate (344-a)

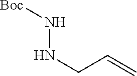

The mixture of tert-butyl N-aminocarbamate (10 g, 75.6 mmol), 3-bromoprop-1-ene (9.15 g, 75.6 mmol) and K₂CO₃ (20.9 g, 151 mmol) in THE (100 mL) was stirred at 80° C. for 16 hr. The mixture was filtered, and the filtrate was partitioned between EtOAc (200 mL) and brine (200 mL). The organic layer was washed with brine (200 mL), dried over anhydrous Na₂SO₄ and concentrated in vacuum. The crude product was purified by flash chromatography on silica gel column using 10-30% EtOAc/petroleum ether as eluent, to provide the title product 344-a as a colorless oil (8 g, 61.4%).

### Step 2. 3-allylhydrazine dihydrochloride (344-b)

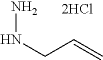

The mixture of 344-a (8 g, 46.4 mmol) in HCl/EtOAc (4 M, 69.6 mL) and MeOH (50 mL) was stirred at 25° C. for 3 hr. The mixture was concentrated in vacuum to provide the title product 344-b as a white solid (6.74 g, crude, 2HCl). It was directly used in the next step without further purification.

### Step 3. ethyl 1-allyl-5-amino-1H-pyrazole-4-carboxylate (344-c)

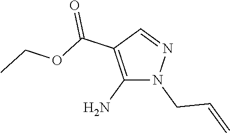

The mixture of 344-b (6.7 g, 46.2 mmol, 2 HCl), ethyl 2-cyano-3-ethoxy-prop-2-enoate (7.82 g, 46.2 mmol) and TEA (14 g, 138 mmol) in i-PrOH (100 mL) was stirred at 100° C. for 5 hr. The mixture was partitioned between EtOAc (30 mL) and brine (30 mL). The aqueous was extracted with EtOAc (30 mL×3). The combined organic layers were washed with brine (30 mL), dried over anhydrous Na₂SO₄, filtered and concentrated in vacuum to afford the crude product. The crude product was purified by flash chromatography on silica gel column using 0-40% EtOAc/petroleum ether as eluent, to provide the title product 344-c as a yellow oil (6.2 g, 68.0%). MS (ESI) [M+H]⁺ m/z 195.1.

### Step 4. ethyl 1-allyl-5-(bis(tert-butoxycarbonyl)amino)-1H-pyrazole-4-carboxylate (344-d)

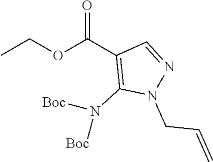 [SMILES: C=CCn1ncc(C(=O)OCC)c1N(C(=O)OC(C)(C)C)C(=O)OC(C)(C)C]

A mixture of 344-c (2 g, 10.2 mmol), Boc₂O (4.92 g, 22.5 mmol) and DMAP (125 mg, 1.02 mmol) in THF (30 mL) was stirred at 60° C. for 3 hr. The mixture was partitioned between EtOAc (100 mL) and brine (100 mL). The organic layer was washed with brine (50 mL), dried over anhydrous Na₂SO₄, filtered and concentrated in vacuum to afford the crude product. The crude product was purified by flash chromatography on silica gel column using 5-30% EtOAc/petroleum ether as eluent, to provide the title product 344-d as a yellow solid (3.1 g, 76.5%).

### Step 5. ethyl 1-allyl-5-((tert-butoxycarbonyl)amino)-1H-pyrazole-4-carboxylate (344-e)

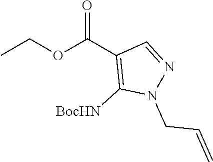 [SMILES: C=CCn1ncc(C(=O)OCC)c1NC(=O)OC(C)(C)C]

A mixture of 344-d (3 g, 7.59 mmol) and TFA (1.73 g, 15.1 mmol) in DCM (20 mL) was stirred at 20° C. for 3 hr. The mixture was concentrated in vacuum to provide the title product 334-e as a yellow oil (2.24 g, crude). It was used into next step without further purification.

### Step 6. ethyl 1-allyl-5-(allyl(tert-butoxycarbonyl)amino)-1H-pyrazole-4-carboxylate (344-f)

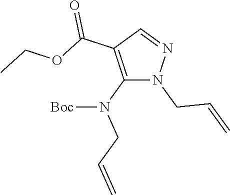 [SMILES: C=CCN(C(=O)OC(C)(C)C)c1c(C(=O)OCC)cnn1CC=C]

A mixture of 344-e (2.24 g, 6.83 mmol), 3-bromoprop-1-ene (3.30 g, 27.3 mmol) and K₂CO₃ (1.89 g, 13.6 mmol) in THF (50 mL) was stirred at 70° C. for 16 hr. The mixture was partitioned between EtOAc (50 mL) and brine (50 mL). The aqueous layer was extracted with EtOAc (50 mL×2). The combined organic layers were washed with brine (50 mL), dried over anhydrous Na₂SO₄, filtered and concentrated in vacuum to afford the crude product. The crude product was purified by flash chromatography on silica gel column using 5-20% EtOAc/petroleum ether as eluent, to provide the title product 334-f as a yellow oil (2.1 g, 82.5%).

### Step 7. 4-(tert-butyl) 3-ethyl 5,8-dihydro-4H-pyrazolo[1,5-a][1,3]diazepine-3,4-dicarboxylate (344-g)

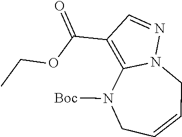 [SMILES: CCOC(=O)c1cnn2c1N(C(=O)OC(C)(C)C)CC=CC2]

A mixture of 344-f (2 g, 5.96 mmol) and [1,3-bis(2,4,6-trimethylphenyl)imidazolidin-2-ylidene]-dichloro-[(2-isopropoxyphenyl)methylene]ruthenium (186 mg, 298 μmol) in toluene (50 mL) was stirred at 80° C. for 1 hour. The mixture was concentrated in vacuum to afford the crude product. The crude product was purified by flash chromatography on silica gel column using 5-20% EtOAc/petroleum ether as eluent, to provide the title product 344-g as a yellow solid (1.2 g, 65.4% yield). MS (ESI) [M+H]⁺ m/z 307.2.

### Step 8. 4-(tert-butyl) 3-ethyl 5,6,7,8-tetrahydro-4H-pyrazolo[1,5-a][1,3]diazepine-3,4-dicarboxylate (344-h)

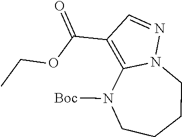 [SMILES: CCOC(=O)c1cnn2c1N(C(=O)OC(C)(C)C)CCCC2]

To a solution of 344-g (1.2 g, 3.90 mmol) in THE (30 mL) was added Pd/C (415 mg, 0.390 mmol, 10 wt % Pd with 50 wt % water) under nitrogen. The suspension was degassed under vacuum and purged with H₂ several times. The mixture was stirred under H₂ (15 psi) at 20° C. for 2 hr. The mixture was filtered and the filtrate was concentrated in vacuum to provide the title product 344-h as a white solid (1.21 g, 100%). The crude product was used directly in the next step without further purification. MS (ESI) [M+H]⁺ m/z 309.2.

### Step 9. 5,6,7,8-tetrahydro-4H-pyrazolo[1,5-a][1,3]diazepine (344-i)

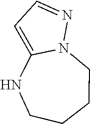

A mixture of 344-h (0.05 g, 161 μmol) in HCl (1.59 g, 16.1 mmol, 12M) was stirred at 100° C. for 16 hr. The mixture was concentrated in vacuum to provide the title product 344-i as a yellow oil (0.42 g, crude, HCl). It was used directly in the next step without further purification.

### Step 10. tert-butyl 5,6,7,8-tetrahydro-4H-pyrazolo[1,5-a][1,3]diazepine-4-carboxylate (344-j)

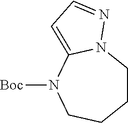 [SMILES: CC(C)(C)OC(=O)N1CCCCn2nccc21]

A mixture of 344-i (0.332 g, 2.42 mmol), DMAP (887 mg, 7.2 mmol), TEA (1.22 g, 12.1 mmol) and Boc₂O (1.58 g, 7.26 mmol) in THE (30 mL) was stirred at 80° C. for 48 hr. The mixture was partitioned between EtOAc (30 mL) and brine (30 mL). The aqueous was extracted with EtOAc (30 mL×2). The combined organic layers were washed with brine (30 mL), dried over anhydrous Na₂SO₄, filtered and concentrated in vacuum to afford the crude product. The crude product was purified by flash chromatography on silica gel column using 10-50% EtOAc/petroleum ether as eluent, to provide the title product 344-j as a yellow oil (0.16 g, 26.4%).

### Step 11. tert-butyl 3-iodo-5,6,7,8-tetrahydro-4H-pyrazolo[1,5-a][1,3]diazepine-4-carboxylate (344-k)

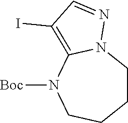 [SMILES: CC(C)(C)OC(=O)N1CCCCn2ncc(I)c21]

A mixture of 344-j (0.1 g, 0.421 mmol) and NIS (94.8 mg, 421 μmol) in MeCN (2 mL) was stirred at 20° C. for 16 hr. The mixture was partitioned between EtOAc (10 mL) and brine (10 mL). The organic layer was washed with brine (5 mL), dried over anhydrous Na₂SO₄, filtered and concentrated in vacuum to afford the crude product. The crude product was purified by flash chromatography on silica gel column using 10-40% EtOAc/petroleum ether as eluent, to provide the title product 344-k as a white solid (0.11 g, 71.8% yield).

### Step 12. tert-butyl 3-(1-(4-methoxybenzyl)-4-nitro-1H-pyrazol-5-yl)-5,6,7,8-tetrahydro-4H-pyrazolo[1,5-a][1,3]diazepine-4-carboxylate (344-l)

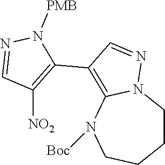

A mixture of 344-k (0.11 g, 302 μmol), 1-[(4-methoxyphenyl)methyl]-4-nitro-pyrazole (70.6 mg, 302 μmol), Pd(OAc)₂ (6.80 mg, 30.2 μmol), CuI (5.77 mg, 30.2 μmol), XPhos (43.3 mg, 90.8 μmol), Cs₂CO₃ (148 mg, 454 μmol) and cesium-2,2-dimethylpropanoate (77.9 mg, 333 μmol) in dioxane (5 mL) was degassed and purged with N₂ for 3 times, and then the mixture was stirred at 110° C. for 16 hr under N₂ atmosphere. The mixture was partitioned between EtOAc (20 mL) and brine (20 mL). The organic layer was washed with brine (10 mL), dried over anhydrous Na₂SO₄, filtered and concentrated in vacuum to afford the crude product. The crude product was purified by flash chromatography on silica gel column using 10-40% EtOAc/petroleum ether as eluent, to provide the title product 344-l as a white solid (0.1 g, 70.4%).

### Step 13. tert-butyl 3-(4-amino-1-(4-methoxybenzyl)-1H-pyrazol-5-yl)-5,6,7,8-tetrahydro-4H-pyrazolo[1,5-a][1,3]diazepine-4-carboxylate (344-m)

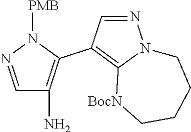

To a solution of 344-l (0.1 g, 0.213 mmol) in THE (3 mL) was added Pd/C (68.1 mg, 10 wt % Pd with 50 wt % water) under N₂. The suspension was degassed under vacuum and purged with H₂ several times. The mixture was stirred under H₂ (15 psi) at 30° C. for 2 hr. The mixture was filtered and the filtrate was concentrated in vacuum to provide the title product 344-m as a yellow oil (0.093 g, crude). It was used directly in the next step without further purification. MS (ESI) [M+H]⁺ m/z 439.2.

### Step 14. 1-(4-methoxybenzyl)-5-(5,6,7,8-tetrahydro-4H-pyrazolo[1,5-a][1,3]diazepin-3-yl)-1H-pyrazol-4-amine (344-n)

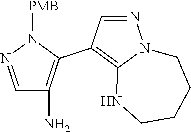

A mixture of 344-m (0.093 g, 0.212 mmol) in HCl/EtOAc (4 M, 1.06 mL) was stirred at 30° C. for 1 hour. The mixture was concentrated in vacuum to provide the title product 344-n as a yellow solid (0.079 g, crude, HCl). It was used directly in the next step without further purification.

### Step 15. 10-(2,6-difluorophenyl)-6,7,8,9-tetrahydro-3H-2,3,5,5a,9a,11-hexaazadicyclopenta [b,kl]heptalene (Compound 344)

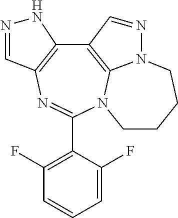 [SMILES: Fc1cccc(F)c1C1=Nc2cn[nH]c2-c2cnn3c2N1CCCC3]

The title compound was made from intermediate 344-n (Example 8, Step 14), following synthetic procedure similar as described in the synthesis of compound 340 (Example 4, Steps 8-9), except that 344-n was used in the place of 340-g and 2,6-difluorobenzaldehyde was used in the place of 2-fluoro-6-methyl-benzaldehyde. The crude product was purified by reverse phase preparative HPLC (column: Phenomenex Gemini-NX 80×40 mm×3 μm; mobile phase: [water (10 mM NH₄HCO₃)-MeCN]; B %: 22%-52%, 7.8 min), to provide the title product 344 as a yellow solid (0.007 g, 17.0%). MS (ESI) [M+H]⁻ m/z 341.1.

### Example 8. 9-(2,6-difluorophenyl)-6-methyl-3,6,7,8-tetrahydro-2,3,5,5a,8a,10-hexaazabenzo[cd] cyclopenta[h]azulene (Compound 345)

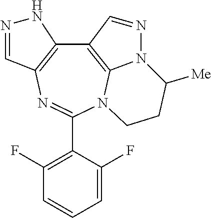 [SMILES: CC1CCN2C(c3c(F)cccc3F)=Nc3cn[nH]c3-c3cnn1c32]

### Step 1. 7-methyl-4,5,6,7-tetrahydropyrazolo[1,5-a]pyrimidine (345-a)

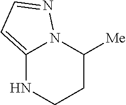

To a solution of 1H-pyrazol-5-amine (10 g, 0.120 mol) in dioxane (250 mL) was added TEA (36.5 g, 0.361 mol) and 1,3-dibromobutane (25.99 g, 0.120 mol). The mixture was stirred at 100° C. for 16 hr. The reaction mixture was added H₂O (200 mL) and extracted with EtOAc (200 mL×3). The combined organic layers were washed with brine (200 mL), dried with anhydrous Na₂SO₄, filtered and concentrated under reduced pressure to give a residue. The crude product was purified by flash chromatography on silica gel column using 0-100% EtOAc/petroleum ether as eluent, to provide the title product 345-a as a white solid (3.8 g, 22.4% yield). MS (ESI) [M+H]⁺ m/z 138.1.

### Step 2. 3-iodo-7-methyl-4,5,6,7-tetrahydropyrazolo[1,5-a]pyrimidine (345-b)

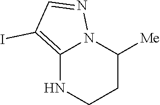 [SMILES: CC1CCNc2c(I)cnn21]

To a solution of 345-a (2.3 g, 16.7 mmol) in DCM (40 mL) was added NIS (3.77 g, 16.7 mmol). The mixture was stirred at 25° C. for 2 hr. The reaction mixture was added H₂O (10 mL) and extracted with EtOAc (10 mL×3). The combined organic layers were washed with brine (20 mL), dried with anhydrous Na₂SO₄, filtered and concentrated under reduced pressure to give a residue. The crude product was purified by flash chromatography on silica gel column using 0-50% EtOAc/petroleum ether as eluent, to provide the title product 345-b as a yellow solid (3.3 g, 74.8% yield).

### Step 3. tert-butyl 3-iodo-7-methyl-6,7-dihydropyrazolo[1,5-a]pyrimidine-4(5H)-carboxylate (345-c)

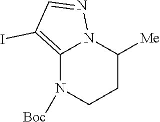 [SMILES: CC1CCN(C(=O)OC(C)(C)C)c2c(I)cnn21]

To a solution of 345-b (1.5 g, 5.70 mmol) in THE (15 mL) was added tert-butoxycarbonyl tert-butyl carbonate (1.87 g, 8.55 mmol), DMAP (69.66 mg, 0.570 mmol). The mixture was stirred at 60° C. for 16 hr. The reaction mixture was added H₂O (10 mL) and extracted with EtOAc (10 mL×3). The combined organic layers were washed with brine (20 mL), dried with anhydrous Na₂SO₄, filtered and concentrated under reduced pressure to give a residue. The crude product was purified by flash chromatography on silica gel column using 0-15% EtOAc/petroleum ether as eluent, to provide the tide product 345-c as a colorless oil (1.47 g, 70.5% yield). LCMS (ESI) [M+H]⁺ m/z: 264.0.

### Step 4. tert-butyl 3-(1-(4-methoxybenzyl)-4-nitro-1H-pyrazol-5-yl)-7-methyl-6,7-dihydropyrazolo[1,5-a]pyrimidine-4(5H)-carboxylate (345-d)

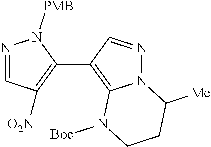

A mixture of 345-c (1.47 g, 4.05 mmol), 1-[(4-methoxyphenyl)methyl]-4-nitro-pyrazole (943 mg, 4.05 mmol), cesium; 2,2-dimethylpropanoate (1.04 g, 4.45 mmol), Cs₂CO₃ (1.98 g, 6.07 mmol), CuI (77.0 mg, 0.404 mmol), XPhos (578 mg, 1.21 mmol) and Pd(OAc)₂ (90.8 mg, 0.404 mmol) in dioxane (15 mL) was degassed and purged with N₂ for 3 times, and then the mixture was stirred at 110° C. for 24 hr under N₂ atmosphere. The reaction mixture was treated with H₂O (10 mL) and extracted with EtOAc (20 mL×3). The combined organic layers were washed with brine (20 mL), dried with anhydrous Na₂SO₄, filtered and concentrated under reduced pressure to give a residue. The crude product was purified by flash chromatography on silica gel column using 0-30% EtOAc/petroleum ether as eluent, to provide the title product 345-d as a yellow oil (900 mg, 46.9% yield). LCMS (ESI) [M+H]⁺ m/z: 469.2.

### Step 5. tert-butyl 3-(4-amino-1-(4-methoxybenzyl)-1H-pyrazol-5-yl)-7-methyl-6,7-dihydropyrazolo[1,5-a]pyrimidine-4(5H)-carboxylate (345-e)

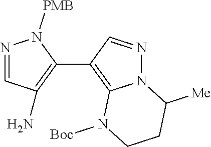

Into a solution of 345-d (300 mg, 0.640 mmol) in THF (3 mL) was added Pd/C (681 mg, 10 wt % Pd with 50 wt % water). The mixture was degassed and backfilled with hydrogen gas thrice and allowed to stir at 25° C. for 16 hr under hydrogen (15 psi). The reaction mixture was filtered through the celite and the filtrate was concentrated in vacuum to provide the title product 345-e as a yellow oil (260 mg, crude). LCMS (ESI) [M+H]⁺ m/z: 439.2.

### Step 6. 1-(4-methoxybenzyl)-5-(7-methyl-4,5,6,7-tetrahydropyrazolo[1,5-a]pyrimidin-3-yl)-1H-pyrazol-4-amine (345-f)

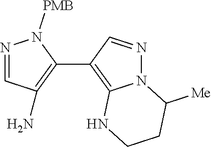

A solution of 345-e (260 mg, 0.592 mmol) in HCl/MeOH (4 M, 5 mL) was stirred at 25° C. for 1 hr. The reaction mixture was concentrated in vacuum to provide the title product 345-f as a yellow oil (200 mg, crude). LCMS (ESI) [M+H]⁺ m/z: 339.1.

### Step 7. 9-(2,6-difluorophenyl)-6-methyl-3,6,7,8-tetrahydro-2,3,5,5a,8a,10-hexaazabenzo[cd]cyclopenta[h]azulene (Compound 345)

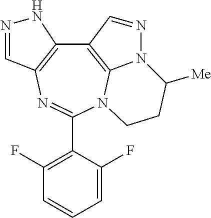 [SMILES: CC1CCN2C(c3c(F)cccc3F)=Nc3cn[nH]c3-c3cnn1c32]

The title compound was made from intermediate 345-f (Example 72, Step 6), following synthetic procedure similar as described in the synthesis of compound 340 (Example 4, Steps 8-9), except that 345-f was used in the place of 340-g, and that, 2,6-difluorobenzaldehyde was used in the place of 2-fluoro-6-methyl-benzaldehyde. The crude product was purified by reverse phase preparative HPLC (column: Welch Xtimate C18 150×25 mm×5 μm; mobile phase: [water (0.225% FA)-MECN]; B %: 25%-55%, 7.8 min), to provide the title compound 345 as a yellow solid (24.6 mg, 11.7%). MS (ESI) [M+H]⁺ m/z 341.1.

### Example 9. (R)-9-(2,6-difluorophenyl)-6-methyl-3,6,7,8-tetrahydro-2,3,5,5a,8a,10-hexaazabenzo [cd]cyclopenta[h]azulene (Compound 346)

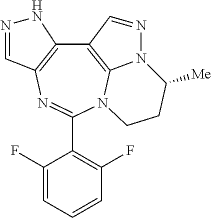 [SMILES: C[C@@H]1CCN2C(c3c(F)cccc3F)=Nc3cn[nH]c3-c3cnn1c32]

Compound 345 (13.0 mg) was separated by chiral SFC (column: Daicel Chiralpak AD (250 mm×30 mm×10 μm); mobile phase: [0.1% NH₃H₂O IPA]; B %: 40%-40%) to provide the title product 346 as a yellow solid (5.47 mg, 41.8%). The absolute configuration is arbitrarily assigned. MS (ESI) [M+H]⁺ m/z 341.1.

### Example 10. (S)-9-(2,6-difluorophenyl)-6-methyl-3,6,7,8-tetrahydro-2,3,5,5a,8a,10-hexaazabenzo[cd] cyclopenta[h]azulene (Compound 347)

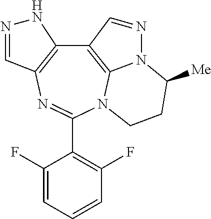 [SMILES: C[C@H]1CCN2C(c3c(F)cccc3F)=Nc3cn[nH]c3-c3cnn1c32]

Compound 345 (13.0 mg) was purified by SFC (column: DAICEL CHIRALPAK AD (250 mm×30 mm×10 μm); mobile phase: [0.1% NH₃H₂O IPA]; B %: 40%-40%, min) to provide the title product 347 as a yellow solid (4.74 mg, 36.2%). The absolute configuration is arbitrarily assigned. MS (ESI) [M+H]⁺ m/z 341.1.

### Example 11. 9-(2,6-difluorophenyl)-7-methyl-2,6,7,8-tetrahydro-2,3,5,5a,8a,10-hexaazabenzo[cd] cyclopenta[h]azulene (Compound 348)

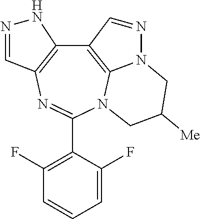 [SMILES: CC1CN2C(c3c(F)cccc3F)=Nc3cn[nH]c3-c3cnn(c32)C1]

### Step 1. 6-methyl-4,5,6,7-tetrahydropyrazolo[1,5-a]pyrimidine (348-a)

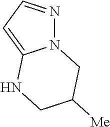

To a solution of 1H-pyrazol-5-amine (1.54 g, 18.5 mmol) and 1,3-dibromo-2-methyl-propane (4 g, 18.5 mmol) in dioxane (1 mL) was added TEA (5.62 g, 55.6 mmol). The mixture was stirred at 100° C. for 16 hr. The reaction mixture was concentrated under reduced pressure to remove dioxane. The crude product was purified by flash chromatography on silica gel column using 0-100% EtOAc/petroleum ether as eluent, to provide the title product 348-a as a yellow solid (3.8 g, 22.4% yield).

### Step 2. tert-butyl 6-methyl-6,7-dihydropyrazolo[1,5-a]pyrimidine-4(5H)-carboxylate (348-b)

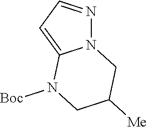 [SMILES: CC1CN(C(=O)OC(C)(C)C)c2ccnn2C1]

To a solution of 348-a (1.1 g, 8.02 mmol) in THE (12 mL) was added Boc₂O (2.63 g, 12.0 mmol) and DMAP (1.96 g, 16.0 mmol). The mixture was stirred at 25° C. for 3 hr. The reaction mixture was diluted with H₂O (30 mL) and extracted with EtOAc (30 mL×3). The combined organic layers were washed with brine (30 mL×2), dried with anhydrous Na₂SO₄, filtered and concentrated under reduced pressure to give a residue. The crude product was purified by flash chromatography on silica gel column using 0-15% EtOAc/petroleum ether as eluent, to provide the title product 348-b as a colorless oil (700 mg, 34.9% yield).

### Step 3. tert-butyl 3-iodo-6-methyl-6,7-dihydropyrazolo[1,5-a]pyrimidine-4(5H)-carboxylate (348-c)

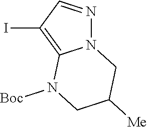 [SMILES: CC1CN(C(=O)OC(C)(C)C)c2c(I)cnn2C1]

To a solution of 348-b (700 mg, 2.95 mmol) in DCM (1 mL) was added NIS (663 mg, 2.95 mmol). The mixture was stirred at 25° C. for 1 hour. The reaction mixture was concentrated under reduced pressure to remove DCM. The crude product was purified by flash chromatography on silica gel column using 0-40% EtOAc/petroleum ether as eluent, to provide the title product 348-c as a yellow oil (1.07 g, 100% yield).

### Step 4. tert-butyl 3-(1-(4-methoxybenzyl)-4-nitro-1H-pyrazol-5-yl)-6-methyl-6,7-dihydropyrazolo[1,5-a]pyrimidine-4(5H)-carboxylate (348-d)

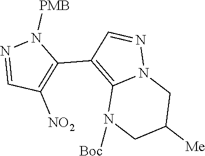

A flask charged with 340-a (128 mg, 0.55 mmol), 348-c (200 mg, 0.55 mmol), Cs₂CO₃ (269.1 mg, 0.83 mmol) and cesium; 2,2-dimethylpropanoate (167 mg, 0.72 mmol) in 25 mL dry dioxane (10 mL) was degassed with N₂ under sonication, followed by the addition of dicyclohexyl-[2-(2,4,6-triisopropylphenyl)phenyl] phosphane (78.7 mg, 0.16 mmol), Pd(OAc)₂ (12.4 mg, 0.06 mmol) and CuI (10.5 mg, 0.06 mmol). The mixture was then degassed again and stirred 16 hr at 110° C. The reaction mixture was diluted with H₂O (30 mL) and extracted with EtOAc (30 mL×3). The combined organic layers were washed with brine (30 mL×2), dried with anhydrous Na₂SO₄, filtered and concentrated under reduced pressure to give a residue. T The crude product was purified by flash chromatography on silica gel column using 0-30% EtOAc/petroleum ether as eluent, to provide the title product 348-d as a yellow oil (100 mg, 36.0% yield). LCMS (ESI) [M+H]⁺ m/z: 469.2.

### Step 5. tert-butyl 3-(4-amino-1-(4-methoxybenzyl)-1H-pyrazol-5-yl)-6-methyl-6,7-dihydropyrazolo[1,5-a]pyrimidine-4(5H)-carboxylate (348-e)

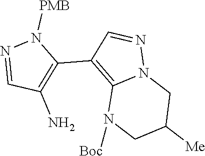

To a solution of 348-d (100 mg, 0.21 mmol) in THE (6 mL) was added Pd/C (227 mg, 10 wt % Pd with 50 wt % water) under N₂ atmosphere. The suspension was degassed and purged with H₂ for 3 times. The mixture was stirred under H₂ (15 Psi or atm.) at 25° C. for 16 hr. The reaction mixture was filtered and concentrated under reduced pressure to provide the title product 348-e as a yellow oil (90 mg, crude).

### Step 6. 1-(4-methoxybenzyl)-5-(6-methyl-4,5,6,7-tetrahydropyrazolo[1,5-a]pyrimidin-3-yl)-1H-pyrazol-4-amine (348-f)

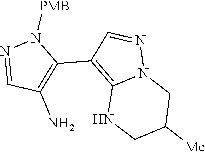

A solution of 348-e (90 mg, 0.21 mmol) in HCl/MeOH (4M, 2 mL) was allowed to stir at 25° C. for 1 hour. The reaction mixture was concentrated in vacuum to provide the title product 348-f as a yellow oil (100 mg, crude).

### Step 7. 9-(2,6-difluorophenyl)-7-methyl-7,8-dihydro-2H,6H-2,3,5,5a,8a,10-hexaazabenzo[cd]cyclopenta[h]azulene (348)

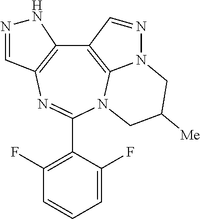 [SMILES: CC1CN2C(c3c(F)cccc3F)=Nc3cn[nH]c3-c3cnn(c32)C1]

The title compound was made from intermediate 348-f (Example 11, Step 6), following synthetic procedure similar as described in the synthesis of compound 340 (Example 4, Steps 8-9), except that 2,6-difluorobenzaldehyde was used in the place of 2-fluoro-6-methyl-benzaldehyde. The crude product was purified by reverse phase preparative HPLC (column: Welch Xtimate C18 150×25 mm×5μm; mobile phase: [water (0.225% FA)-MECN]; B %: 25%-55%, 7.8 min), to provide the title compound 348 as a yellow solid (24.6 mg, 11.7%). MS (ESI) [M+H]⁺ m/z 341.1.

### Example 12. (11R)-8-(2,6-difluorophenyl)-11-methyl-3,4,7,9,13,14-hexazatetracyclo[7.6.1.02,6.013,16] hexadeca-1(16),2(6),4,7,14-pentaene (Compound 349)

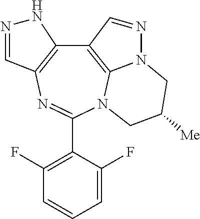 [SMILES: C[C@@H]1CN2C(c3c(F)cccc3F)=Nc3cn[nH]c3-c3cnn(c32)C1]

Compound 348 (16.5 mg) was subjected to separation by chiral SFC (column: Daicel Chiralpak AD (250 mm×30 mm×10 m); mobile phase: [0.1% NH₃H₂O IPA]; B %: 35%-35%) to provide the title product 349 as a yellow solid (8 mg, 49.0%). The absolute configuration is arbitrarily assigned. MS (ESI) [M+H]⁺ m/z 341.1.

### Example 13. (11S)-8-(2,6-difluorophenyl)-11-methyl-3,4,7,9,13,14-hexazatetracyclo[7.6.1.02,6.013,16] hexadeca-1(16),2(6),4,7,14-pentaene (Compound 350)

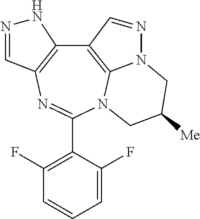 [SMILES: C[C@H]1CN2C(c3c(F)cccc3F)=Nc3cn[nH]c3-c3cnn(c32)C1]

Compound 348 (16.5 mg) was subjected to separation by chiral SFC (column: Daicel Chiralpak AD (250 mm×30 mm×10 μm); mobile phase: [0.1% NH₃H₂O IPA]; B %: 35%-35%, min) to provide the title product 350 as a yellow solid (6 mg, 34.5%). The absolute configuration is arbitrarily assigned. MS (ESI) [M+H]⁺ m/z 341.1.

### Example 14. 9-(2,6-difluorophenyl)-8-methyl-3,6,7,8-tetrahydro-2,3,5,5a,8a,10-hexaazabenzo[cd] cyclopenta[h]azulene (Compound 351)

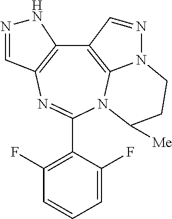 [SMILES: CC1CCn2ncc3c2N1C(c1c(F)cccc1F)=Nc1cn[nH]c1-3]

### Step 1. 5-methyl-4,5,6,7-tetrahydropyrazolo[1,5-a]pyrimidine (351-a)

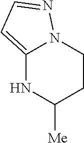

To a solution of 1H-pyrazol-5-amine (14 g, 168 mmol) in dioxane (250 mL) was added TEA (51.1 g, 505 mmol) and 1,3-dibromobutane (36.3 g, 168 mmol). The mixture was stirred at 100° C. for 16 hr. The reaction mixture was added H₂O (200 mL) and extracted with EA (200 mL×3). The combined organic layers were washed with brine (200 mL), dried with anhydrous Na₂SO₄, filtered and concentrated under reduced pressure to give a residue. The crude product was purified by flash chromatography on silica gel column using 0-100% EtOAc/petroleum ether as eluent, to provide the title product 351-a as a white solid (1.4 g, 8.0% yield). LCMS (ESI) [M+H]⁺ m/z: 138.1.

### Step 2. 3-iodo-5-methyl-4,5,6,7-tetrahydropyrazolo[1,5-a]pyrimidine (351-b)

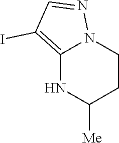 [SMILES: CC1CCn2ncc(I)c2N1]

To a solution of 351-a (1 g, 7.29 mmol) in DCM (15 mL) was added NIS (1.64 g, 7.29 mmol). The mixture was stirred at 25° C. for 2 hr. The reaction mixture was added H₂O (10 mL) and extracted with EtOAc (10 mL×3). The combined organic layers were washed with brine (20 mL), dried with anhydrous Na₂SO₄, filtered and concentrated under reduced pressure to give a residue. The crude product was purified by flash chromatography on silica gel column using 0-50% EtOAc/petroleum ether as eluent, to provide the title product 351-b as a yellow solid (1.75 g, 87.6% yield). LCMS (ESI) [M+H]⁺ m/z: 263.9.

### Step 3. tert-butyl 3-iodo-5-methyl-6,7-dihydropyrazolo[1,5-a]pyrimidine-4(5H)-carboxylate (351-c)

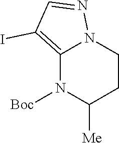 [SMILES: CC1CCn2ncc(I)c2N1C(=O)OC(C)(C)C]

To a solution of 351-b (1 g, 3.80 mmol) in DMF (10 mL) was added Boc₂O (2.49 g, 11.4 mmol), DMAP (1.39 g, 11.4 mmol). The mixture was stirred at 80° C. for 2 hr under microwave. The reaction mixture was concentrated under reduced pressure to give a crude product, which was purified by flash chromatography on silica gel column using 0-15% EtOAc/petroleum ether as eluent, to provide the title product 351-c as a colorless oil (690 mg, 44.9% yield). LCMS (ESI) [M+H]⁺ m/z: 364.0.

### Step 4. tert-butyl 3-(1-(4-methoxybenzyl)-4-nitro-1H-pyrazol-5-yl)-5-methyl-6,7-dihydropyrazolo[1,5-a]pyrimidine-4(5H)-carboxylate (351-d)

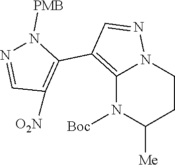

A mixture of 351-c (690 mg, 1.90 mmol), 1-[(4-methoxyphenyl)methyl]-4-nitro-pyrazole (443 mg, 1.90 mmol), cesium-2,2-dimethylpropanoate (489 mg, 2.09 mmol), Cs₂CO₃ (928 mg, 2.85 mmol), CuI (36.1 mg, 0.189 mmol), XPhos (271 mg, 0.569 mmol) and Pd(OAc)₂ (42.6 mg, 0.189 mmol) in dioxane (8 mL) was degassed and purged with N₂ for 3 times, and then the mixture was stirred at 110° C. for 24 hr under N₂ atmosphere. The reaction mixture was filtered and concentrated under reduced pressure to give a residue. The crude product was purified by flash chromatography on silica gel column using 0-50% EtOAc/petroleum ether as eluent, to provide the title product 351-d as a yellow oil (280 mg, 25.8% yield). LCMS (ESI) [M+H]⁺ m/z: 469.2.

### Step 5. tert-butyl 3-(4-amino-1-(4-methoxybenzyl)-1H-pyrazol-5-yl)-5-methyl-6,7-dihydropyrazolo[1,5-a]pyrimidine-4(5H)-carboxylate (351-e)

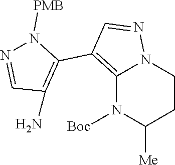

To a solution of 351-d (280 mg, 0.597 mmol) in THE (2 mL) was added Pd/C (636 mg, 10 wt % Pd with 50 wt % water). The mixture was stirred at 25° C. for 16 hr under H₂ (15 psi). The reaction mixture was filtered and concentrated under reduced pressure to provide the title product 351-e as a yellow oil (220 mg, 81.4% yield). LCMS (ESI) [M+H]⁺ m/z: 439.2.

### Step 6. 1-(4-methoxybenzyl)-5-(5-methyl-4,5,6,7-tetrahydropyrazolo[1,5-a]pyrimidin-3-yl)-1H-pyrazol-4-amine (351-f)

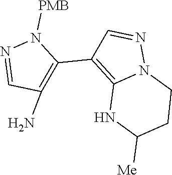

To a solution of 351-e (220 mg, 0.501 mmol) in MeOH (2 mL) was added HCl/MeOH (4M, 2 mL, 8.00 mmol). The mixture was stirred at 25° C. for 16 hr. The reaction mixture was concentrated under reduced pressure to provide the title product 351-f as a yellow oil (169 mg, 99.5% yield). LCMS (ESI) [M+H]⁺ m/z: 339.2.

### Step 7. 9-(2,6-difluorophenyl)-8-methyl-3,6,7,8-tetrahydro-2,3,5,5a,8a,10-hexaazabenzo[cd]cyclopenta[h]azulene (Compound 351)

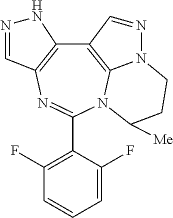 [SMILES: CC1CCn2ncc3c2N1C(c1c(F)cccc1F)=Nc1cn[nH]c1-3]

The title compound was made from intermediate 351-f (Example 14, Step 6), following synthetic procedure similar as described in the synthesis of compound 340 (Example 4, Steps 8-9), except that 2,6-difluorobenzaldehyde was used in the place of 2-fluoro-6-methyl-benzaldehyde. The crude product was purified by flash chromatography (silica gel column using 0-100% EtOAc/petroleum ether as eluent), to provide the title compound 351 as a yellow solid (31.5 mg, 41.9%). MS (ESI) [M+H]⁺ m/z 341.1.

### Example 15. 8-(2,6-difluorophenyl)-3,4,7,9,13,14-hexazatetracyclo[7.6.1.02,6.013,16]hexadeca-1(16),2(6),4,7,14-pentaene (Compound 352)

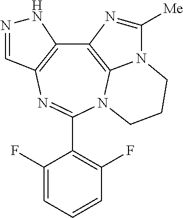 [SMILES: Cc1nc2c3n1CCCN3C(c1c(F)cccc1F)=Nc1cn[nH]c1-2]

### Step 1. tert-butyl (3-(5-iodo-2-methyl-1H-imidazol-1-yl)propyl)carbamate (352-a)

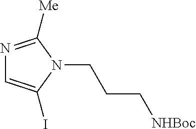 [SMILES: Cc1ncc(I)n1CCCNC(=O)OC(C)(C)C]

Into a 0° C. solution of 5-iodo-2-methyl-1H-imidazole (5.0 g, 24.0 mmol) in THF (50 mL) under nitrogen atmosphere was added NaH (1.06 g, 26.4 mmol, 60 wt % in mineral oil). The mixture was stirred for 1 hour at 0° C. and then tert-butyl N-(3-bromopropyl) carbamate (6.30 g, 26.4 mmol) was added to above solution and warmed to 25° C. and stirred for 16 hr. The mixture was quenched by water (50 mL) and extracted with EtOAc (50 mL×3), organic layers were combined and washed with brine (50 mL), dried over Na₂SO₄ and concentrated in vacuo to give crude product. The crude product was purified by flash chromatography on silica gel column using 0-100% EtOAc/petroleum ether as eluent, to provide the title product 352-a as a yellow solid (3.0 g, 34.2%). MS (ESI) [M+H]⁺ m/z 366.1.

### Step 2. tert-butyl 6-methyl-3,4-dihydroimidazo[1,5-a]pyrimidine-1(2H)-carboxylate (352-b)

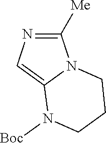 [SMILES: Cc1ncc2n1CCCN2C(=O)OC(C)(C)C]

To a solution of 352-a (7 g, 19.2 mmol) in DMF (140 mL) was added CuI (1.10 g, 5.75 mmol), K₂CO₃ (5.30 g, 38.3 mmol) and N1,N2-dimethylethane-1,2-diamine (1.69 g, 19.2 mmol) at 25° C. under nitrogen atmosphere. The mixture was heated to 100° C. and stirred for 16 hr. The reaction mixture was cooled to 20° C. and filtered through Celite. The filtrate was diluted with water (200 mL) and extracted with EtOAc (100 mL×2). The combined organic phases were washed with brine, dried over Na₂SO₄ and evaporated in vacuo to give a residue. The crude product was purified by flash chromatography on silica gel column using 0-100% EtOAc/petroleum ether as eluent, to provide the title product 352-b as a yellow solid (4.1 g, 90.1%). LCMS (ESI) [M+H]⁻ m/z. 238.2.

### Step 3. 8-(2,6-difluorophenyl)-3,4,7,9,13,14-hexazatetracyclo[7.6.1.02,6.013,16]hexadeca-1(16),2(6),4,7,14-pentaene (Compound 352)

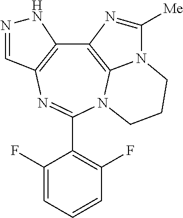 [SMILES: Cc1nc2c3n1CCCN3C(c1c(F)cccc1F)=Nc1cn[nH]c1-2]

The title compound was made from intermediate 352-b (Example 15, Step 3), following synthetic procedure similar as described in the synthesis of compound 340 (Example 4, Steps 4-9), except that 352-b was used in the place of 340-c, and that, 2,6-difluorobenzaldehyde was used in the place of 2-fluoro-6-methyl-benzaldehyde. The crude product was purified by reverse phase preparative HPLC (Phenomenex Gemini-NX 150×30 mm×5 μm; mobile phase: [water (0.05% ammonia hydroxide v/v)-MeCN]; B %: 20%-40%, 12 min), to provide the title compound 352 as a yellow solid (24.6 mg, 11.7%). MS (ESI) [M+H]⁺ m/z 341.1.

### Example 16. 9-(3-bromo-2,6-difluorophenyl)-3,6,7,8-tetrahydro-2,3,5,5a,8a,10-hexaazabenzo[cd] cyclopenta[h]azulene (Compound 353)

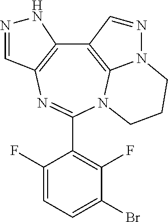 [SMILES: Fc1ccc(Br)c(F)c1C1=Nc2cn[nH]c2-c2cnn3c2N1CCC3]

The title compound was made from intermediate 340-g (Example 4, Step 7), following synthetic procedure similar as described in the synthesis of compound 340 (Example 4, Steps 8-9), except that 3-bromo-2,6-difluorobenzaldehyde was used in the place of 2-fluoro-6-methyl-benzaldehyde. The crude product was purified by reverse phase preparative HPLC (column: Welch Xtimate C18 150×30 mm×5 μm; mobile phase: [water (0.225% FA)-MeCN]; B %: 10%-55%, 7.8 min) to provide the title compound 353 as a yellow solid (28.7 mg, 22.0%). MS (ESI) [M+H]⁺ m/z 407.0.

### Example 17. 9-(2,6-difluoro-3-methylphenyl)-3,6,7,8-tetrahydro-2,3,5,5a,8a,10 Hexaazabenzo[cd]cyclopenta[h]azulene (Compound 354)

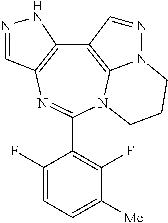 [SMILES: Cc1ccc(F)c(C2=Nc3cn[nH]c3-c3cnn4c3N2CCC4)c1F]

### Step 1. 2,6-difluoro-3-methylbenzaldehyde (354-a)

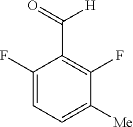 [SMILES: Cc1ccc(F)c(C=O)c1F]

A mixture of 3-bromo-2,6-difluoro-benzaldehyde (1 g, 4.52 mmol), methylboronic acid (325 mg, 5.43 mmol), K₂CO₃ (1.88 g, 13.5 mmol), Pd(dppf)Cl₂ (331 mg, 0.45 mmol) in dioxane (10 mL) was degassed and purged with N₂ for 3 times, and then the mixture was stirred at 100° C. for 16 hr under N₂ atmosphere. The reaction mixture was diluted with H₂O (10 mL) and the mixture was extracted with EtOAC (10 mL×3). The combined organic phase was washed with brine (10 mL×2), dried with anhydrous Na₂SO₄, filtered and concentrated in vacuum. The crude product was purified by flash chromatography on silica gel column using 0-5% EtOAc/petroleum ether as eluent, to provide the title product 354-a as a colorless oil (401 mg, 50.0%).

### Step 2. 9-(2,6-difluoro-3-methylphenyl)-3,6,7,8-tetrahydro-2,3,5,5a,8a,10 Hexaazabenzo[cd]cyclopenta[h]azulene (Compound 354)

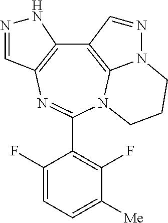 [SMILES: Cc1ccc(F)c(C2=Nc3cn[nH]c3-c3cnn4c3N2CCC4)c1F]

The title compound was made from intermediate 340-g (Example 4, Step 7), following synthetic procedure similar as described in the synthesis of compound 340 (Example 4, Steps 8-9), except that 354-a was used in the place of 2-fluoro-6-methyl-benzaldehyde. The crude product was purified by reverse phase preparative HPLC (column: Welch Xtimate C18 150×30 mm×5 μm; mobile phase: [water (0.225% FA)-MeCN]; B %: 10%-55%, 7.8 min) to provide the title compound 354 as a yellow solid (46.91 mg, 31.5%). MS (ESI) [M+H]⁺ m/z 341.1.

### Example 18. 8-(3-bromo-2-chloro-6-fluoro-phenyl)-3,4,7,9,13,14-hexazatetracyclo [7.6.1.02,6.013,16]hexadeca-1(16),2(6),4,7,14-pentaene (Compound 355)

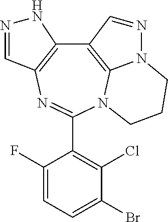 [SMILES: Fc1ccc(Br)c(Cl)c1C1=Nc2cn[nH]c2-c2cnn3c2N1CCC3]

The title compound was made from intermediate 340-g (Example 4, Step 7), following synthetic procedure similar as described in the synthesis of compound 340 (Example 4, Steps 8-9), except that 3-bromo-2-chloro-6-fluorobenzaldehyde was used in the place of 2-fluoro-6-methyl-benzaldehyde. The crude product was purified by reverse phase preparative HPLC (column: YMC-Actus Triart C18 150×30 mm×5 μm; mobile phase: [water (0.225% FA)-MeCN]; B %: 28%-48%, 11 min) to provide the title compound 355 as a yellow solid (16.2 mg, 12.2%). MS (ESI) [M+H]⁺ m/z 423.0.

### Example 19. 9-(2-cyclopropyl-6-fluorophenyl)-3,6,7,8-tetrahydro-2,3,5,5a,8a,10-hexaazabenzo[cd]cyclopenta[h]azulene (Compound 356)

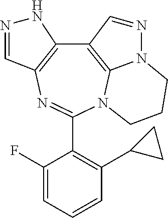 [SMILES: Fc1cccc(C2CC2)c1C1=Nc2cn[nH]c2-c2cnn3c2N1CCC3]

### Step 1. 2-cyclopropyl-6-fluorobenzaldehyde (356-a)

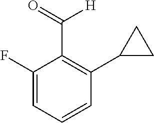 [SMILES: O=Cc1c(F)cccc1C1CC1]

To a solution of 2-bromo-6-fluoro-benzaldehyde (500 mg, 2.46 mmol) in toluene (2 mL) and H₂O (0.1 mL) was added cyclopropylboronic acid (317 mg, 3.69 mmol), K₃PO₄ (1.57 g, 7.39 mmol), and Pd(PPh₃)₄ (142 mg, 123 μmol) under nitrogen atmosphere. The mixture was degassed for three times, back filled with nitrogen, and allowed to stir at 120° C. for 2.5 hr. The reaction mixture was cooled to 25° C., quenched by water (10 mL) and extracted with EtOAc (20 mL×3). The organic solutions were combined, dried over anhydrous Na₂SO₄, and concentrated in vacuo to afford crude product. The crude product was purified by flash chromatography on silica gel column using 0-40% EtOAc/petroleum ether as eluent, to provide the title product 356-a as a light yellow oil (404 mg, 72.2% yield). LCMS (ESI) [M+H]⁺ m/z: 543.1.

### Step 2. 9-(2-cyclopropyl-6-fluorophenyl)-3,6,7,8-tetrahydro-2,3,5,5a,8a,10-hexaazabenzo[cd]cyclopenta[h]azulene (Compound 356)

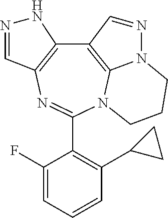 [SMILES: Fc1cccc(C2CC2)c1C1=Nc2cn[nH]c2-c2cnn3c2N1CCC3]

The title compound was made from intermediate 340-g (Example 4, Step 7), following synthetic procedure similar as described in the synthesis of compound 340 (Example 4, Steps 8-9), except that 356-a was used in the place of 2-fluoro-6-methyl-benzaldehyde. The crude product was purified by reverse phase preparative HPLC (column: Welch Xtimate C18 150×25 mm×5 μm; mobile phase: [water (10 mM NH₄HCO₃)-MeCN]; B %: 30%-55%, 10 min) to provide the title product 356 as a yellow solid (44 mg, 29.5%). MS (ESI) [M+H]⁺ m/z 349.1.

### Example 20. 9-(2,6-difluorophenyl)-7-methyl-2,6,7,8-tetrahydro-2,3,5,5a,8a,10-hexaazabenzo[cd]cyclopenta[h]azulene (Compound 357)

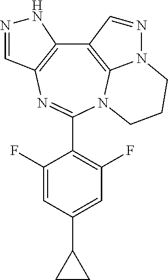 [SMILES: Fc1cc(C2CC2)cc(F)c1C1=Nc2cn[nH]c2-c2cnn3c2N1CCC3]

### Step 1. 4-cyclopropyl-2,6-difluorobenzaldehyde (357-a)

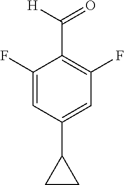 [SMILES: O=Cc1c(F)cc(C2CC2)cc1F]

A mixture of 4-bromo-2,6-difluoro-benzaldehyde (200 mg, 0.905 mmol), cyclopropylboronic acid (93.3 mg, 1.09 mmol), Na₂CO₃ (287 mg, 2.71 mmol), Pd(dppf)Cl₂ (66.2 mg, 90.5 μmol) in dioxane (5 mL) and H₂O (0.5 mL) was degassed and purged with N₂ for 3 times, and then the mixture was stirred at 100° C. for 16 hr under N₂ atmosphere. The crude product was purified by flash chromatography on silica gel column using 0-11% EtOAc/petroleum ether as eluent, to provide the title product 357-a as a yellow oil (130 mg, 72.5%).

### Step 2. 9-(2,6-difluorophenyl)-7-methyl-2,6,7,8-tetrahydro-2,3,5,5a,8a,10-hexaazabenzo[cd]cyclopenta[h]azulene (Compound 357)

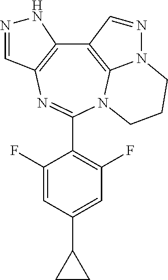 [SMILES: Fc1cc(C2CC2)cc(F)c1C1=Nc2cn[nH]c2-c2cnn3c2N1CCC3]

The title compound was made from intermediate 340-g (Example 4, Step 7), following synthetic procedure similar as described in the synthesis of compound 340 (Example 4, Steps 8-9), except that 357-a was used in the place of 2-fluoro-6-methyl-benzaldehyde. The crude product was purified by reverse phase preparative HPLC (column: Phenomenex Gemini-NX 80×40 mm×3 μm; mobile phase: [water (10 mM NH₄HCO₃)-MeCN]; B %: 23%-53%, 9.5 min) to provide the title product 357 as a yellow solid (56 mg, 57.2%). MS (ESI) [M+H]⁺ m/z 341.1.

### Example 21. 3,5-difluoro-4-[3-[(4-methoxyphenyl)methyl]-3,4,7,9,13,14-hexazatetracyclo [7.6.1.02,6.013,16]hexadeca-1(16),2(6),4,7,14-pentaen-8-yl] benzonitrile (Compound 358)

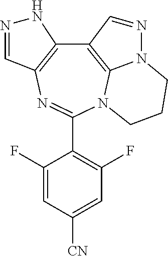 [SMILES: N#Cc1cc(F)c(C2=Nc3cn[nH]c3-c3cnn4c3N2CCC4)c(F)c1]

The title compound was made from intermediate 340-g (Example 4, Step 7), following synthetic procedure similar as described in the synthesis of compound 340 (Example 4, Steps 8-9), except that 3,5-difluoro-4-formyl-benzonitrile was used in the place of 2-fluoro-6-methyl-benzaldehyde. The crude product was purified by flash chromatography on silica gel column using 0-100% EtOAc/petroleum ether as eluent, to provide the title product 358 as a yellow oil (70 mg, 46.0%). MS (ESI) [M+H]⁺ m/z 352.1.

### Example 22. (3,5-difluoro-4-(3,6,7,8-tetrahydro-2,3,5,5a,8a,10-hexaazabenzo[cd]cyclopenta[h]azulen-9-yl)phenyl)methanol (Compound 359)

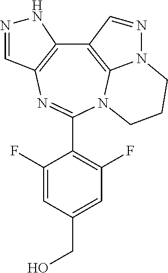 [SMILES: OCc1cc(F)c(C2=Nc3cn[nH]c3-c3cnn4c3N2CCC4)c(F)c1]

### Step 1. 2,6-difluoro-4-(hydroxymethyl)benzaldehyde (359-a)

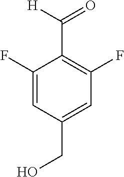 [SMILES: O=Cc1c(F)cc(CO)cc1F]

A solution of LDA/THF (2.5 M, 0.55 mL) was added to a −78° C. mixture of (3,5-difluorophenyl) methanol (200 mg, 1.39 mmol) in THF (2 mL) dropwise. The mixture was stirred at −78° C. for 30 min. DMF (1.39 mmol, 0.10 mL) was added into the mixture and the resulting solution was stirred at −78° C. for 1 hour. The reaction mixture was diluted with H₂O (10 mL) and the mixture was extracted with EtOAc (10 mL×3). The combined organic phase was washed with brine (10 mL×2), dried with anhydrous Na₂SO₄, filtered and concentrated in vacuum. The crude product was purified by flash chromatography on silica gel column using 0-60% EtOAc/petroleum ether as eluent to provide the title product 359-a as a yellow oil (90 mg, 37.6%).

### Step 2. (3,5-difluoro-4-(3,6,7,8-tetrahydro-2,3,5,5a,8a,10-hexaazabenzo[cd]cyclopenta[h]azulen-9-yl)phenyl)methanol (Compound 359)

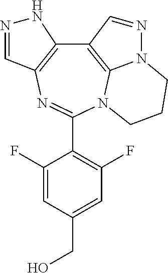 [SMILES: OCc1cc(F)c(C2=Nc3cn[nH]c3-c3cnn4c3N2CCC4)c(F)c1]

The title compound was made from intermediate 340-g (Example 4, Step 7), following synthetic procedure similar as described in the synthesis of compound 340 (Example 4, Steps 8-9), except that 359-a was used in the place of 2-fluoro-6-methyl-benzaldehyde. The crude product was purified by reverse phase preparative HPLC (column. Phenomenex Gemini-NX 80×40 mm×3 μm; mobile phase: [water (10 mM NH₄HCO₃)-MeCN]; B %: 13%-43%, 7.8 min) to provide the title product 359 as a yellow solid (3.9 mg, 20.4%). MS (ESI) [M+H]⁺ m/z 357.1.

### Example 23. 8-(2,6-difluoro-4-methoxy-phenyl)-3,4,7,9,13,14-hexazatetracyclo[7.6.1.02,6.013,16]hexadeca-1(16),2(6),4,7,14-pentaene (Compound 360)

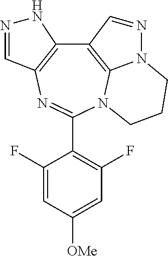 [SMILES: COc1cc(F)c(C2=Nc3cn[nH]c3-c3cnn4c3N2CCC4)c(F)c1]

The title compound was made from intermediate 340-g (Example 4, Step 7), following synthetic procedure similar as described in the synthesis of compound 340 (Example 4, Steps 8-9), except that 2,6-difluoro-4-methoxy-benzaldehyde was used in the place of 2-fluoro-6-methyl-benzaldehyde. The crude product was purified by reverse phase preparative HPLC (column: YMC-Triart Prep C18 150×40 mm×7 μm; mobile phase: [water (0.225% FA)-MeCN]; B %: 0%-40%, 9 min) to provide the title product 360 as a yellow solid (29.1 mg, 20.5%). MS (ESI) [M+H]⁺ m/z 357.2.

### Example 24. 8-phenyl-3,4,7,9,13,14-hexazatetracyclo[7.6.1.02,6.013,16]hexadeca-1(16),2(6),4,7,14-pentaene (Compound 361)

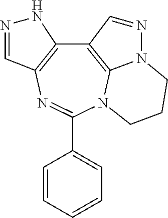 [SMILES: c1ccc(C2=Nc3cn[nH]c3-c3cnn4c3N2CCC4)cc1]

The title compound was made from intermediate 340-g (Example 4, Step 7), following synthetic procedure similar as described in the synthesis of compound 340 (Example 4, Steps 8-9), except that benzaldehyde was used in the place of 2-fluoro-6-methyl-benzaldehyde. The crude product was purified by reverse phase preparative HPLC (Column: Welch Xtimate C18 150×25 mm×5 μm; mobile phase: [water (10 mM NH₄HCO₃)-MeCN]; B %: 16%-46%, 10 min) to provide the title product 361 as a yellow solid (9.6 mg, 13.4%). MS (ESI) [M+H]⁺ m/z 291.1.

### Example 25. 9-(2-fluorophenyl)-3,6,7,8-tetrahydro-2,3,5,5a,8a,10-hexaazabenzo[cd]cyclopenta[h]azulene (Compound 362)

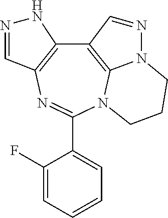 [SMILES: Fc1ccccc1C1=Nc2cn[nH]c2-c2cnn3c2N1CCC3]

The title compound was made from intermediate 340-g (Example 4, Step 7), following a synthetic procedure similar as described in the synthesis of compound 340 (Example 4, Steps 8-9), except that 2-fluorobenzaldehyde was used in the place of 2-fluoro-6-methylbenzaldehyde. The crude product was purified by flash chromatography (ISCO®; 4 g SepaFlash® Silica Flash Column, petroleum ether/EtOAc with EtOAc from 0˜100%, 30 mL/min, 254 nm), to provide the title compound 362 as a white solid (33.1 mg, 37.2%). MS (ESI) [M+H]⁺ m/z 309.1.

### Example 26. 10-(2-fluorophenyl)-6,7,8,9-tetrahydro-3H-2,3,5,5a,9a,11-hexaazadicyclopenta[b,kl]heptalene (Compound 363)

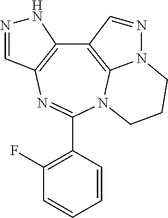 [SMILES: Fc1ccccc1C1=Nc2cn[nH]c2-c2cnn3c2N1CCC3]

The title compound was made from intermediate 344-n (Example 8, Step 14), following synthetic procedure similar as described in the synthesis of compound 340 (Example 4, Steps 8-9), except that 2-fluorobenzaldehyde was used in the place of 2-fluoro-6-methylbenzaldehyde. The crude product was purified by reverse phase preparative HPLC (column: Phenomenex Gemini-NX 80×40 mm×3 μm; mobile phase: [water (10 mM NH₄HCO₃)-MeCN]; B %: 20%-50%, 7.8 min), to provide the title compound 363 as a yellow solid (8.0 mg, 22.9%). MS (ESI) [M+H]⁺ m/z 323.1.

### Example 27. 10-(2-chlorophenyl)-6,7,8,9-tetrahydro-3H-2,3,5,5a,9a,11-hexaazadicyclopenta[b,kl]heptalene (Compound 364)

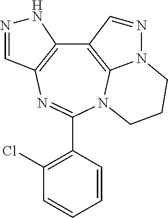 [SMILES: Clc1ccccc1C1=Nc2cn[nH]c2-c2cnn3c2N1CCC3]

The title compound was made from intermediate 344-n (Example 8, Step 14), following synthetic procedure similar as described in the synthesis of compound 340 (Example 4, Steps 8-9), except that 2-chlorobenzaldehyde was used in the place of 2-fluoro-6-methylbenzaldehyde. The crude product was purified by reverse phase preparative HPLC (column: Waters Xbridge BEH C18 150×25 mm×5 μm; mobile phase: [water (0.225% FA)-MeCN]; B %: 0%-50%, 7.8 min), to provide the title compound 364 as a yellow solid (7.03 mg, 84.7%). MS (ESI) [M+H]⁺ m/z 338.9.

### Example 28. 10-(3-fluoropyridin-2-yl)-6,7,8,9-tetrahydro-3H-2,3,5,5a,9a,11-hexaazadicyclopenta[b,kl]heptalene (Compound 365)

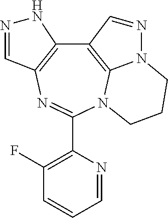 [SMILES: Fc1cccnc1C1=Nc2cn[nH]c2-c2cnn3c2N1CCC3]

The title compound was made from intermediate 344-n (Example 8, Step 14), following synthetic procedure similar as described in the synthesis of compound 340 (Example 4, Steps 8-9), except that 3-fluoropyridine-2-carbaldehyde was used in the place of 2-fluoro-6-methylbenzaldehyde. The crude product was purified by reverse phase preparative HPLC (column: Waters Xbridge BEH C18 150×25 mm×5 μm; mobile phase: [water (0.225% FA)-MeCN]; B %: 0%-45%, 7.8 min), to provide the title compound 365 as a yellow solid (4.0 mg, 56.8%). MS (ESI) [M+H₃O]⁺ m/z 342.1.

### Example 29. 9-(2,6-difluorophenyl)-4-methyl-3,6,7,8-tetrahydro-2,3,5,5a,8a,10-hexaazabenzo[cd]cyclopenta[h]azulene (Compound 366)

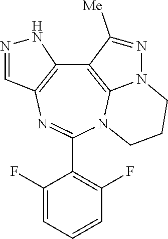 [SMILES: Cc1nn2c3c1-c1[nH]ncc1N=C(c1c(F)cccc1F)N3CCC2]

The title compound was made from 3-methyl-1H-pyrazol-5-amine, following synthetic procedure similar as described in the synthesis of compound 340 (Example 4, Steps 2-9), except that 3-methyl-1H-pyrazol-5-amine was used in the place of 1H-pyrazol-5-amine, and that, 2,6-difluorobenzaldehyde was used in the place of 2-fluoro-6-methyl-benzaldehyde. The crude product was purified by preparative TLC (silica, petroleum ether/EtOAc=0/1, 254 nm), to provide the title compound 366 as a yellow solid (2.31 mg, 42.1%). MS (ESI) [M+H]⁺ m/z 341.1.

### Example 30. 8-(2,3,6-trifluorophenyl)-3,4,7,9,13,14-hexazatetracyclo[7.6.1.02,6.013,16]hexadeca-1(16),2(6),4,7,14-pentaene (Compound 367)

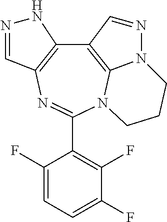 [SMILES: Fc1ccc(F)c(C2=Nc3cn[nH]c3-c3cnn4c3N2CCC4)c1F]

The title compound was made from intermediate 340-g (Example 4, Step 7), following synthetic procedure similar as described in the synthesis of compound 340 (Example 4, Steps 8-9), except that 2,3,6-trifluorobenzaldehyde was used in the place of 2-fluoro-6-methylbenzaldehyde. The crude product was purified by reverse phase preparative HPLC (column: Welch Xtimate C18 150×25 mm×5 μm; mobile phase: [water (0.225% FA)-MeCN]; B %: 20%-50%, 7.8 min, 254 nm), to provide the title compound 367 as a yellow solid (4.97 mg, 4.4%). MS (ESI) [M+H]⁺ m/z 345.1.

### Example 31. 8-(3-chloro-2,6-difluoro-phenyl)-3,4,7,9,13,14-hexazatetracyclo[7.6.1.02,6.013,16]hexadeca-1(16),2(6),4,7,14-pentaene (Compound 368)

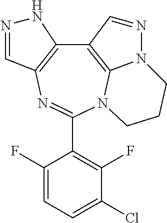 [SMILES: Fc1ccc(Cl)c(F)c1C1=Nc2cn[nH]c2-c2cnn3c2N1CCC3]

The title compound was made from intermediate 340-g (Example 4, Step 7), following synthetic procedure similar as described in the synthesis of compound 340 (Example 4, Steps 8-9), except that 3-chloro-2,6-difluorobenzaldehyde was used in the place of 2-fluoro-6-methylbenzaldehyde. The crude product was purified by reverse phase preparative HPLC (column: Welch Xtimate C18 150×25 mm×5 μm; mobile phase: [water (0.225% FA)-MeCN]; B %: 20%-50%, 7.8 min), to provide the title compound 368 as a yellow solid (4.67 mg, 10.2%). MS (ESI) [M+H]⁺ m/z 361.1.

### Example 32. 9-(2,6-difluorophenyl)-3,6,7,8-tetrahydro-2,3,3b,4,8a,10-hexaazabenzo[cd]cyclopenta[h]azulene (Compound 369)

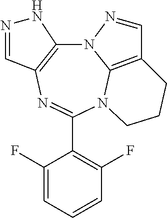 [SMILES: Fc1cccc(F)c1C1=Nc2cn[nH]c2-n2ncc3c2N1CCC3]

### Step 1. 5-iodo-1-(4-methoxybenzyl)-4-nitro-1H-pyrazole (369-a)

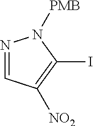

In to a −78° C. solution of intermediate 340-a (Example 4, Step 1, 5 g, 21.4 mmol) in THE (100 mL) under N₂ atmosphere was added LiHMDS/THF (1M, 23.6 mL, 23.6 mmol) dropwise in 3 portions. The mixture was stirred at −78° C. for 30 minutes, then NIS (5.31 g, 23.6 mmol) was added. The mixture was allowed to warm to 25° C. gradually and stirred for 2 hr. The reaction mixture was quenched with saturated NH₄Cl/H₂O (100 mL), extracted with EtOAc (50 mL×2).

The combined organic layers were washed with brine (50 mL), dried over Na₂SO₄ and concentrated in vacuum. The crude product was purified by flash chromatography (silica, eluted with EtOAc in petroleum ether from 0 to 30%, 254 nm) to give the title compound 369-a as a yellow solid (4 g, 51.9%).

### Step 2. 1-(1-(4-methoxybenzyl)-4-nitro-1H-pyrazol-5-yl)-1H-pyrazolo[3,4-b]pyridine (369-b)

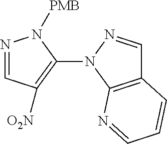

Intermediate 369-a (3.1 g, 8.63 mmol) and 1H-pyrazolo[3,4-b]pyridine (925.5 mg, 7.77 mmol) were dissolved in NMP (50 mL). Then K₂CO₃ (2.39 g, 17.3 mmol) was added and the mixture was stirred at 120° C. for 12 hr. The reaction mixture was quenched with EtOAc (50 mL) and H₂O (100 mL). The organic layer was washed with H₂O (100 mL×3), dried over Na₂SO₄, filtered and concentrated in vacuum. The crude product was purified by flash chromatography (Biotage reversed C18 column, eluted with water MeCN in H₂O from 0 to 100%, 254 nm) to give the compound 369-b as a yellow solid (520 mg, 17.2%). MS (ESI) [M+H]⁺ m/z 350.9.

### Step 3. 1-(4-methoxybenzyl)-5-(4,5,6,7-tetrahydro-1H-pyrazolo[3,4-b]pyridin-1-yl)-1H-pyrazol-4-amine (369-c)

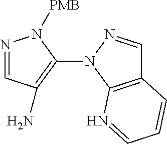

A stirred solution of 369-b (520 mg) in MeOH (5 mL) was added with Pt/C (62.37 mg, 14.8 umol, 5% purity) was added under Ar. The mixture was purged and degassed with N₂ for 3 times, then purged and degassed with H₂ for 3 times, then stirred under 20° C. under 40 psi for 16 hr. The reaction mixture was filtered via a cake of celite. The cake was washed with MeOH (10 mL). The filtrate was concentrated in vacuum to give the title compound 369-c as a yellow solid (400 mg). MS (ESI) [M+H]⁺ m/z 325.1.

### Step 4. 9-(2,6-difluorophenyl)-3,6,7,8-tetrahydro-2,3,3b,4,8a,10-hexaazabenzo[cd]cyclopenta[h]azulene (Compound 369)

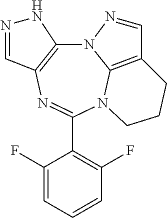 [SMILES: Fc1cccc(F)c1C1=Nc2cn[nH]c2-n2ncc3c2N1CCC3]

The title compound was made from intermediate 369-c (Example 32, Step 3), following synthetic procedure similar as described in the synthesis of compound 340 (Example 4, Steps 8-9), except that 2,6-difluorobenzaldehyde was used in the place of 2-fluoro-6-methyl-benzaldehyde. The crude product was purified by reverse phase preparative HPLC (column: Welch Xtimate C18 150×25 mm×5 μm; mobile phase: [water (10 mM NH₄HCO₃)-MeCN]; B %: 17%-47%, 10 min), to provide the title compound 369 as a yellow solid (2.01 mg, 9.2%). MS (ESI) [M+H]⁺ m/z 327.0.

### Example 33. 9-(2,6-difluorophenyl)-1-methyl-3,6,7,8-tetrahydro-2,3,5,5a,8a,10-hexaazabenzo[cd]cyclopenta[h]azulene (Compound 370)

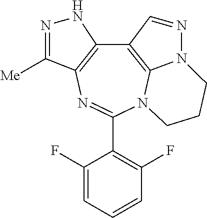 [SMILES: Cc1n[nH]c2c1N=C(c1c(F)cccc1F)N1CCCn3ncc-2c31]

### Step 1: 1-(4-methoxybenzyl)-3-methyl-4-nitro-1H-pyrazole (370-a)

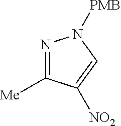

The title compound was made from 3-methyl-4-nitro-1H-pyrazole, following synthetic procedure similar as described in the synthesis of compound 340-a (Example 4, Step 1), except that 3-methyl-4-nitro-1H-pyrazole was used in the place of 4-nitro-1H-pyrazole. The crude product was purified by column chromatography (silica, DCM/MeOH=100/1, 254 nm), to provide the title compound 370-a as a pale yellow solid (36.4 g, 40.9%).

### Step 2. 9-(2,6-difluorophenyl)-1-methyl-3,6,7,8-tetrahydro-2,3,5,5a,8a,10-hexaazabenzo[cd]cyclopenta[h]azulene (Compound 370)

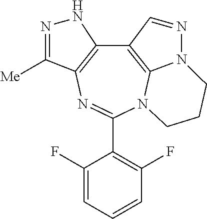 [SMILES: Cc1n[nH]c2c1N=C(c1c(F)cccc1F)N1CCCn3ncc-2c31]

The title compound was made from intermediate 370-a (Example 33, step 1) and 340-d (Example 97, step 4), following synthetic procedure similar as described in the synthesis of compound 340 (Example 4, Steps 5-9), except that 2,6-difluorobenzaldehyde was used in the place of 2-fluoro-6-methyl-benzaldehyde. The crude product was reverse phase preparative HPLC (column: YMC Triart C18 250×50 mm×7 μm; mobile phase: [water (0.225% FA)-MeCN]; B %: 8%-48%, 9 min), to provide the title compound 370 as a yellow solid (21.7 mg, 29.3%). MS (ESI) [M+H]⁺ m/z 341.1.

### Example 34. 5-chloro-8-(2,6-difluorophenyl)-3,4,7,9,13,14-hexazatetracyclo[7.6.1.02,6.013,16]hexadeca-1(16),2(6),4,7,14-pentaene (Compound 371)

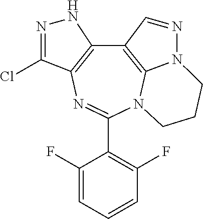 [SMILES: Fc1cccc(F)c1C1=Nc2c(Cl)n[nH]c2-c2cnn3c2N1CCC3]

To a solution of 9-(2,6-difluorophenyl)-7,8-dihydro-3H,6H-2,3,5,5a,8a,10-hexaazabenzo[cd]cyclopenta[h]azulene (Example 65, Step 5, 30 mg, 91.9 μmol) in MeCN (1 mL) was added NCS (12.2 mg, 91.9 umol). The mixture was stirred at 60° C. for 16 hr. The reaction mixture was concentrated under reduced pressure and the residue was purified by reverse phase HPLC (column: Welch Xtimate C18 150×25 mm×5 μm; mobile phase: [water (0.225% FA)-MeCN]; B %: 28%-58%, 9.5 min) to afford the title compound 371 as a yellow solid (2.57 mg, 7.6%). MS (ESI) [M+H]⁺ m/z 361.1.

### Example 35. 8-(2,6-difluorophenyl)-5-methyl-3,4,7,9,12,13-hexazatetracyclo[7.5.1.02,6.012,15]pentadeca-1(15),2,5,7,13-pentaene (Compound 372)

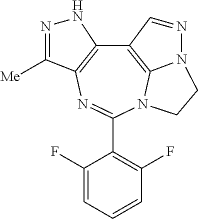 [SMILES: Cc1n[nH]c2c1N=C(c1c(F)cccc1F)N1CCn3ncc-2c31]

### Step 1. 2,3-dihydro-1H-imidazo[1,2-b]pyrazole (372-a)

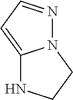

The title compound was made from 1H-pyrazol-5-amine, following synthetic procedure similar as described in the synthesis of compound 340-b (Example 4, Step 2), except that 1,2-dibromoethane was used in the place of 1,3-dibromopropane. The crude product was purified by flash chromatography (ISCO®; 40 g SepaFlash® Silica Flash Column, petroleum ether/EtOAc with EtOAc from 0˜100%, 45 mL/min, 254 nm), to provide the title compound 372-a as a yellow oil (1.2 g, 3.6%).

### Step 2. tert-butyl 7-(1-(4-methoxybenzyl)-3-methyl-4-nitro-1H-pyrazol-5-yl)-2,3-dihydro-1H-imidazo[1,2-b]pyrazole-1-carboxylate (372-b)

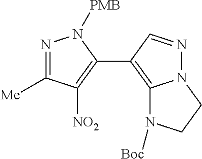

The title compound was made from intermediate 372-a (Example 35, Step 1), following synthetic procedure similar as described in the synthesis of compound 340-e (Example 4, Steps 3-5), except 370-a was used in the place of 340-a. The crude product was purified by flash chromatography (ISCO®; 40 g SepaFlash® Silica Flash Column, petroleum ether/EtOAc with EtOAc from 0˜50%, 45 mL/min, 254 nm), to provide the title compound 372-b as a yellow oil (730 mg, 64.0%). MS (ESI) [M+H]⁺ m/z 455.2.

### Step 3. 5-(2,3-dihydro-1H-imidazo[1,2-b]pyrazol-7-yl)-1-(4-methoxybenzyl)-3-methyl-1H-pyrazol-4-amine (372-c)

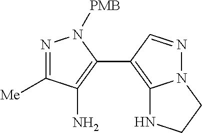

The title compound was made from intermediate 372-b (Example 35, Step 2), following synthetic procedure similar as described in the synthesis of compound 340-g (Example 4, Steps 6-7). The crude product was concentrated, to provide the title compound 372-c as a yellow oil (500 mg, crude). MS (ESI) [M+H]⁺ m/z 325.1.

### Step 4. 8-(2,6-difluorophenyl)-5-methyl-3,4,7,9,12,13-hexazatetracyclo[7.5.1.02,6.012,15]pentadeca-1(15),2,5,7,13-pentaene (Compound 372)

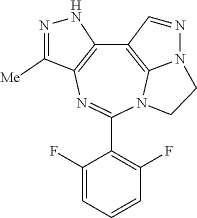 [SMILES: Cc1n[nH]c2c1N=C(c1c(F)cccc1F)N1CCn3ncc-2c31]

The title compound was made from intermediate 372-c (Example 35, Step 3), following synthetic procedure similar as described in the synthesis of compound 340 (Example 4, Steps 8-9), except that 2,6-difluorobenzaldehyde was used in the place of 2-fluoro-6-methyl-benzaldehyde. The crude product was purified by flash chromatography (ISCO®; 12 g SepaFlash® Silica Flash Column, petroleum ether/EtOAc with EtOAc from 0˜100%, 35 mL/min, 254 nm), to provide the title compound 372 as a yellow oil (60 mg, 26.3%). MS (ESI) [M+H]⁺ m/z 327.1.

### Example 36. 3-(2,6-difluorophenyl)-2,7-dihydro-1H-2a,4,6,7,9,9a-hexaazadicyclopenta[cd,f]azulene (Compound 373)

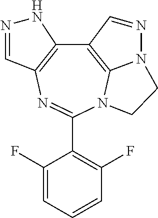 [SMILES: Fc1cccc(F)c1C1=Nc2cn[nH]c2-c2cnn3c2N1CC3]

### Step 1. tert-butyl 7-(1-(4-methoxybenzyl)-4-nitro-1H-pyrazol-5-yl)-2,3-dihydro-1H-imidazo[1,2-b]pyrazole-1-carboxylate (373-a)

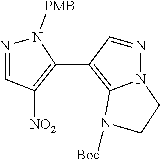

The title compound was made from intermediate 372-a (Example 35, Step 1), following synthetic procedure similar as described in the synthesis of compound 340-e (Example 4, Steps 3-5), except that 340-a was used in the place of 370-a. The crude product was purified by flash chromatography (ISCO®; 80 g SepaFlash® Silica Flash Column, petroleum ether/EtOAc with EtOAc from 0˜50%, 50 mL/min, 254 nm), to provide the title compound 373-a as a yellow oil (3.5 g, 47.9%). MS (ESI) [M+H]⁺ m/z 441.2.

### Step 2. 3-(2,6-difluorophenyl)-2,7-dihydro-1H-2a,4,6,7,9,9a-hexaazadicyclopenta[cd,f]azulene (Compound 373)

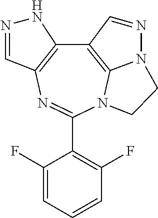 [SMILES: Fc1cccc(F)c1C1=Nc2cn[nH]c2-c2cnn3c2N1CC3]

The title compound was made from intermediate 373-a (Example 36, Step 1), following synthetic procedure similar as described in the synthesis of compound 340 (Example 4, Steps 6-9). The crude product was purified by flash chromatography (ISCO®; 20 g SepaFlash® Silica Flash Column, petroleum ether/EtOAc with EtOAc from 0˜100%, 40 mL/min, 254 nm), to provide the title compound 373 as a yellow solid (260 mg, 98.8%). MS (ESI) [M+H]⁺ m/z 313.1.

### Example 37. 8-(2,6-difluorophenyl)-14-methyl-1,3,4,7,9,15-hexazatetracyclo[7.6.1.02,6.013,16]hexadeca-2(6),4,7,13(16),14-pentaene (Compound 374)

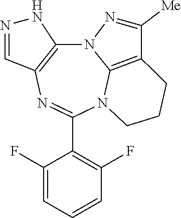 [SMILES: Cc1nn2c3c1CCCN3C(c1c(F)cccc1F)=Nc1cn[nH]c1-2]

### Step 1. 1-(1-(4-methoxybenzyl)-4-nitro-1H-pyrazol-5-yl)-3-methyl-1H-pyrazolo[3,4-b]pyridine (374-a)

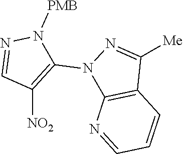

The title compound was made from intermediate 369-a (Example 32, Step 1), following synthetic procedure similar as described in the synthesis of compound 369-b (Example 32, Step 2), except that 3-methyl-1H-pyrazolo[3,4-b]pyridine was used in the place of 1H-pyrazolo[3,4-b]pyridine. The crude product was purified by flash chromatography (ISCO®; 12 g SepaFlash® Silica Flash Column, petroleum ether/EtOAc with EtOAc from 0˜20%, 35 mL/min, 254 nm), to provide the title compound 374-a as a yellow oil (418 mg, 13.9%). MS (ESI) [M+H]⁺ m/z 365.1.

### Step 2. 1-(4-methoxybenzyl)-5-(3-methyl-1H-pyrazolo[3,4-b]pyridin-1-yl)-1H-pyrazol-4-amine (374-b)

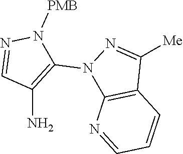

The title compound was made from intermediate 374-a (Example 37, Step 1), following synthetic procedure similar as described in the synthesis of compound 369-c (Example 32, Step 3). The crude product was purified by flash chromatography (ISCO®; 12 g SepaFlash® Silica Flash Column, petroleum ether/EtOAc with EtOAc from 0˜100%, 35 mL/min, 254 nm), to provide the title compound 374-b as yellow oil (122 mg, 63.7%).

### Step 3. 8-(2,6-difluorophenyl)-14-methyl-1,3,4,7,9,15-hexazatetracyclo[7.6.1.02,6.013,16]hexadeca-2(6),4,7,13(16),14-pentaene (Compound 374)

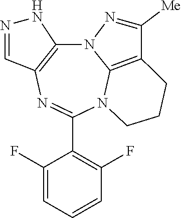 [SMILES: Cc1nn2c3c1CCCN3C(c1c(F)cccc1F)=Nc1cn[nH]c1-2]

The title compound was made from intermediate 374-b (Example 37, Step 2), following synthetic procedure similar as described in the synthesis of compound 340 (Example 4, Steps 8-9), except that 2,6-difluorobenzaldehyde was used in the place of 2-fluoro-6-methyl-benzaldehyde. The crude product was purified by reverse phase preparative HPLC (column: 3_Phenomenex Luna C18 75×30 mm×3 μm; mobile phase: [water (0.225% FA)-MeCN]; B %: 20%-50%, 6.5 min), to provide the title compound 374 as a yellow oil (23.45 mg, 33.2%). MS (ESI) [M+H]⁺ m/z 341.1.

### Example 38. 5-chloro-3-(2,6-difluorophenyl)-2,7-dihydro-1H-2a,4,6,7,9,9a-hexaazadicyclopenta[cd,f]azulene (Compound 375)

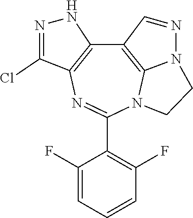 [SMILES: Fc1cccc(F)c1C1=Nc2c(Cl)n[nH]c2-c2cnn3c2N1CC3]

### Step 1. tert-butyl 7-(3-chloro-4-nitro-1-((2-(trimethylsilyl)ethoxy)methyl)-1H-pyrazol-5-yl)-2,3-dihydro-1H-imidazo[1,2-b]pyrazole-1-carboxylate (375-a)

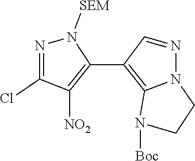 [SMILES: CC(C)(C)OC(=O)N1CCn2ncc(-c3c([N+](=O)[O-])c(Cl)nn3COCC[Si](C)(C)C)c21]

2-[(3-chloro-4-nitro-pyrazol-1-yl)methoxy]ethyl-trimethyl-silane (828.8 mg, 2.98 mmol), tert-butyl 7-iodo-2,3-dihydroimidazo[1,2-b]pyrazole-1-carboxylate (1 g, 2.98 mmol), Cs₂CO₃ (1.46 g, 4.48 mmol) and cesium; 2,2-dimethylpropanoate (907.8 mg, 3.88 mmol) were dissolved in dry toluene (25 mL). The reaction mixture was degassed with nitrogen under sonication, followed by the addition of Pd(OAc)₂ (66.9 mg, 0.298 mmol), XPhos (426.7 mg, 0.895 mmol). The mixture was then degassed again and stirred 40 hr at 120° C. The reaction mixture was diluted with H₂O (30 mL) and extracted with EtOAc (30 mL×3). The combined organic layers were washed with brine (30 mL×2), dried with anhydrous Na₂SO₄, filtered and concentrated under reduced pressure. The residue was purified by flash chromatography (ISCO®; 20 g SepaFlash® Silica Flash Column, petroleum ether/EtOAc with EtOAc from 0˜30%, 30 mL/min, 254 nm) to give the title compound 375-a as a yellow oil (737 mg, 45.8%). MS (ESI) [M+H]⁺ m/z 485.1.

### Step 2. tert-butyl 7-(4-amino-3-chloro-1-((2-(trimethylsilyl)ethoxy)methyl)-1H-pyrazol-5-yl)-2,3-dihydro-1H-imidazo[1,2-b]pyrazole-1-carboxylate (375-b)

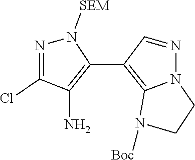 [SMILES: CC(C)(C)OC(=O)N1CCn2ncc(-c3c(N)c(Cl)nn3COCC[Si](C)(C)C)c21]

To a solution of 375-a (600 mg, 1.24 mmol) in THE (6 mL) was added NiCl₂·6H₂O (1.76 g, 7.42 mmol) and NaBH₄ (280.8 mg, 7.42 mmol). The mixture was stirred at 25° C. for 1 hour. The reaction mixture was diluted with H₂O (30 mL) and extracted with EtOAc (30 mL×3). The combined organic layers were washed with brine (30 mL×2), dried with anhydrous Na₂SO₄, filtered and concentrated under reduced pressure. The residue was purified by flash chromatography (ISCO®; 24 g SepaFlash® Silica Flash Column, petroleum ether/EtOAc with EtOAc from 0˜80%, 100 mL/min, 254 nm) to give the title compound 375-b as a yellow oil (372 mg, 66.0%). MS (ESI) [M+H]⁺ m/z 455.2.

### Step 3. 3-chloro-5-(2,3-dihydro-1H-imidazo[1,2-b]pyrazol-7-yl)-1H-pyrazol-4-amine (375-c)

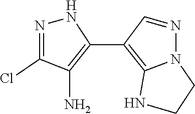 [SMILES: Nc1c(Cl)n[nH]c1-c1cnn2c1NCC2]

To a solution of 375-b (335 mg, 0.736 mmol) in 4M HCl/EtOAc (10 mL). The mixture was stirred at 60° C. for 16 hr. The reaction mixture was concentrated under reduced pressure to remove HCl/EtOAc to give the title compound 375-c as a yellow oil (165 mg, crude).

### Step 4. 5-chloro-3-(2,6-difluorophenyl)-2,7-dihydro-1H-2a,4,6,7,9,9a-hexaazadicyclopenta[cd,f]azulene (Compound 375)

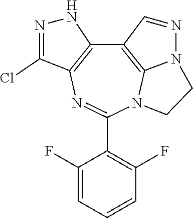 [SMILES: Fc1cccc(F)c1C1=Nc2c(Cl)n[nH]c2-c2cnn3c2N1CC3]

The title compound was made from intermediate 375-c (Example 38, Step 3), following synthetic procedure similar as described in the synthesis of compound 340 (Example 4, Steps 8-9), except that 2,6-difluorobenzaldehyde was used in the place of 2-fluoro-6-methyl-benzaldehyde. The crude product was purified by flash chromatography (ISCO®; 4 g SepaFlash® Silica Flash Column, petroleum ether/EtOAc with EtOAc from 0˜100%, 30 mL/min, 254 nm) and preparative TLC (silica, petroleum ether/EtOAc=0/1, 254 nm), to provide the title compound 375 as a yellow oil (20.32 mg, 7.6%). MS (ESI) [M+H]⁺ m/z 347.1.

### Example 39. 8-(3,5-difluoro-4-pyridyl)-3,4,7,9,13,14-hexazatetracyclo[7.6.1.02,6.013,16]hexadeca-1(16),2(6),4,7,14-pentaene (Compound 376)

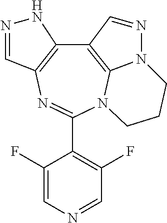 [SMILES: Fc1cncc(F)c1C1=Nc2cn[nH]c2-c2cnn3c2N1CCC3]

The title compound was made from intermediate 340-g (Example 4, Step 7), following synthetic procedure similar as described in the synthesis of compound 340 (Example 4, Steps 8-9), except that 3,5-difluoropyridine-4-carbaldehyde was used in the place of 2-fluoro-6-methylbenzaldehyde. The crude product was purified by reverse phase preparative HPLC (column: 3_Phenomenex Luna C18 75×30 mm×3 μm; mobile phase: [water (0.225% FA)-MeCN]; B %: 5%-35%, 6.5 min), to provide the title compound 376 as a yellow solid (16.69 mg, 30.2%). MS (ESI) [M+H]⁺ m/z 328.1.

### Example 40. 9-(2,6-difluorophenyl)-6,8-dihydro-3H-spiro[2,3,5,5a,8a,10-hexaazabenzo[cd]cyclopenta[h]azulene-7,1′-cyclopropane] (Compound 377)

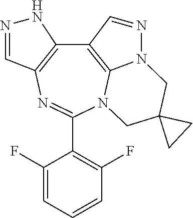 [SMILES: Fc1cccc(F)c1C1=Nc2cn[nH]c2-c2cnn3c2N1CC1(CC1)C3]

### Step 1: 4′,5′-dihydro-7′H-spiro[cyclopropane-1,6′-pyrazolo[1,5-a]pyrimidine] (377-a)

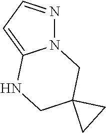 [SMILES: c1cc2n(n1)CC1(CC1)CN2]

A stirred solution of 1H-pyrazol-5-amine (7.0 g, 84.2 mmol) in dioxane (280 mL) was added with TEA (21.3 g, 210.6 mmol) 1,1-bis(bromomethyl)cyclopropane (19.2 g, 84.2 mmol). The reaction mixture was stirred at 100° C. for 16 hr under nitrogen. The reaction mixture was added EtOAc (100 mL) and filtered to remove the insoluble. The filter liquor was concentrated in vacuo to give residue. The residue was purified by flash chromatography (ISCO®; 80 g SepaFlash® Silica Flash Column, DCM/EtOAc with EtOAc from 0˜100%, 50 mL/min, 254 nm) to give the title compound 377-a as a light yellow oil (3.0 g, 23.9%).

### Step 2: tert-butyl 3′-(4-nitro-1-((2-(trimethylsilyl)ethoxy)methyl)-1H-pyrazol-5-yl)-7′H-spiro[cyclopropane-1,6′-pyrazolo[1,5-a]pyrimidine]-4′(5′H)-carboxylate (377-b)

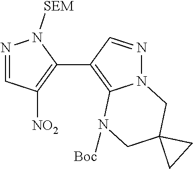 [SMILES: CC(C)(C)OC(=O)N1CC2(CC2)Cn2ncc(-c3c([N+](=O)[O-])cnn3COCC[Si](C)(C)C)c21]

The title compound was made from intermediate 377-a (Example 40, Step 1), following synthetic procedure similar as described in the synthesis of compound 340-e (Example 4, Steps 3-5), except that 4-nitro-1-((2-(trimethylsilyl)ethoxy)methyl)-1H-pyrazole was used in the place of 340-a. The crude product was purified by flash chromatography (ISCO®; 80 g SepaFlash® Silica Flash Column, petroleum ether/EtOAc with EtOAc from 0˜50%, 50 mL/min, 254 nm), to provide the title compound 377-b as a yellow oil (2 g, 53.5%). MS (ESI) [M+H]⁺ m/z 491.2.

### Step 3: 9-(2,6-difluorophenyl)-6,8-dihydro-3H-spiro[2,3,5,5a,8a,10-hexaazabenzo[cd]cyclopenta[h]azulene-7,1′-cyclopropane] (Compound 377)

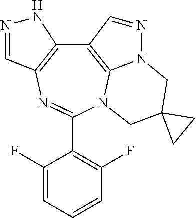 [SMILES: Fc1cccc(F)c1C1=Nc2cn[nH]c2-c2cnn3c2N1CC1(CC1)C3]

The title compound was made from 377-b (Example 40, Step 2), following synthetic procedure similar as described in the synthesis of compound 375 (Example 38, Steps 2-4), except that 2,6-difluorobenzaldehyde was used in the place of 2-fluoro-6-methylbenzaldehyde. The crude product was purified by flash chromatography (ISCO®; 25 g SepaFlash® Silica Flash Column, petroleum ether/EtOAc with EtOAc from 0˜100%, 50 mL/min, 254 nm) and further purified by preparative TLC (silica, DCM/MeOH=10/1, 254 nm) to provide the title compound 377 as a yellow oil (9 mg, 3.9%). MS (ESI) [M+H]⁺ m/z 353.1.

### Example 41. 8-(2,6-dimethylphenyl)-3,4,7,9,13,14-hexazatetracyclo[7.6.1.02,6.013,16]hexadeca-1(16),2(6),4,7,14-pentaene (Compound 379)

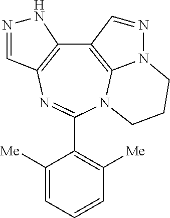 [SMILES: Cc1cccc(C)c1C1=Nc2cn[nH]c2-c2cnn3c2N1CCC3]

The title compound was made from intermediate 340-g (Example 4, Step 7), following synthetic procedure similar as described in the synthesis of compound 340 (Example 4, Steps 8-9), except that 2,6-dimethylbenzaldehyde was used in the place of 2-fluoro-6-methylbenzaldehyde. The crude product was purified by reverse phase preparative HPLC (Boston Prime C18 150×30 mm×5 μm; mobile phase: [water (ammonia hydroxide v/v)-MeCN]; B %: 13%-43%, 9 min), to provide the title compound 379 as a yellow solid (3 mg, 2.6%). MS (ESI) [M+H]⁺ m/z 319.1.

### Example 42. 8-[2-fluoro-6-(trifluoromethyl)phenyl]-3,4,7,9,13,14-hexazatetracyclo[7.6.1.02,6.013,16]hexadeca-1(16),2(6),4,7,14-pentaene (Compound 380)

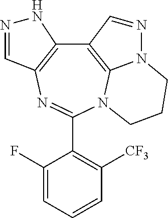 [SMILES: Fc1cccc(C(F)(F)F)c1C1=Nc2cn[nH]c2-c2cnn3c2N1CCC3]

The title compound was made from intermediate 340-g (Example 4, Step 7), following synthetic procedure similar as described in the synthesis of compound 340 (Example 4, Steps 8-9), except that 2-fluoro-6-(trifluoromethyl)benzaldehyde was used in the place of 2-fluoro-6-methylbenzaldehyde. The crude product was purified by reverse phase preparative HPLC (Boston Prime C18 150×30 mm×5 μm; mobile phase: [water (ammonia hydroxide v/v)-MeCN]; B %: 13%-43%, 9 min), to provide the title compound 380 as a yellow solid (3 mg, 2.6%). MS (ESI) [M+H]⁺ m/z 319.1.

### Example 43. 8-[2-(difluoromethyl)-6-fluoro-phenyl]-3,4,7,9,13,14-hexazatetracyclo[7.6.1.02,6.013,16]hexadeca-1(16),2(6),4,7,14-pentaene (Compound 381)

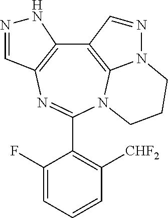 [SMILES: Fc1cccc(C(F)F)c1C1=Nc2cn[nH]c2-c2cnn3c2N1CCC3]

The title compound was made from intermediate 340-g (Example 4, Step 7), following synthetic procedure similar as described in the synthesis of compound 340 (Example 4, Steps 8-9), except that 2-(difluoromethyl)-6-fluoro-benzaldehyde was used in the place of 2-fluoro-6-methylbenzaldehyde. The crude product was purified by preparative TLC (silica, petroleum ether/EtOAc=1/1, 254 nm), to provide the title compound 381 as a yellow solid (15.1 mg, 7.8%). MS (ESI) [M+H]⁺ m/z 359.2.

### Example 44. 8-(2-fluoro-6-methyl-phenyl)-3,4,7,9,12,13-hexazatetracyclo[7.5.1.02,6.012,15]pentadeca-1(15),2(6),4,7,13-pentaene (Compound 382)

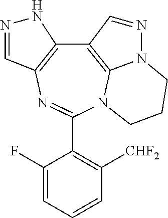 [SMILES: Fc1cccc(C(F)F)c1C1=Nc2cn[nH]c2-c2cnn3c2N1CCC3]

The title compound was made from intermediate 373-a (Example 36, Step 1), following synthetic procedure similar as described in the synthesis of compound 340 (Example 4, Steps 6-9). The crude product was purified by reverse phase preparative HPLC (column: Phenomenex Gemini-NX 150×30 mm×5 μm; mobile phase: [water (0.225% FA)-MeCN]; B %: 6%-46%, 9 min), to provide the title compound 382 as a yellow solid (55.3 mg, 38.0%). MS (ESI) [M+H]⁺ m/z 323.1.

### Example 45. 8-(2-chloro-6-fluoro-phenyl)-3,4,7,9,12,13-hexazatetracyclo[7.5.1.02,6.012,15]pentadeca-1(15),2(6),4,7,13-pentaen (Compound 383)

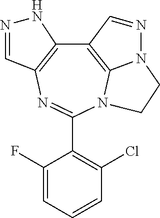 [SMILES: Fc1cccc(Cl)c1C1=Nc2cn[nH]c2-c2cnn3c2N1CC3]

The title compound was made from intermediate 373-a (Example 36, Step 1), following synthetic procedure similar as described in the synthesis of compound 340 (Example 4, Steps 6-9), except that 2-chloro-6-fluoro-benzaldehyde was used in the place of 2-fluoro-6-methylbenzaldehyde. The crude product was purified by reverse phase preparative HPLC (column: 3_Phenomenex Luna C18 75×30 mm×3 μm; mobile phase: [water (0.225% FA)-MeCN]; B %: 51%-35%, 7.8 min), to provide the title compound 383 as a yellow solid (37.4 mg, 23.1%). MS (ESI) [M+H]⁺ m/z 329.0.

### Example 46. 3-(4-(difluoromethoxy)-2,6-difluorophenyl)-9-(trifluoromethyl)-1,7-dihydropyrazolo[3′,4′:6,7]azepino[3,4,5-cd]indole (Compound 384)

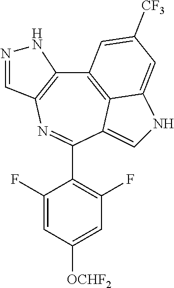 [SMILES: Fc1cc(OC(F)F)cc(F)c1C1=Nc2cn[nH]c2-c2cc(C(F)(F)F)cc3[nH]cc1c23]

### Step 1. 4-(difluoromethoxy)-2,6-difluorobenzaldehyde (409-a)

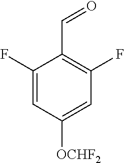 [SMILES: O=Cc1c(F)cc(OC(F)F)cc1F]

To a mixture of 2,6-difluoro-4-hydroxy-benzaldehyde (100 mg, 632 μmol) and (2-chloro-2,2-difluoroacetyl) oxysodium (193 mg, 1.27 mmol) in DMF (1.5 mL) and H₂O (0.15 mL) was added K₂CO₃ (105 mg, 759 μmol) and then heated to 100° C. for 2 hr. The reaction solution was ice-cooled, water (1.5 mL) and concentrated hydrochloric acid (0.6 mL) were added and the reaction solution was stirred at 25° C. for 16 hr. 2N sodium hydroxide aqueous solution was added to the reaction solution under ice-cooling to adjust the pH=10 and the mixture was extracted with ethyl acetate (10 mL×2). The organic layer was washed with brine (10 mL) and dried over Na₂SO₄. The solvent was removed under reduced pressure. The residue was purified by flash chromatography (ISCO®; 4 g SepaFlash® Silica Flash Column, petroleum ether/EtOAc with EtOAc from 0˜20%, 20 mL/min, 254 nm) to afford the title compound 409-a as a yellow solid (50 mg, 38%).

### Step 2. 409

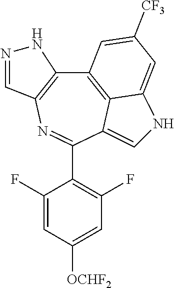 [SMILES: Fc1cc(OC(F)F)cc(F)c1C1=Nc2cn[nH]c2-c2cc(C(F)(F)F)cc3[nH]cc1c23]

The title compound was made from intermediate 401-c (Example 63, Step 3), following synthetic procedure similar as described for compound 401 (Example 63, Steps 4-5), except that 409-a was used in the place of 2,6-difluorobenzaldehyde. The crude product was purified by reverse phase preparative HPLC (column: Boston Green ODS 150×30 mm×5 μm; mobile phase: [water (HCl)-MeCN]; B %: 22%-62%, 9 min), to provide the title compound as orange solid (44.7 mg, 70.7%). MS (ESI) [M+H]⁺ m/z 455.0.

### Example 47. 9-(4-chloro-2,6-difluorophenyl)-3,6,7,8-tetrahydro-2,3,5,5a,8a,10-hexaazabenzo[cd]cyclopenta[h]azulene (Compound 385)

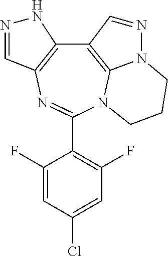 [SMILES: Fc1cc(Cl)cc(F)c1C1=Nc2cn[nH]c2-c2cnn3c2N1CCC3]

The title compound was made from intermediate 340-g (Example 4, Step 7), following synthetic procedure similar as described in the synthesis of compound 340 (Example 4, Steps 8-9), except that 4-chloro-2,6-difluorobenzaldehyde was used in the place of 2-fluoro-6-methyl-benzaldehyde. The crude product was purified by reverse phase preparative HPLC (column: 3_Phenomenex Luna C18 75×30 mm×3 μm; mobile phase: [water (0.225% FA)-MeCN]; B %: 20%-50%, 7.8 min), to provide the tile compound 385 as a yellow solid (15.8 mg, 12.0%). MS (ESI) [M+H]⁺ m/z 361.0.

### Example 48. 1-chloro-9-(3,5-difluoropyridin-4-yl)-3,6,7,8-tetrahydro-2,3,5,5a,8a,10-hexaazabenzo[cd]cyclopenta[h]azulene (Compound 386)

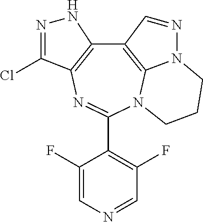 [SMILES: Fc1cncc(F)c1C1=Nc2c(Cl)n[nH]c2-c2cnn3c2N1CCC3]

The title compound was made from intermediate 340-b (Example 4, Step 2), following synthetic procedure similar as described in the synthesis of compound 376 (Example 39), except that 2-[(4-nitro-pyrazol-1-yl)methoxy]ethyl-trimethyl-silane was used in the place of 340-a. The crude product was purified by reverse phase preparative HPLC (column: Waters Xbridge BEH C18 150×25 mm×5 μm; mobile phase: [water (0.225% FA)-MeCN]; B %: 25%-55%, 9.5 min), to provide the title compound 386 as a yellow solid (0.69 mg, 6.4%). MS (ESI) [M+H]⁺ m/z 362.1.

### Example 49. 3-(2,4,6-trifluorophenyl)-2,7-dihydro-1H-2a,4,6,7,9,9a-hexaazadicyclopenta[cd,f]azulene (Compound 387)

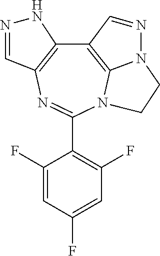 [SMILES: Fc1cc(F)c(C2=Nc3cn[nH]c3-c3cnn4c3N2CC4)c(F)c1]

The title compound was made from intermediate 373-a (Example 36, Step 1), following synthetic procedure similar as described in the synthesis of compound 340 (Example 4, Steps 6-9), except that 2,4,6-trifluorobenzaldehyde was used in the place of 2-fluoro-6-methyl-benzaldehyde. The crude product was purified by flash chromatography (ISCO®; 4 g SepaFlash® Silica Flash Column, petroleum ether/EtOAc with EtOAc from 0˜100%, 20 mL/min, 254 mn) and further purified by reverse phase preparative HPLC (column: 3_Phenomenex Luna C18 75×30 mm×3 μm; mobile phase: [water (FA)-MeCN]; B %: 5%-45%, 7.8 min), to provide the title compound 387 as a yellow solid (20.1 mg, 22.1%). MS (ESI) [M+H]⁺ m/z 331.0.

### Example 50. 3-(4-chloro-2,6-difluorophenyl)-2,7-dihydro-1H-2a,4,6,7,9,9a-hexaazadicyclopenta[cd,f]azulene (Compound 388)

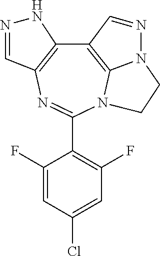 [SMILES: Fc1cc(Cl)cc(F)c1C1=Nc2cn[nH]c2-c2cnn3c2N1CC3]

The title compound was made from intermediate 373-a (Example 36, Step 1), following synthetic procedure similar as described in the synthesis of compound 340 (Example 4, Steps 6-9), except that 4-chloro-2,6-difluoro-benzaldehyde was used in the place of 2-fluoro-6-methyl-benzaldehyde. The crude product was purified by flash chromatography (ISCO®; 4 g SepaFlash® Silica Flash Column, petroleum ether/EtOAc with EtOAc from 0˜80%, 20 mL/min, 254 nm), to provide the title compound 388 as a yellow solid (8.34 mg, 22.5%). MS (ESI) [M+H]⁺ m/z 346.9.

### Example 51. 3-(3-bromo-2,6-difluorophenyl)-2,7-dihydro-1H-2a,4,6,7,9,9a-hexaazadicyclopenta[cd,f]azulene (Compound 389)

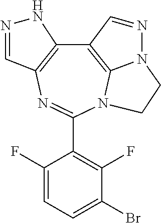 [SMILES: Fc1ccc(Br)c(F)c1C1=Nc2cn[nH]c2-c2cnn3c2N1CC3]

The title compound was made from intermediate 373-a (Example 36, Step 1), following synthetic procedure similar as described in the synthesis of compound 340 (Example 4, Steps 6-9), except that 3-bromo-2,6-difluoro-benzaldehyde was used in the place of 2-fluoro-6-methyl-benzaldehyde. The crude product was purified by flash chromatography (ISCO®; 4 g SepaFlash® Silica Flash Column, petroleum ether/EtOAc with EtOAc from 0˜80%, 20 mL/min, 254 nm), to provide the title compound 389 as a yellow solid (9.73 mg, 12.1%). MS (ESI) [M+H]⁺ m/z 391.0.

### Example 52. 3-(3-chloro-2,6-difluorophenyl)-2,7-dihydro-1H-2a,4,6,7,9,9a-hexaazadicyclopenta[cd,f]azulene (Compound 390)

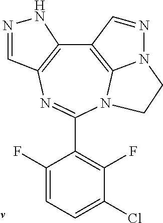 [SMILES: Fc1ccc(Cl)c(F)c1C1=Nc2cn[nH]c2-c2cnn3c2N1CC3]

The title compound was made from intermediate 373-a (Example 36, Step 1), following synthetic procedure similar as described in the synthesis of compound 340 (Example 4, Steps 6-9), except that 3-chloro-2,6-difluoro-benzaldehyde was used in the place of 2-fluoro-6-methyl-benzaldehyde. The crude product was purified by reverse phase preparative HPLC (column: Phenomenex C18 80×40 mm×3 μm; mobile phase: [water (NH4HCO3)-MeCN]; B %: 22%-52%, 7.8 min), to provide the title compound 390 as a yellow solid (24.6 mg, 11.7%). MS (ESI) [M+H]⁺ m/z 346.9.

### Example 53. 3-(3-bromo-2,6-difluorophenyl)-5-methyl-2,7-dihydro-1H-2a,4,6,7,9,9a-hexaazadicyclopenta[cd,f]azulene (Compound 391)

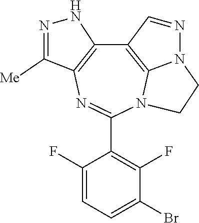 [SMILES: Cc1n[nH]c2c1N=C(c1c(F)ccc(Br)c1F)N1CCn3ncc-2c31]

The title compound was made from intermediate 372-b (Example 35, Step 2), following synthetic procedure similar as described in the synthesis of compound 340 (Example 4, Steps 6-9), except 3-bromo-2,6-difluoro-benzaldehyde was used in the place of 2-fluoro-6-methyl-benzaldehyde. The crude product was flash chromatography (ISCO®; 12 g SepaFlash® Silica Flash Column, petroleum ether/EtOAc with EtOAc from 0˜100%, 30 mL/min, 254 mn), to provide the title compound 391 as a yellow solid (12.7 mg, 11.4%). MS (ESI) [M+H]⁺ m/z 406.9.

### Example 54. 3-(3-bromo-2-chloro-6-fluorophenyl)-2,7-dihydro-1H-2a,4,6,7,9,9a-hexaazadicyclopenta[cd,f]azulene (Compound 392)

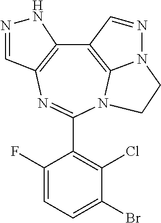 [SMILES: Fc1ccc(Br)c(Cl)c1C1=Nc2cn[nH]c2-c2cnn3c2N1CC3]

### Step 1. 3-bromo-2-chloro-6-fluorobenzaldehyde (392-a)

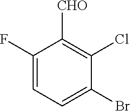 [SMILES: O=Cc1c(F)ccc(Br)c1Cl]

A solution of LDA/THF (2M, 11.9 mL, 23.9 mmol) was added to a mixture of 1-bromo-2-chloro-4-fluoro-benzene (5 g, 23.9 mmol) in THF (100 mL) at −78° C. The mixture was stirred at −78° C. for 30 minutes. Then DMF (1.84 mL, 23.9 mmol) was added and the mixture was stirred at −78° C. for another 30 minutes. The reaction mixture was diluted with H₂O (100 mL) and the mixture was extracted with EtOAc (100 mL×3). The combined organic phase was washed with brine (100 mL×2), dried with anhydrous Na₂SO₄, filtered and concentrated in vacuum. The residue was purified by column chromatography (petroleum ether/EtOAc=100/0, 254 nm) to afford title compound 392-a as a yellow solid (4 g, 70.6%).

### Step 2. 3-(3-bromo-2-chloro-6-fluorophenyl)-2,7-dihydro-1H-2a,4,6,7,9,9a-hexaazadicyclopenta[cd,f]azulene (Compound 392)

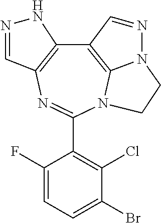 [SMILES: Fc1ccc(Br)c(Cl)c1C1=Nc2cn[nH]c2-c2cnn3c2N1CC3]

The title compound was made from intermediate 373-a (Example 36, Step 1), following synthetic procedure similar as described in the synthesis of compound 340 (Example 4, Steps 6-9), except that 392-a was used in the place of 2-fluoro-6-methyl-benzaldehyde. The crude product was purified by flash chromatography (ISCO®; 4 g SepaFlash® Silica Flash Column, petroleum ether/EtOAc with EtOAc from 0˜93%, 20 mL/min, 254 nm), to provide the title compound 392 as a yellow solid (21.2 mg, 38.2%). MS (ESI) [M+H]⁺ m/z 409.1.

### Example 55. 3-(2-bromo-6-fluorophenyl)-2,7-dihydro-1H-2a,4,6,7,9,9a-hexaazadicyclopenta[cd,f]azulene (Compound 393)

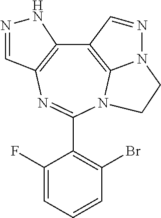 [SMILES: Fc1cccc(Br)c1C1=Nc2cn[nH]c2-c2cnn3c2N1CC3]

The title compound was made from intermediate 373-a (Example 36, Step 1), following synthetic procedure similar as described in the synthesis of compound 340 (Example 4, Steps 6-9), except that 2-bromo-6-fluoro-benzaldehyde was used in the place of 2-fluoro-6-methyl-benzaldehyde. The crude product was purified by flash chromatography (ISCO®; 4 g SepaFlash® Silica Flash Column, petroleum ether/EtOAc with EtOAc from 0˜100%, 25 mL/min, 254 mn), to provide the title compound 393 as a yellow solid (23.73 mg, 20.2%). MS (ESI) [M+H]⁺ m/z 375.0.

### Example 56. 3-(2,7-dihydro-1H-2a,4,6,7,9,9a-hexaazadicyclopenta[cd,f]azulen-3-yl)-2,4-difluorobenzonitrile (Compound 394)

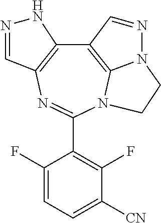 [SMILES: N#Cc1ccc(F)c(C2=Nc3cn[nH]c3-c3cnn4c3N2CC4)c1F]

The title compound was made from intermediate 373-a (Example 36, Step 1), following synthetic procedure similar as described in the synthesis of compound 340 (Example 4, Steps 6-9), except that 2,4-difluoro-3-formyl-benzonitrile was used in the place of 2-fluoro-6-methyl-benzaldehyde. The crude product was purified by flash chromatography (ISCO®; 4 g SepaFlash® Silica Flash Column, petroleum ether/EtOAc with EtOAc from 0˜80%, 20 mL/min, 254 nm), to provide the title compound 394 as a yellow solid (24.1 mg, 32.0%). MS (ESI) [M+H]⁺ m/z 337.9.

### Example 57. 3-(2-chloro-6-fluorophenyl)-5-methyl-2,7-dihydro-1H-2a,4,6,7,9,9a-hexaazadicyclopenta[cd,f]azulene (Compound 395)

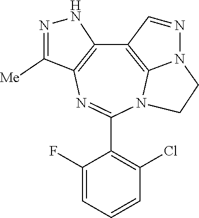 [SMILES: Cc1n[nH]c2c1N=C(c1c(F)cccc1Cl)N1CCn3ncc-2c31]

The title compound was made from intermediate 372-b (Example 35, Step 2), following synthetic procedure similar as described in the synthesis of compound 340 (Example 4, Steps 6-9), except that 2-chloro-6-fluoro-benzaldehyde was used in the place of 2-fluoro-6-methyl-benzaldehyde. The crude product was flash chromatography (ISCO®; 12 g SepaFlash® Silica Flash Column, petroleum ether/EtOAc with EtOAc from 0˜80%, 40 mL/min, 254 nm) and further purified by preparative TLC (silica, petroleum ether/EtOAc=0/1, 254 nm), to provide the title compound 395 as a yellow solid (21.75 mg, 8.5%). MS (ESI) [M+H]⁺ m/z 343.1.

### Example 58. 9-(2,6-difluoro-3-methylphenyl)-1-methyl-3,6,7,8-tetrahydro-2,3,5,5a,8a,10-hexaazabenzo[cd]cyclopenta[h]azulene (Compound 396)

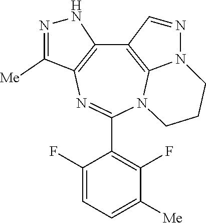 [SMILES: Cc1ccc(F)c(C2=Nc3c(C)n[nH]c3-c3cnn4c3N2CCC4)c1F]

The title compound was made from intermediate 340-b (Example 4, Step 2), following synthetic procedure similar as described in the synthesis of compound 340 (Example 4, Steps 3-9), except that 370-a was used in the place of 340-a and 2,6-difluoro-3-methyl-benzaldehyde was used in the place of 2-fluoro-6-methyl-benzaldehyde. The crude product was reverse phase preparative HPLC (column: Boston Green ODS 150×30 mm×5 μm; mobile phase: [water (HCl)-MECN]; B %: 17%-57%, 9 min), to provide the title compound 396 as a white solid (60 mg, 18.5%). MS (ESI) [M+H]⁺ m/z 355.1.

### Example 59 2,4-difluoro-3-(1-methyl-3,6,7,8-tetrahydro-2,3,5,5a,8a,10-hexaazabenzo[cd]cyclopenta[h]azulen-9-yl)benzonitrile (Compound 397)

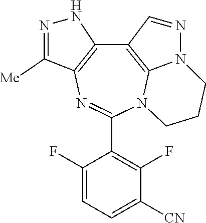 [SMILES: Cc1n[nH]c2c1N=C(c1c(F)ccc(C#N)c1F)N1CCCn3ncc-2c31]

The title compound was made from intermediate 340-b (Example 4, Step 2), following synthetic procedure similar as described in the synthesis of compound 340 (Example 4, Steps 3-9), except that 370-a was used in the place of 340-a and 2,4-difluoro-3-formyl-benzonitrile was used in the place of 2-fluoro-6-methyl-benzaldehyde. The crude product was flash chromatography (ISCO®; 4 g SepaFlash® Silica Flash Column, DCM/MeOH with MeOH from 0˜10%, 12 mL/min, 254 nm), to provide the title compound 397 as a white solid (28 mg, 22.3%). MS (ESI) [M+H]⁺ m/z 366.0.

### Example 60. 3-(4-(difluoromethoxy)-2,6-difluorophenyl)-5-methyl-2,7-dihydro-1H-2a,4,6,7,9,9a-hexaazadicyclopenta[cd,f]azulene (Compound 398)

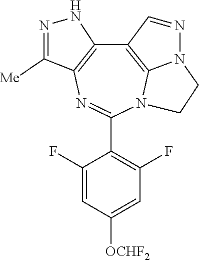 [SMILES: Cc1n[nH]c2c1N=C(c1c(F)cc(OC(F)F)cc1F)N1CCn3ncc-2c31]

### Step 1. 4-(difluoromethoxy)-2,6-difluorobenzaldehyde (398-a)

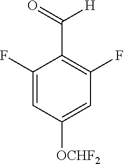 [SMILES: O=Cc1c(F)cc(OC(F)F)cc1F]

To a solution of 2,6-difluoro-4-hydroxy-benzaldehyde (500 mg, 3.16 mmol) in DMF (10 mL) was added K₂CO₃ (655.6 mg, 4.74 mmol) and sodium; 2-chloro-2,2-difluoro-acetate (964.3 mg, 6.33 mmol). The mixture was stirred at 100° C. for 2 hr. After addition, HCl/H₂O (768.7 mg, 6.33 mmol, 30 wt %) was added dropwise at 0° C. The resulting mixture was stirred at 25° C. for 16 hr. The reaction mixture was diluted with NaOH under ice-cooling to adjust the pH to 10, and extracted with EtOAc (50 mL×3). The combined organic layers were washed with brine (50 mL×2), dried with anhydrous Na₂SO₄, filtered and concentrated under reduced pressure. The residue was purified by flash chromatography (ISCO®; 40 g SepaFlash® Silica Flash Column, petroleum ether/EtOAc with EtOAc from 0˜5%, 30 mL/min, 254 nm) to give the title compound 398-a as a yellow oil (880 mg, 60.2%).

### Step 2. 3-(4-(difluoromethoxy)-2,6-difluorophenyl)-5-methyl-2,7-dihydro-1H-2a,4,6,7,9,9a-hexaazadicyclopenta[cd,f]azulene (Compound 398)

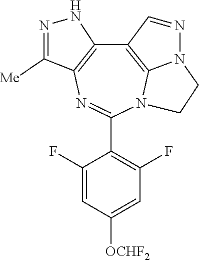 [SMILES: Cc1n[nH]c2c1N=C(c1c(F)cc(OC(F)F)cc1F)N1CCn3ncc-2c31]

The title compound was made from intermediate 372-b (Example 35, Step 2), following synthetic procedure similar as described in the synthesis of compound 340 (Example 4, Steps 6-9), except that 4-(difluoromethoxy)-2,6-difluoro-benzaldehyde was used in the place of 2-fluoro-6-methyl-benzaldehyde. The crude product was purified by flash chromatography (ISCO®; 12 g SepaFlash® Silica Flash Column, petroleum ether/EtOAc with EtOAc from 0˜100%, 50 mL/min, 254 nm), to provide the title compound 398 as a yellow solid (17.46 mg, 4.4%). MS (ESI) [M+H]+m/z 393.1.

### Example 61. 3-(3-cyclopropyl-2,6-difluorophenyl)-2,7-dihydro-1H-2a,4,6,7,9,9a-hexaazadicyclopenta[cd,f]azulene (Compound 399)

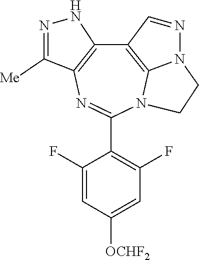 [SMILES: Cc1n[nH]c2c1N=C(c1c(F)cc(OC(F)F)cc1F)N1CCn3ncc-2c31]

### Step 1. 3-cyclopropyl-2,6-difluorobenzaldehyde (399-a)

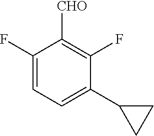 [SMILES: O=Cc1c(F)ccc(C2CC2)c1F]

To a solution of 3-bromo-2,6-difluoro-benzaldehyde (200 mg, 0.904 mmol) in toluene (4 mL) was added NaCO₃ (225.3 mg, 2.71 mmol), cyclopropylboronic acid (155.5 mg, 1.81 mmol) and palladium triphenylphosphane (31.4 mg, 0.027 mmol). The mixture was degassed and purged with nitrogen for three times. The mixture was stirred at 110° C. for 16 hr under nitrogen atmosphere. The mixture was diluted with H₂O (10 mL) and extracted with EtOAc (15 mL×3). The combined organic layers were washed with brine (10 mL), dried over Na₂SO₄, filtered and concentrated under reduced pressure. The residue was purified by flash chromatography (ISCO®; 20 g SepaFlash® Silica Flash Column, petroleum ether/EtOAc with EtOAc from 0˜10%, 30 mL/min, 254 nm), to provide the title compound 399-a as a colorless oil (65 mg, 39.4%).

### Step 2. 3-(3-cyclopropyl-2,6-difluorophenyl)-2,7-dihydro-1H-2a,4,6,7,9,9a-hexaazadicyclopenta[cd,f]azulene (Compound 399)

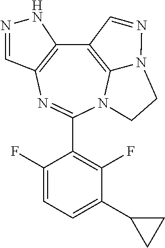 [SMILES: Fc1ccc(C2CC2)c(F)c1C1=Nc2cn[nH]c2-c2cnn3c2N1CC3]

The title compound was made from intermediate 373-a (Example 36, Step 1), following synthetic procedure similar as described in the synthesis of compound 340 (Example 4, Steps 6-9), except that 399-a was used in the place of 2-fluoro-6-methyl-benzaldehyde. The crude product was purified by reverse phase preparative HPLC (column: Waters Xbridge 150×25 mm×5 μm; mobile phase: [water (FA)-MeCN]; B %: 15%-55%, 7.8 min), to provide the title compound 399 as a yellow solid (8.38 mg, 10.0%). MS (ESI) [M+H]⁺ m/z 353.0.

### Example 62. 9-chloro-3-(2,6-difluorophenyl)-5-methyl-1,7-dihydropyrazolo[3′,4′:6,7]azepino[3,4,5-cd]indole (Compound 400)

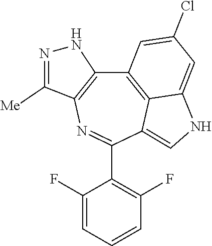 [SMILES: Cc1n[nH]c2c1N=C(c1c(F)cccc1F)c1c[nH]c3cc(Cl)cc-2c13]

### Step 1. 6-chloro-4-(3-methyl-4-nitro-1-((2-(trimethylsilyl)ethoxy)methyl)-1H-pyrazol-5-yl)-1H-indole (400-a)

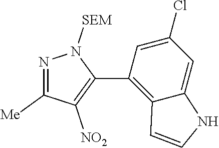 [SMILES: Cc1nn(COCC[Si](C)(C)C)c(-c2cc(Cl)cc3[nH]ccc23)c1[N+](=O)[O-]]

The title compound was made from 4-bromo-6-chloro-1H-indole, following synthetic procedure similar as described in the synthesis of compound 340-e (Example 4, Step 5), except that 3-methyl-4-nitro-1-((2-(trimethylsilyl)ethoxy)methyl)-1H-pyrazole was used in the place of 370-a, and that, 4-bromo-6-chloro-1H-indole was used in the place of 340-d. The crude product was purified by flash chromatography (ISCO®; 40 g SepaFlash® Silica Flash Column, petroleum ether/EtOAc with EtOAc from 0˜30%, 50 mL/min, 254 nm), to provide the title compound 400-a as a yellow oil (600 mg, 17.0%). MS (ESI) [M+H]⁺ m/z 407.1.

### Step 2. 9-chloro-3-(2,6-difluorophenyl)-5-methyl-1,7-dihydropyrazolo[3′,4′:6,7]azepino[3,4,5-cd]indole (Compound 400)

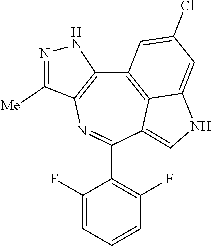 [SMILES: Cc1n[nH]c2c1N=C(c1c(F)cccc1F)c1c[nH]c3cc(Cl)cc-2c13]

The title compound was made from intermediate 400-a (Example 62, Step 1), following synthetic procedure similar as described in the synthesis of compound 375 (Example 38, Steps 2-4), except that 2,6-difluorobenzaldehyde was used in the place of 2-fluoro-6-methyl-benzaldehyde. The crude product was purified by flash chromatography (ISCO®; 4 g SepaFlash® Silica Flash Column, petroleum ether/EtOAc with EtOAc from 0˜100%, 30 mL/min, 254 nm) and further purified by reverse phase preparative HPLC (column: Boston Green ODS 150×30 mm×5 μm; mobile phase: [water (HCl)-MeCN]; B %: 17%-57%, 9 min), to provide the title compound 400 as a yellow solid (7.2 mg, 11.5%). MS (ESI) [M+H]⁺ m/z 369.0.

### Example 63. 3-(2,6-difluorophenyl)-9-(trifluoromethyl)-1,7-dihydropyrazolo[3′,4′:6,7]azepino[3,4,5-cd]indole (Compound 401)

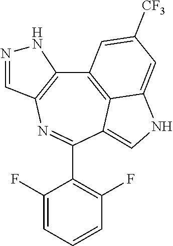 [SMILES: Fc1cccc(F)c1C1=Nc2cn[nH]c2-c2cc(C(F)(F)F)cc3[nH]cc1c23]

### Step 1. 4-(4,4,5,5-tetramethyl-1,3,2-dioxaborolan-2-yl)-6-(trifluoromethyl)-1H-indole (401-a)

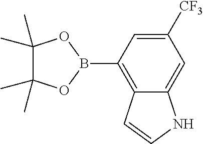 [SMILES: CC1(C)OB(c2cc(C(F)(F)F)cc3[nH]ccc23)OC1(C)C]

4-Bromo-6-(trifluoromethyl)-1H-indole (2.0 g, 7.57 mmol), 4,4,5,5-tetramethyl-2-(4,4,5,5-tetramethyl-1,3,2-dioxaborolan-2-yl)-1,3,2-dioxaborolane (2.31 g, 9.09 mmol), KOAc (2.23 g, 22.7 mmol) and Pd(dppf)Cl₂ (554 mg, 757 μmol) in dioxane (30 mL) was de-gassed and then heated to 90° C. for 3 hr under N₂. The mixture was filtered and concentrated. The residue was purified by flash chromatography (ISCO®; 25 g SepaFlash® Silica Flash Column, petroleum ether/EtOAc with EtOAc from 0˜30%, 100 mL/min, 254 nm) to afford the title compound 401-a (2.1 g, 89.1% yield) as a yellow solid. MS (ESI) [M+H]⁺ m/z 407.1.

### Step 2. 4-(1-(4-methoxybenzyl)-4-nitro-1H-pyrazol-5-yl)-6-(trifluoromethyl)-1H-indole (401-b)

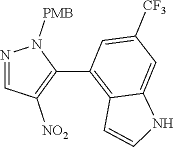

Intermediate 401-a (2.1 g, 6.75 mmol), 5-bromo-1-[(4-methoxyphenyl)methyl]-4-nitro-pyrazole (2.11 g, 6.75 mmol), K₂CO₃ (2.33 g, 16.9 mmol) and Pd(dppf)Cl₂ (494 mg, 675 μmol) in dioxane (40 mL) and H₂O (5 mL) was de-gassed and then heated to 90° C. for 3 hr under N₂. The mixture was filtered and concentrated. The residue was purified by flash chromatography (ISCO®; 25 g SepaFlash® Silica Flash Column, petroleum ether/EtOAc with EtOAc from 0˜30%, 100 mL/min, 254 nm) to afford the title compound 401-b as a yellow oil (2.0 g, 71.2%). MS (ESI) [M+H]⁺ m/z 417.1.

### Step 3. 1-(4-methoxybenzyl)-5-(6-(trifluoromethyl)-1H-indol-4-yl)-1H-pyrazol-4-amine (401-c)

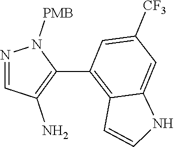

To a solution of 401-b (100 mg, 0.240 mmol) in MeOH (10 mL) under N₂ was added Pd/C (25.5 mg, 10 wt %). The suspension was degassed under vacuum and purged with hydrogen for several times. The mixture was stirred under hydrogen (30 psi) at 25° C. for 16 hr. The reaction mixture was filtered and washed with MeOH (10 mL) and concentrated in vacuum. The residue was purified by flash chromatography (ISCO®; 4 g SepaFlash® Silica Flash Column, petroleum ether/EtOAc with EtOAc from 0˜100%, 30 mL/min, 254 nm) to give the title compound 401-c as a yellow solid (35 mg, 37.7%). MS (ESI) [M+H]⁺ m/z 387.1.

### Step 4. 6-(2,6-difluorophenyl)-10-(4-methoxybenzyl)-2-(trifluoromethyl)-4,10-dihydropyrazolo[3′,4′:6,7]azepino[3,4,5-cd]indole (401-d)

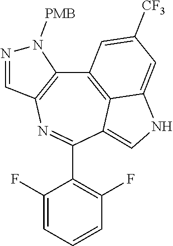

To a mixture of 401-c (35 mg, 90.6 μmol) in DCM (1 mL) at 20° C. under N2 was added 2,6-difluorobenzaldehyde (12.9 mg, 90.6 μmol) and TFA (10.3 mg, 90.6 μmol). The mixture was stirred at 25° C. for 30 minutes, I₂ (46 mg, 181.2 μmol) was added. This reaction was stirred for 16 hr. The reaction was quenched by NaHCO₃/H₂O (10 mL) and then extracted with DCM (10 mL×2). The combined organic phase was washed with brine (10 mL), dried over anhydrous Na₂SO₄, filtered and concentrated in vacuum. The residue was purified by flash chromatography (ISCO®; 4 g SepaFlash® Silica Flash Column, petroleum ether/EtOAc with EtOAc from 0˜100%, 30 mL/min, 254 nm) to give the title compound 401-d as a yellow solid (23 mg, 49.9%). MS (ESI) [M+H]⁺ m/z 509.1.

### Step 5. 3-(2,6-difluorophenyl)-9-(trifluoromethyl)-1,7-dihydropyrazolo[3′,4′:6,7]azepino[3,4,5-cd]indole (Compound 401)

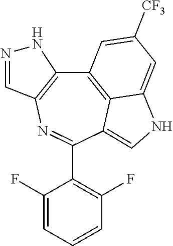 [SMILES: Fc1cccc(F)c1C1=Nc2cn[nH]c2-c2cc(C(F)(F)F)cc3[nH]cc1c23]

A mixture of 401-d (23 mg, 45.2 μmol) in TFA (2 mL) was stirred at 50° C. for 16 hr. The reaction mixture was concentrated under reduced pressure. The crude product was purified by reverse phase preparative HPLC (column: Boston Green ODS 150×30 mm×5 μm; mobile phase: [water (HCl)-MeCN]; B %: 16%-56%, 9 min), to provide the title compound 401 as a yellow solid (14 mg, 78.9%). MS (ESI) [M+H]⁺ m/z 389.2.

### Example 64. 9-(4-chloro-2,6-difluorophenyl)-3,6,7,8,9,10-hexahydro-2,3,5,5a,8a,10-hexaazabenzo[cd]cyclopenta[h]azulene (Compound 402)

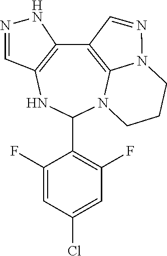 [SMILES: Fc1cc(Cl)cc(F)c1C1Nc2cn[nH]c2-c2cnn3c2N1CCC3]

A mixture of 340-g (100 mg, 489.6 μmol), 4-chloro-2,6-difluoro-benzaldehyde (86.5 mg, 0.490 mmol), K₂CO₃ (203.0 mg, 1.47 mmol) in t-BuOH (2 mL) and THE (1 mL) was stirred at 25° C. for 12 hr under N₂ atmosphere. The reaction mixture was concentrated under reduced pressure. The mixture was partitioned between DCM (10 mL×3) and brine (10 mL). The combined organic phases was washed with brine (10 mL), dried with anhydrous Na₂SO₄, filtered and concentrated in vacuum. The residue was purified by flash chromatography (ISCO®; 4 g SepaFlash® Silica Flash Column, petroleum ether/EtOAc with EtOAc from 0˜100%, 35 mL/min, 254 nm), to provide the title compound 402 as a yellow solid (132 mg, 64.5%). MS (ESI) [M+H]⁺ m/z 363.0.

### Example 65. 3-(3-bromo-2,6-difluorophenyl)-5-methyl-2,7-dihydro-1H-2a,4,6,7,9,9a-hexaazadicyclopenta[cd,f]azulene (Compound 403)

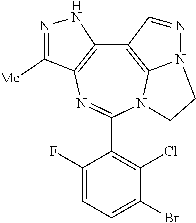 [SMILES: Cc1n[nH]c2c1N=C(c1c(F)ccc(Br)c1Cl)N1CCn3ncc-2c31]

The title compound was made from intermediate 372-b (Example 35, Step 2), following synthetic procedure similar as described in the synthesis of compound 340 (Example 4, Steps 6-9), except that 3-bromo-2-chloro-6-fluoro-benzaldehyde was used in the place of 2-fluoro-6-methyl-benzaldehyde. The crude product was purified by flash chromatography (ISCO®; 12 g SepaFlash® Silica Flash Column, petroleum ether/EtOAc with EtOAc from 0˜100%, 350 mL/min, 254 nm) and further purified by preparative TLC (silica, petroleum ether/EtOAc=0/1, 254 nm), to provide the title compound 403 as a yellow solid (28.54 mg, 6.8% yield). MS (ESI) [M+H]⁺ m/z 421.0.

### Example 66. 3-(5,7-difluoroquinolin-6-yl)-2,7-dihydro-1H-2a,4,6,7,9,9a-hexaazadicyclopenta[cd,f]azulene (Compound 404)

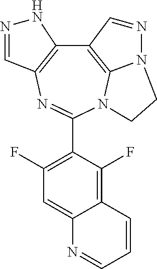 [SMILES: Fc1cc2ncccc2c(F)c1C1=Nc2cn[nH]c2-c2cnn3c2N1CC3]

### Step 1. 5,7-difluoroquinoline-6-carbaldehyde (404-a)

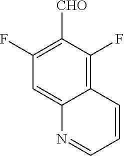 [SMILES: O=Cc1c(F)cc2ncccc2c1F]

To a −78° C. solution of 6-bromo-5,7-difluoro-quinoline (500 mg, 2.05 mmol) in THF (10 mL) was added n-BuLi (2.5 M, 0.82 mL). The mixture was stirred for at −78° C. for 30 in. A solution of DMF (149 mg, 2.05 mmol) in THF (2 mL) was added while the temperature was kept below −70° C., and the mixture was stirred at −78° C. for 30 minutes under N₂. The reaction mixture was warmed slowly to room temperature and diluted with aqueous saturated solution of NH₄Cl (10 ml.). The mixture was extracted with EtOAc (10 mL×3). The combined organic layers were washed with brine (20 mL), dried with anhydrous Na₂SO₄, filtered and concentrated under reduced pressure. The residue was purified by flash silica gel chromatography (ISCO®; 4 g SepaFlash® Silica Flash Column, petroleum ether/EtOAc with EtOAc from 0˜30%, 20 mL/min, 254 nm), to provide the title compound 404-a as a yellow solid (150 mg, 37.9%).

### Step 2. 3-(5,7-difluoroquinolin-6-yl)-2,7-dihydro-1H-2a,4,6,7,9,9a-hexaazadicyclopenta[cd,f]azulene (Compound 404)

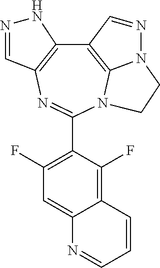 [SMILES: Fc1cc2ncccc2c(F)c1C1=Nc2cn[nH]c2-c2cnn3c2N1CC3]

The title compound was made from intermediate 373-a (Example 36, Step 1), following synthetic procedure similar as described in the synthesis of compound 340 (Example 4, Steps 6-9), except that 404-a was used in the place of 2-fluoro-6-methyl-benzaldehyde. The crude product was purified by flash chromatography (ISCO®; 4 g SepaFlash® Silica Flash Column, petroleum ether/EtOAc with EtOAc from 0-80%, 20 mL/min, 254 nm), to provide the title compound 404 as a yellow solid (38.1 mg, 37.9%). MS (ESI) [M+H]⁺ m/z 364.1.

### Example 67. 2,4-difluoro-3-(9-(trifluoromethyl)-1,7-dihydropyrazolo[3′,4′:6,7]azepino[3,4,5-cd]indol-3-yl)benzonitrile (Compound 405)

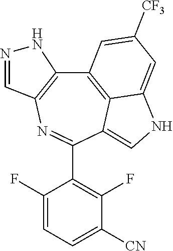 [SMILES: N#Cc1ccc(F)c(C2=Nc3cn[nH]c3-c3cc(C(F)(F)F)cc4[nH]cc2c34)c1F]

The title compound was made from intermediate 401-c (Example 63, Step 3), following synthetic procedure similar as described in the synthesis of compound 401 (Example 63, Steps 4-5), except that 2,4-difluoro-3-formylbenzonitrile was used in the place of 2,6-difluorobenzaldehyde. The crude product was purified by reverse phase preparative HPLC (column: Boston Green ODS 150×30 mm×5 μm; mobile phase: [water (HCl)-MeCN]; B %: 22%-62%, 9 min), to provide the title compound 405 as an orange solid (38.29 mg, 60.3%). MS (ESI) [M+H]⁺ m/z 414.0.

### Example 68. (3,5-difluoro-4-(9-(trifluoromethyl)-1,7-dihydropyrazolo[3′,4′:6,7]azepino[3,4,5-cd]indol-3-yl)phenyl)methanol (Compound 406)

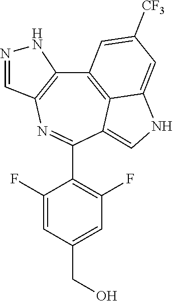 [SMILES: OCc1cc(F)c(C2=Nc3cn[nH]c3-c3cc(C(F)(F)F)cc4[nH]cc2c34)c(F)c1]

### Step 1. tert-butyl((3,5-difluorobenzyl)oxy)dimethylsilane (406-a)

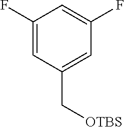 [SMILES: CC(C)(C)[Si](C)(C)OCc1cc(F)cc(F)c1]

To a mixture of (3,5-difluorophenyl)methanol (3.5 g, 24.3 mmol) in DMF (50 mL) was added imidazole (4.13 g, 60.7 mmol) and TBSCl (4.39 g, 29.1 mmol) at 10° C. under N₂. The mixture was stirred at 25° C. for 16 hr. The reaction was quenched by H₂O (60 mL) and then extracted with EtOAc (20 mL×2). The combined organic phase was washed with H₂O (20 mL×3), brine (20 mL), dried over anhydrous Na₂SO₄, filtered and concentrated in vacuo. The residue was purified by flash chromatography (ISCO®; 40 g SepaFlash® Silica Flash Column, petroleum ether/EtOAc with EtOAc from 0˜10%, 20 mL/min, 254 nm) to afford the title compound 406-a as a colorless oil (5.5 g, 87.6%).

### Step 2. 4-(((tert-butyldimethylsilyl)oxy)methyl)-2,6-difluorobenzaldehyde (406-b)

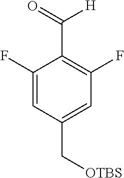 [SMILES: CC(C)(C)[Si](C)(C)OCc1cc(F)c(C=O)c(F)c1]

To a mixture of 406-a (5.5 g, 21.3 mmol) in THF (50 mL) was added dropwise LDA (2 M, 12.8 mL) at −30° C. under N₂. The mixture was stirred at −30° C. for 1 hour, DMF (1.87 g, 25.5 mmol) was added and stirred for 1 hour at −30° C. The reaction mixture was warmed to 0° C. and quenched by NH₄C1/H₂O (30 mL) slowly and then extracted with EtOAc (20 mL×2). The combined organic phase was washed with brine (20 mL), dried over anhydrous Na₂SO₄, filtered and concentrated in vacuo to give crude product. The residue was purified by flash chromatography (ISCO®; 40 g SepaFlash® Silica Flash Column, petroleum ether/EtOAc with EtOAc from 0-10%, 30 mL/min, 254 nm) to give the title compound 406-b as a colorless oil (4.6 g).

### Step 3. (3,5-difluoro-4-(9-(trifluoromethyl)-1,7-dihydropyrazolo[3′,4′:6,7]azepino[3,4,5-cd]indol-3-yl)phenyl)methanol (Compound 406)

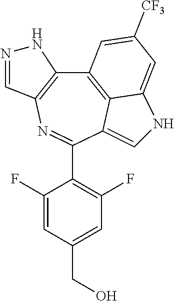 [SMILES: OCc1cc(F)c(C2=Nc3cn[nH]c3-c3cc(C(F)(F)F)cc4[nH]cc2c34)c(F)c1]

The title compound was made from intermediate 401-c (Example 63, Step 3), following synthetic procedure similar as described for compound 401 (Example 63, Steps 4-5), except that 406-b was used in the place of 2,6-difluorobenzaldehyde. The crude product was purified by reverse phase preparative HPLC (column: YMC-Triart Prep C18 150×40 mm×7 μm; mobile phase: [water (ammoniahydroxide v/v)-MeCN]; B %: 17%-57%, 9 min), to provide the title compound 406 as a yellow solid (19.2 mg, 46.2%). MS (ESI) [M+H]⁺ m/z 419.1.

### Example 69. (2,4-difluoro-3-(9-(trifluoromethyl)-1,7-dihydropyrazolo[3′,4′:6,7]azepino[3,4,5-cd]indol-3-yl)phenyl)methanol (Compound 407)

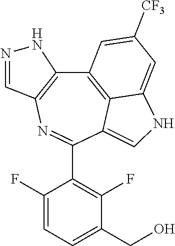 [SMILES: OCc1ccc(F)c(C2=Nc3cn[nH]c3-c3cc(C(F)(F)F)cc4[nH]cc2c34)c1F]

### Step 1. tert-butyl((2,4-difluorobenzyl)oxy)dimethylsilane (407-a)

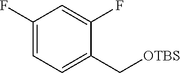 [SMILES: CC(C)(C)[Si](C)(C)OCc1ccc(F)cc1F]

To a mixture of (2,4-difluorophenyl)methanol (5 g, 34.7 mmol) in DMF (50 mL) was added imidazole (5.90 g, 86.7 mmol) and TBSCl (6.27 g, 41.6 mmol) at 10° C. under N₂. The mixture was stirred at 25° C. for 16 hr. The reaction was quenched by H₂O (60 mL) and then extracted with EtOAc (20 mL×2). The combined organic phase was washed with H₂O (20 mL×3), brine (20 mL), dried over anhydrous Na₂SO₄, filtered and concentrated in vacuo. The residue was purified by flash chromatography (ISCO®; 40 g SepaFlash® Silica Flash Column, petroleum ether/EtOAc with EtOAc from 0˜10%, 20 mL/min, 254 nm) to afford the title compound 407-a as a colorless oil (8.1 g, 90.4%).

### Step 2. 3-(((tert-butyldimethylsilyl)oxy)methyl)-2,6-difluorobenzaldehyde (407-b)

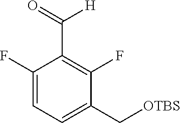 [SMILES: CC(C)(C)[Si](C)(C)OCc1ccc(F)c(C=O)c1F]

To a mixture of 407-a (1 g, 3.87 mmol) in THE (50 mL) was added dropwise LDA (2 M, 2.32 mL) at −30° C. under N₂. The mixture was stirred at −30° C. for 30 mins, DMF (339 mg, 4.64 mmol) was added and stirred for 1 hour at −30° C. The reaction mixture was warmed to 0° C. and quenched by NH₄Cl/H₂O (30 mL) slowly and then extracted with EtOAc (20 mL×2). The combined organic phase was washed with brine (20 mL), dried over anhydrous Na₂SO₄, filtered and concentrated in vacuo to give crude product. The residue was purified by flash chromatography (ISCO®; 40 g SepaFlash® Silica Flash Column, petroleum ether/EtOAc with EtOAc from 0˜10%, 30 mL/min, 254 nm) to give the title compound 407-b as a colorless oil (0.8 g, 72.2%).

### Step 3. (2,4-difluoro-3-(9-(trifluoromethyl)-1,7-dihydropyrazolo[3′,4′:6,7]azepino[3,4,5-cd]indol-3-yl)phenyl)methanol (Compound 407)

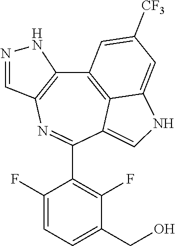 [SMILES: OCc1ccc(F)c(C2=Nc3cn[nH]c3-c3cc(C(F)(F)F)cc4[nH]cc2c34)c1F]

The title compound was made from intermediate 401-c (Example 63, Step 3), following synthetic procedure similar as described for compound 401 (Example 63, Steps 4-5), except that 407-b was used in the place of 2,6-difluorobenzaldehyde. The crude product was purified by reverse phase preparative HPLC (column: YMC-Triart Prep C18 150×40 mm×7 μm; mobile phase: [water (ammoniahydroxide v/v)-MeCN]; B %: 17%-57%, 9 min), to provide the title compound 407 as a yellow solid (22.2 mg, 57.7%). MS (ESI) [M+H]⁺ m/z 419.1.

### Example 70. 3-(2,6-difluoro-4-methoxyphenyl)-9-(trifluoromethyl)-1,7-dihydropyrazolo[3′,4′:6,7]azepino[3,4,5-cd]indole (Compound 408)

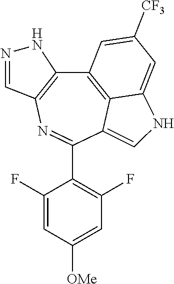 [SMILES: COc1cc(F)c(C2=Nc3cn[nH]c3-c3cc(C(F)(F)F)cc4[nH]cc2c34)c(F)c1]

The title compound was made from intermediate 401-c (Example 63, Step 3), following synthetic procedure similar as described for compound 401 (Example 63, Steps 4-5), except that 2,6-difluoro-4-methoxy-benzaldehyde was used in the place of 2,6-difluorobenzaldehyde. The crude product was purified by reverse phase preparative HPLC (column: Boston Green ODS 150×30 mm×5 μm; mobile phase: [water (HCl)-MeCN]; B %: 22%-62%, 9 min), to provide the title compound 408 as an orange solid (22.2 mg, 57.7%). MS (ESI) [M+H]⁺ m/z 419.0.

### Example 71: Kinase Inhibition

The compounds in Table 4 were tested for their inhibitory effect on LRRK2 kinases according to the following procedures.

Biochemical Assays:

The basic protocol for TR-FRET LanthaScreen Tb Kinase Activity Assay inhibitor studies were performed as follows. LanthaScreen Kinase Activity Assays (ThermoFisher/USA) to evaluate inhibitors were performed by addition of 100 nl of test compound in corresponding DMSO dilutions/5 μl of kinase/fluorescein-ERM(LRRKtide) peptide mixture, 5 μl of ATP into 384 well small volume plates. After incubation for 120 minutes at room temperature, the detection reagents containing Tb-anti-pLRRKtide antibody were added to monitor phosphrylation level of peptide. Then, after 60 min minutes incubation at room temperature plates were read in Envision. Data analysis of emission ratios was according to LanthaScreen Tb Kinase Activity Assay protocol.

Kinase and assay components were adjusted to final concentrations according to the kit protocol. For LRRK2: 2 nM wt human LRRK2, catalytic site, catalytic site (ThermoFisher/USA), 400 nM peptide, 38 uM ATP in 1× Kinase Buffer A.

Basic protocol for TR-FRET LanthaScreen Tb Kinase Activity Assay inhibitor studies involved two steps:

Enzymatic step: Addition of 100 nl of test compound in corresponding DMSO dilutions, 5 μl of kinase/substrate mixture, 5 μl of ATP into 384 well small volume plates. Incubation for 120 minutes at room temperature.

Detection step: Addition of 10 μl EDTA & antibody, read plate after 60 minutes. Data analysis of emission ratios according to KinEASE assay protocol.

Results for from biochemical tests were as follows:

### Inhibitory Activity Toward LRRK2 (Wt) and LRRK2 (G2019)

The following compounds had an IC₅₀ lower than 10 nM:
Compound 340; Compound 341; Compound 343; Compound 345; Compound 347; Compound 348; Compound 349; Compound 353; Compound 354; Compound 355; Compound 368; Compound 370; Compound 371; Compound 372; Compound 373; Compound 375; Compound 382; Compound 383; Compound 387; Compound 389; Compound 390; Compound 391; Compound 392; Compound 394; Compound 395; Compound 396; Compound 397; Compound 398; Compound 399; Compound 400; Compound 401; Compound 403; Compound 405; Compound 406; Compound 407;

The following compounds had an IC₅₀ between 10 nM and 100 nM:
Compound 342; Compound 344; Compound 346; Compound 350; Compound 351; Compound 356; Compound 357; Compound 358; Compound 359; Compound 360; Compound 361; Compound 362; Compound 367; Compound 376; Compound 380; Compound 381; Compound 384; Compound 385; Compound 386; Compound 388; Compound 393; Compound 404; Compound 408;

The following compounds had an IC₅₀ between 100 nM and 1000 nM:
Compound 363; Compound 364; Compound 366; Compound 374; Compound 377; Compound 379; Compound 402 Compound 369;

The following compounds had an IC₅₀ above 1000 nM:
Compound 352; Compound 365;

### Example 72: Kinase Inhibition

The compounds in Tables 5-6 were tested for their inhibitory effect on a set of kinases according to the following procedures.

TABLE 5

| Compound No. | IUPAC name |
|---|---|
| Compound 301 | 3-(2,6-difluorophenyl)-7H-1,2a,4,6,7-pentaazabenzo[cd]cyclopenta[f]azulene |
| Compound 302 | 4-(2,6-difluorophenyl)-8H-pyrazolo[4′,3′:4,5][1,3]diazepino[6,7,1-hi]indazole |
| Compound 303 | 3-(2,6-difluorophenyl)-7H-1,2a,4,6,7,9-hexaazabenzo[cd]cyclopenta[f]azulene |
| Compound 304 | 3-(2,6-difluorophenyl)-7H-1,2,2a,4,6,7-hexaazabenzo[cd]cyclopenta[f]azulene |
| Compound 305 | 3-(2,6-difluorophenyl)-1,7-dihydropyrazolo[3′,4′:6,7]azepino[3,4,5-cd]indole |
| Compound 306 | 4-(2-chloro-6-fluorophenyl)-8H-pyrazolo[4′,3′:4,5][1,3]diazepino[6,7,1-hi]indazole |
| Compound 307 | 9-(2,6-difluorophenyl)-3H-2,3,4,5,8a,10-hexaazabenzo[cd]cyclopenta[h]azulene |
| Compound 308 | 9-(2,6-difluorophenyl)-3,6,7,8-tetrahydro-2,3,5,5a,8a,10-hexaazabenzo[cd]cyclopenta[h]azulene |
| Compound 309 | 4-(2,6-dichlorophenyl)-8H-pyrazolo[4′,3′:4,5][1,3]diazepino[6,7,1-hi]indazole |
| Compound 310 | 3-(2,6-difluorophenyl)-1-methyl-1,7-dihydropyrazolo[3′,4′:6,7]azepino[3,4,5-cd]indole |
| Compound 311 | 1-(3-(2,6-difluorophenyl)pyrazolo[3′,4′:6,7]azepino[3,4,5-cd]indol-1(7H)-yl)ethan-1-one |
| Compound 312 | 3-(2,6-difluorophenyl)-1-isopropyl-1,7-dihydropyrazolo[3′,4′:6,7]azepino[3,4,5-cd]indole |
| Compound 313 | 10-(2,6-difluorophenyl)-7-methyl-3H-pyrazolo[4′,3′:4,5][1,3]diazepino[6,7,1-hi]indole |
| Compound 314 | 4-(2,6-difluorophenyl)-10-fluoro-7H-pyrazolo[4′,3′:4,5][1,3]diazepino[6,7,1-hi]indazole |
| Compound 315 | 4-(2,5-difluorophenyl)-8H-pyrazolo[4′,3′:4,5][1,3]diazepino[6,7,1-hi]indazole |
| Compound 316 | 4-(2-fluorophenyl)-8H-pyrazolo[4′,3′:4,5][1,3]diazepino[6,7,1-hi]indazole |
| Compound 317 | 4-(2,6-difluorophenyl)-11-fluoro-8H-pyrazolo[4′,3′:4,5][1,3]diazepino[6,7,1-hi]indazole |
| Compound 318 | 9-(2,4,6-trifluorophenyl)-3,6,7,8-tetrahydro-2,3,5,5a,8a,10-hexaazabenzo[cd]cyclopenta[h]azulene |
| Compound 319 | 3-(2,6-difluorophenyl)-2,7-dihydro-1H-2a,4,6,7,9,9a-hexaazadicyclopenta[cd,f]azulene |

TABLE 6

| | | HPLC- | retention | | | | | | |
|---|---|---|---|---|---|---|---|---|---|
| | | MS- | time | m + H | | LRRK2 | LRRK2 | | |
| Compound # | SMILES | Method | (min) | Found | NMR | wt | G2019S | TYK2 | NUAK1 |
| 301 | Fc1cccc(c1C1═Nc2c[nH]nc2—c2c3n1cnc3ccc2)F | HPLC- | 3.064 | 322.00 | 1H NMR (400 MHz, | A | A | B | B |
| | | MS B | | | DMSO-d₆) δ 7.85 (s, 1H), | | | | |
| | | | | | 7.81 (s, 1H), 7.69 (tt, J = | | | | |
| | | | | | 8.6, 6.7 Hz, 1H), 7.47 (d, J = | | | | |
| | | | | | 7.4 Hz, 1H), 7.41 (dd, J = | | | | |
| | | | | | 8.1, 1.1 Hz, 1H), 7.33 (t, J = | | | | |
| | | | | | 8.2 Hz, 2H), 7.18 (t, J = 7.8 | | | | |
| | | | | | Hz, 1H). | | | | |
| 302 | Fc1cccc(c1C1═Nc2c[nH]nc2—c2c3n1ncc3ccc2)F | HPLC- | 3.488 | 322.20 | 1H NMR (400 MHz, | A | A | B | A |
| | | MS B | | | DMSO-d₆) δ 8.11 (s, 1H), | | | | |
| | | | | | 7.85 (s, 1H), 7.63-7.51 (m, | | | | |
| | | | | | 2H), 7.48 (d, J = 8.0 Hz, | | | | |
| | | | | | 1H), 7.22 (t, J = 8.0 Hz, | | | | |
| | | | | | 2H), 7.15 (t, J = 7.7 Hz, | | | | |
| | | | | | 1H). | | | | |
| 303 | Fc1cccc(c1C1═Nc2cn[nH]c2—c2c3n1cnc3cnc2)F | HPLC- | 2.272 | 323.20 | — | A | A | C | B |
| | | MS B | | | | | | | |
| 304 | Fc1cccc(c1C1═Nc2cn[nH]c2—c2c3n1nnc3ccc2)F | HPLC- | 3.478 | 323.20 | — | B | B | D | B |
| | | MS B | | | | | | | |
| 305 | Fc1cccc(c1C1═Nc2cn[nH]c2—c2c3c1c[nH]c3ccc2)F | HPLC- | 2.342 | 321.20 | — | A | A | B | A |
| | | MS B | | | | | | | |
| 306 | Fc1cccc(c1C1═Nc2cn[nH]c2—c2c3n1ncc3ccc2)Cl | HPLC- | 3.596 | 338.00 | — | A | A | B | B |
| | | MS B | | | | | | | |
| 307 | Fc1cccc(c1C1═Nc2cn[nH]c2—c2c3—c(cccn13)nn2)F | HPLC- | 3.24 | 323.00 | — | B | C | D | C |
| | | MS B | | | | | | | |
| 308 | Fc1cccc(c1C1═Nc2cn[nH]c2—c2c3N1CCCn3nc2)F | HPLC- | 2.485 | 327.20 | 1H NMR (400 MHz, | A | A | B | B |
| | | MS B | | | Methanol-d₄) δ 7.65 (tt, J = | | | | |
| | | | | | 8.5, 6.5 Hz, 1H), 7.41 (s, | | | | |
| | | | | | 1H), 7.26-7.18 (m, 2H), | | | | |
| | | | | | 7.17 (s, 1H), 4.01 (t, J = 6.1 | | | | |
| | | | | | Hz, 2H), 3.47-3.40 (m, | | | | |
| | | | | | 2H), 2.08 (dq, J = 8.9, 5.9 | | | | |
| | | | | | Hz, 2H). | | | | |
| 309 | Clc1cccc(c1C1═Nc2c[nH]nc2—c2c3n1ncc3ccc2)Cl | HPLC- | 3.696 | 354.20 | — | B | B | B | C |
| | | MS B | | | | | | | |
| 310 | Fc1cccc(c1C1═Nc2cn[nH]c2—c2c3c1cn(c3ccc2)C)F | HPLC- | 3.36 | 335.20 | 1H NMR (400 MHz, | A | A | C | B |
| | | MS C | | | Methanol-d₄) δ 7.63 (tt, J = | | | | |
| | | | | | 8.5, 6.4 Hz, 1H), 7.56 (s, | | | | |
| | | | | | 1H), 7.39 (d, J = 6.6 Hz, | | | | |
| | | | | | 1H), 7.25-7.13 (m, 5H), | | | | |
| | | | | | 3.70 (s, 3H). | | | | |
| 311 | Fc1cccc(c1C1═Nc2c[nH]nc2—c2c3c1cn(c3ccc2)C(═O)C)F | HPLC- | 3.419 | 363.20 | — | A | A | C | B |
| | | MS B | | | | | | | |
| 312 | Fc1cccc(c1C1═Nc2c[nH]nc2—c2c3c1cn(c3ccc2)C(C)C)F | HPLC- | 2.924 | 363.20 | — | B | C | C | B |
| | | MS B | | | | | | | |
| 313 | Fc1cccc(c1C1═Nc2c[nH]nc2—c2c3n1cc(c3ccc2)C)F | HPLC- | 3.855 | 335.20 | — | B | B | D | B |
| | | MS B | | | | | | | |
| 314 | Fc1cc2cnn3c2c(—c2n[nH]cc2N═C3c2c(F)cccc2F)c1 | HPLC- | 3.726 | 340.20 | — | B | B | C | C |
| | | MS B | | | | | | | |
| 315 | Fc1ccc(c(c1)C1═Nc2c[nH]nc2—c2c3n1ncc3ccc2)F | HPLC- | 3.561 | 322.20 | — | B | B | C | B |
| | | MS B | | | | | | | |
| 316 | Fc1ccccc1C1═Nc2c[nH]nc2—c2c3n1ncc3ccc2 | HPLC- | 3.393 | 304.20 | — | A | B | C | B |
| | | MS B | | | | | | | |
| 317 | Fc1cccc(c1C1═Nc2cn[nH]c2—c2c3n1ncc3c(cc2)F)F | HPLC- | 3.744 | 340.20 | 1H NMR (400 MHz, | A | A | D | C |
| | | MS B | | | DMSO-d₆) δ 8.25 (s, 1H), | | | | |
| | | | | | 7.85 (s, 1H), 7.61-7.51 (m, | | | | |
| | | | | | 2H), 7.22 (t, J = 8.0 Hz, | | | | |
| | | | | | 2H), 6.99 (dd, J = 9.7, 8.1 | | | | |
| | | | | | Hz, 1H). | | | | |
| 318 | Fc1cc(F)c(c(c1)F)C1═Nc2cn[nH]c2—c2c3N1CCCn3nc2 | HPLC- | 2.673 | 345.20 | — | A | A | C | B |
| | | MS B | | | | | | | |
| 319 | Fc1cccc(c1C1═Nc2c[nH]nc2—c2c3N1CCn3nc2)F | HPLC- | 2.520 | 313.20 | 1H NMR (400 MHz, | A | A | B | C |
| | | MS C | | | Methanol-d₄) δ 7.54 (tt, J = | | | | |
| | | | | | 8.5, 6.5 Hz, 1H), 7.27 (s, | | | | |
| | | | | | 1H), 7.19-7.10 (m, 2H), | | | | |
| | | | | | 7.06 (s, 1H), 4.11 (dd, J = | | | | |
| | | | | | 9.1, 6.4 Hz, 2H), 3.91 (dd, J = | | | | |
| | | | | | 9.2, 6.4 Hz, 2H). | | | | |

Biochemical Assays:

The basic protocol for TR-FRET LanthaScreen Eu Kinase Binding Assay inhibitor studies were performed as follows. LanthaScreen Kinase Binding Assays (ThermoFisher/USA) to evaluate inhibitors were performed by addition of 5 μl of test compound in corresponding DMSO dilutions/5 μl of kinase/antibody mixture, 5 μl of tracer into 384 well small volume plates. After incubation for 1 hour at room temperature, plates were read. Data analysis of emission ratios was according to LanthaScreen Eu Kinase Binding Assay protocol.

Kinase and assay components were adjusted to final concentrations according to the kit protocol. For LRRK2: 5 nM wt human LRRK2, catalytic site, or G2019S human LRRK2, catalytic site (ThermoFisher/USA), 2 nM Eu-Anti-GST Antibody, 10 nM Kinase Tracer 236 in 1× Kinase Buffer A. For NUAK1: 8 nM wt human NUAK1, full length (ThermoFisher/USA), 2 nM Eu-Anti-His Antibody, 5 nM Kinase Tracer 236 in 1× Kinase Buffer A.

Basic protocol for HTRF KinEASE assay (Cisbio/FRA) inhibitor studies involved two steps:

Enzymatic step: Addition of 4 μl of test compound in corresponding DMSO dilutions, 4 μl of kinase/substrate mixture, 2 μl of ATP into 384 well small volume plates. Incubation for at least 30 minutes at room temperature.

Detection step: Addition of 5 μl antibody and 5 μl streptavidin-XL665, read plate after 60 minutes. Data analysis of emission ratios according to KinEASE assay protocol.

Kinase and assay components were adjusted to final concentrations according to the kit protocol. For TYK2: 2 nM wt human TYK2, catalytic site (SignalChem/CAN), 1 μM HTRF KinEASE-TK Substrate-biotin, 1 μM ATP in 1× Kinase Buffer

Results for from biochemical tests were as follows:

### Inhibitory Activity Toward LRRK2 (Wt) and LRRK2 (G2019)

The following compounds had an IC₅₀ lower than 10 nM: Compound 301; Compound 302; Compound 308; Compound 309; Compound 310; Compound 312; Compound 313; Compound 315; and Compound 317.

The following compounds had an IC₅₀ between 10 nM and 100 nM: Compound 304; Compound 305; Compound 306; Compound 307; Compound 311; and Compound 316.

The following compounds had an IC₅₀ between 100 nM and 1000 nM: Compound 303; and Compound 314.

### Inhibitory Activity Toward TYK2

The following compounds had an IC₅₀ lower than 10 nM: Compound 301; Compound 302; Compound 308; Compound 309; Compound 310; Compound 312; Compound 313; Compound 315; and Compound 317.

The following compounds had an IC₅₀ between 10 nM and 100 nM: Compound 304; Compound 305; Compound 306; Compound 307; Compound 308; Compound 311; and Compound 316.

The following compounds had an IC₅₀ between 100 nM and 1000 nM: Compound 303; and Compound 314.

### Inhibitory Activity Toward NUAK1

The following compounds had an IC₅₀ lower than 10 nM: Compound 309; and Compound 312.

The following compounds had an IC₅₀ between 10 nM and 100 nM: Compound 301; Compound 303; Compound 304; Compound 306; Compound 307; Compound 308; Compound 310; Compound 313; Compound 315; and Compound 317.

The following compounds had an IC₅₀ between 100 nM and 1000 nM: Compound 305; Compound 308; Compound 311; Compound 314; and Compound 316.

### INCORPORATION BY REFERENCE

References and citations to other documents, such as patents, patent applications, patent publications, journals, books, papers, web contents, have been made throughout this disclosure.

All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A compound of formula (I): 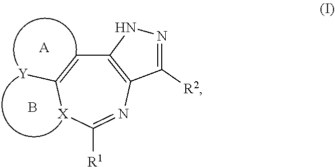
or a tautomer thereof, or a pharmaceutically acceptable salt thereof,
wherein:
R¹ is mono- or bicyclic aryl or heteroaryl;
R² is H, halo, OH, CN, CF₃, C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, or heteroaryl;
A is aryl, heteroaryl, cycloalkyl, heterocycloalkyl, cycloalkenyl, or heterocycloalkenyl;
B is aryl, heteroaryl, cycloalkyl, heterocycloalkyl, cycloalkenyl, or heterocycloalkenyl;
X is C or N; and
Y is C or N,
wherein each of the substituents in each of R¹, R², A, and B is independently optionally substituted with one or more moieties selected from the group consisting of halo, OH, CN, CF₃, NH₂, NO₂, C₁₋₆ alkyl, C₁₋₆ haloalkyl, C₁₋₆ thioalkyl, C₃₋₈ cycloalkyl, C₂₋₈ heterocycloalkyl, C₂₋₈ heterocycloalkenyl, C₂₋₆ alkenyl, C₂₋₆ alkynyl, C₁₋₆ alkoxy, C₁₋₆ haloalkoxy, C₁₋₆ alkylamino, C₂₋₆ dialkylamino, C₇₋₁₂ aralkyl, C₁₋₁₂ heteroaralkyl, aryl, heteroaryl, —C(O)R, —C(O)OR, —C(O)NRR′, —C(O)NRS(O)₂R′, —C(O)NRS(O)₂NR′R″, —OR, —OC(O)NRR′, —NRR′, —NRC(O)R′, —NRC(O)NR′R″, —NRS(O)₂R′, —NRS(O)₂NR′R″, —S(O)₂R, and —S(O)₂NRR′,
in which each of R, R′, and R″, independently, is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl, or R and R′, or R′ and R″, together with the nitrogen to which they are attached, form C₂₋₈ heterocycloalkyl.

2. The compound of claim 1, wherein A has 5 or 6 atoms.

3. The compound of claim 1, wherein B has 5 or 6 atoms.

4. The compound of claim 1, wherein R¹ is an optionally substituted 6-membered aryl or heteroaryl, or an optionally substituted 10-member bicyclic aryl or heteroaryl.

5. The compound of claim 4, wherein R¹ is substituted with 1-3 of a -Me, ester, halogen, alcohol, cyano, —CF₃, CHF₂, —OCHF₂, or cyclopropyl group.

6. The compound of claim 5, wherein R₁ has the structure: 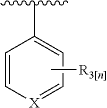
wherein X is carbon or nitrogen, and X may be in an ortho, meta, or para position on the ring,
where n is 0, 1, 2, or 3, and
wherein each R³ may be independently selected from a -Me, ester, halogen, alcohol, cyano, —CF₃, CHF₂, —OCHF₂, or cyclopropyl group.

7. The compound of claim 1, wherein R² is H.

8. The compound of claim 1, wherein X is N.

9. The compound of claim 1, wherein X is C.

10. The compound of claim 1, wherein the compound has a structure selected from the group consisting of Compounds 301-319 or 340-408 and tautomers thereof.

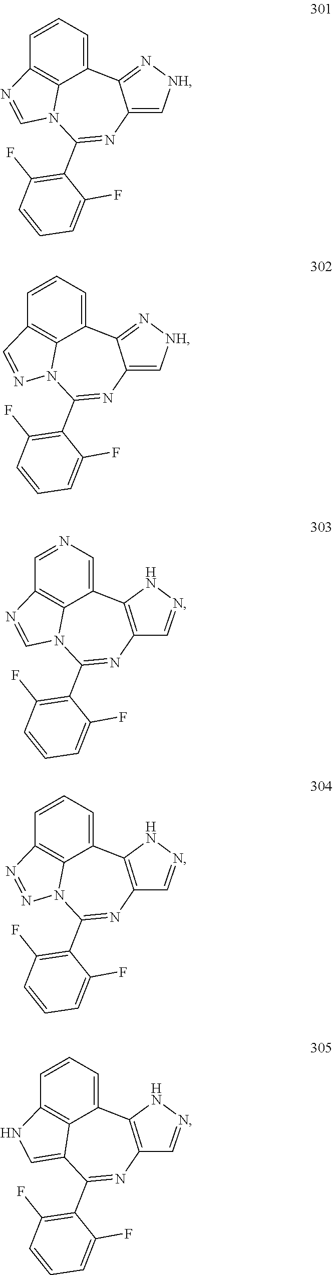

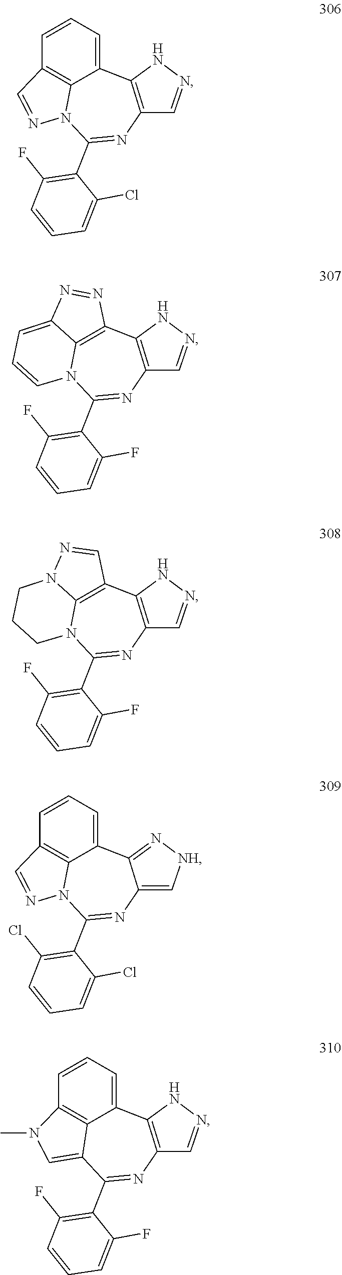

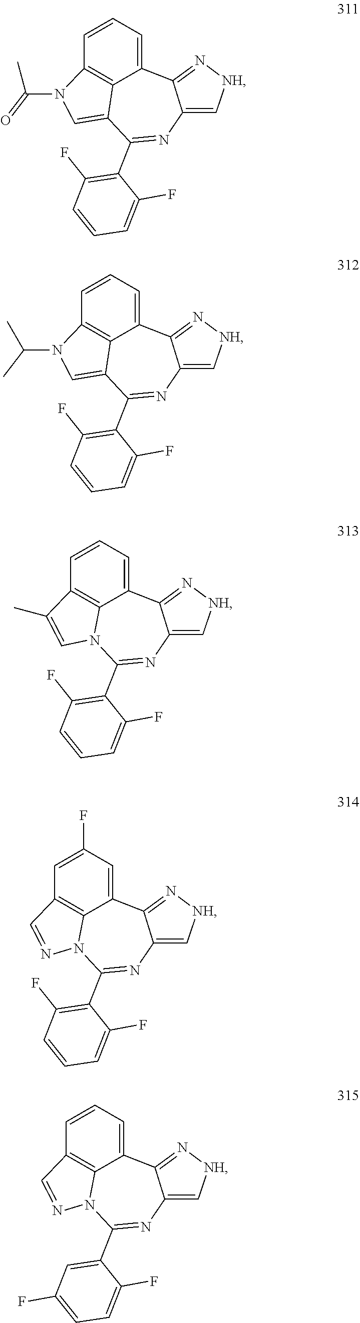

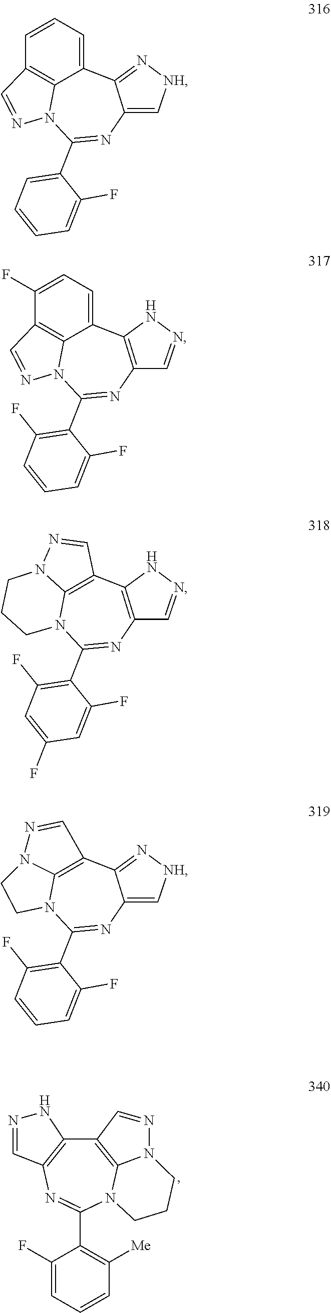

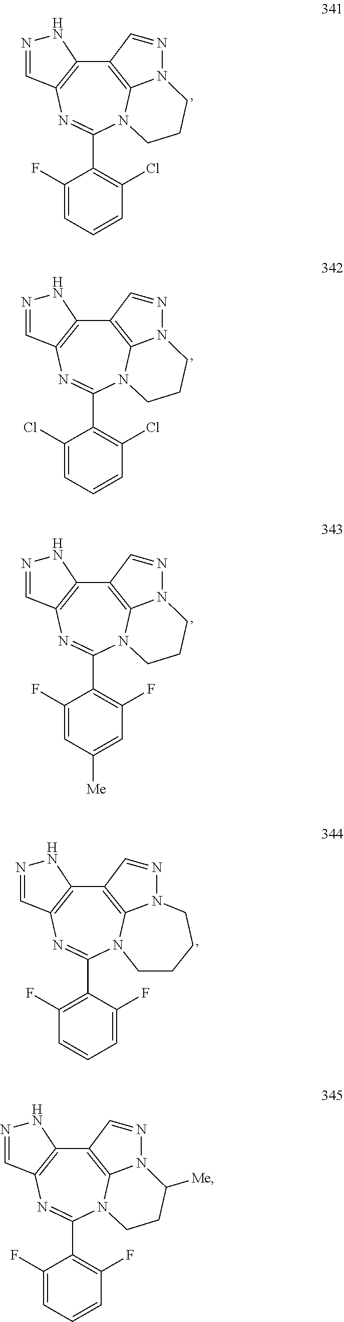 [SMILES: Fc1cccc(Cl)c1C1=Nc2cn[nH]c2-c2cnn3c2N1CCC3 ; Clc1cccc(Cl)c1C1=Nc2cn[nH]c2-c2cnn3c2N1CCC3 ; Cc1cc(F)c(C2=Nc3cn[nH]c3-c3cnn4c3N2CCC4)c(F)c1 ; Fc1cccc(F)c1C1=Nc2cn[nH]c2-c2cnn3c2N1CCCC3 ; CC1CCN2C(c3c(F)cccc3F)=Nc3cn[nH]c3-c3cnn1c32]

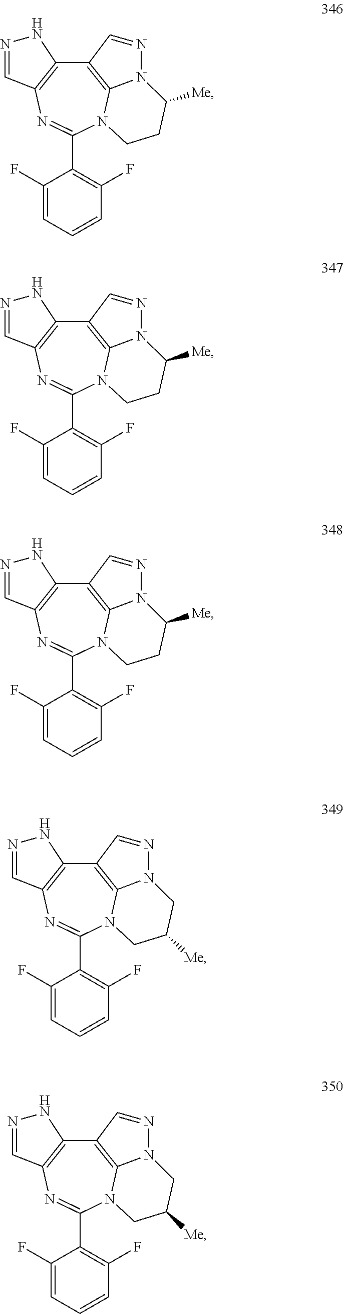 [SMILES: C[C@@H]1CCN2C(c3c(F)cccc3F)=Nc3cn[nH]c3-c3cnn1c32 ; C[C@H]1CCN2C(c3c(F)cccc3F)=Nc3cn[nH]c3-c3cnn1c32 ; C[C@H]1CCN2C(c3c(F)cccc3F)=Nc3cn[nH]c3-c3cnn1c32 ; C[C@@H]1CN2C(c3c(F)cccc3F)=Nc3cn[nH]c3-c3cnn(c32)C1 ; C[C@H]1CN2C(c3c(F)cccc3F)=Nc3cn[nH]c3-c3cnn(c32)C1]

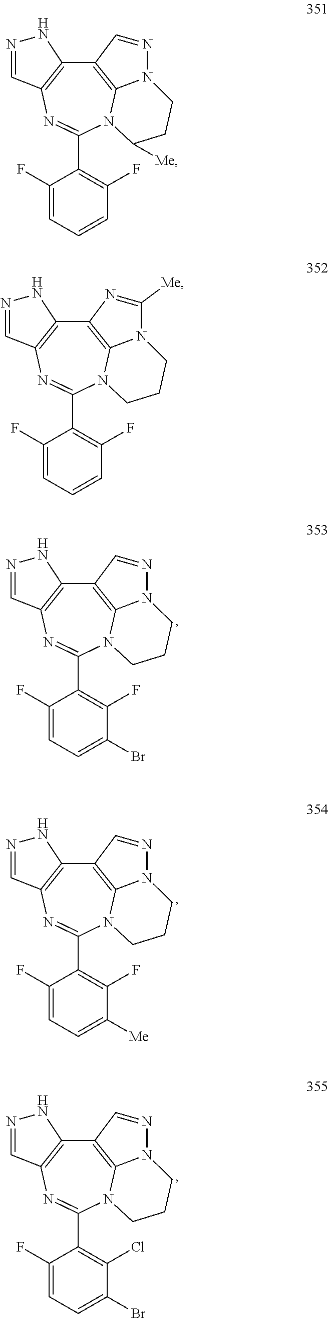 [SMILES: CC1CCn2ncc3c2N1C(c1c(F)cccc1F)=Nc1cn[nH]c1-3 ; Cc1nc2c3n1CCCN3C(c1c(F)cccc1F)=Nc1cn[nH]c1-2 ; Fc1ccc(Br)c(F)c1C1=Nc2cn[nH]c2-c2cnn3c2N1CCC3 ; Cc1ccc(F)c(C2=Nc3cn[nH]c3-c3cnn4c3N2CCC4)c1F ; Fc1ccc(Br)c(Cl)c1C1=Nc2cn[nH]c2-c2cnn3c2N1CCC3]

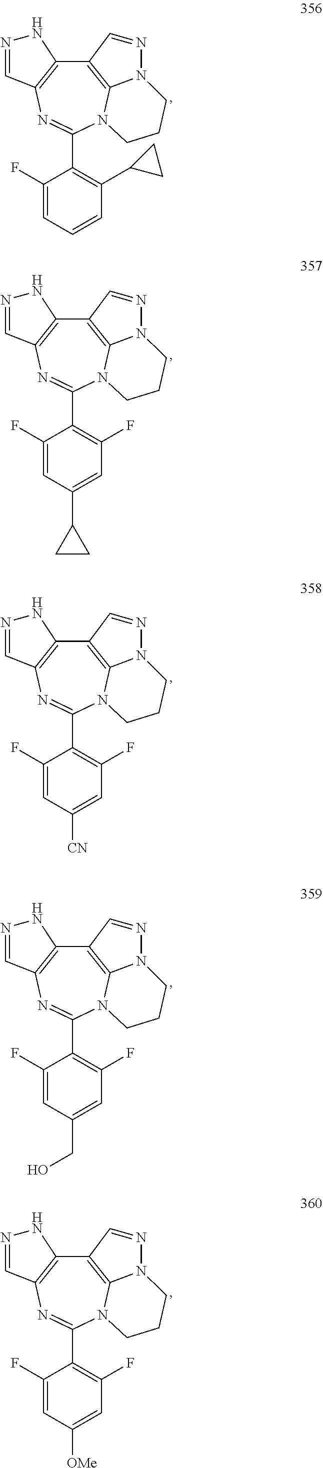 [SMILES: Fc1cccc(C2CC2)c1C1=Nc2cn[nH]c2-c2cnn3c2N1CCC3 ; Fc1cc(C2CC2)cc(F)c1C1=Nc2cn[nH]c2-c2cnn3c2N1CCC3 ; N#Cc1cc(F)c(C2=Nc3cn[nH]c3-c3cnn4c3N2CCC4)c(F)c1 ; OCc1cc(F)c(C2=Nc3cn[nH]c3-c3cnn4c3N2CCC4)c(F)c1 ; COc1cc(F)c(C2=Nc3cn[nH]c3-c3cnn4c3N2CCC4)c(F)c1]

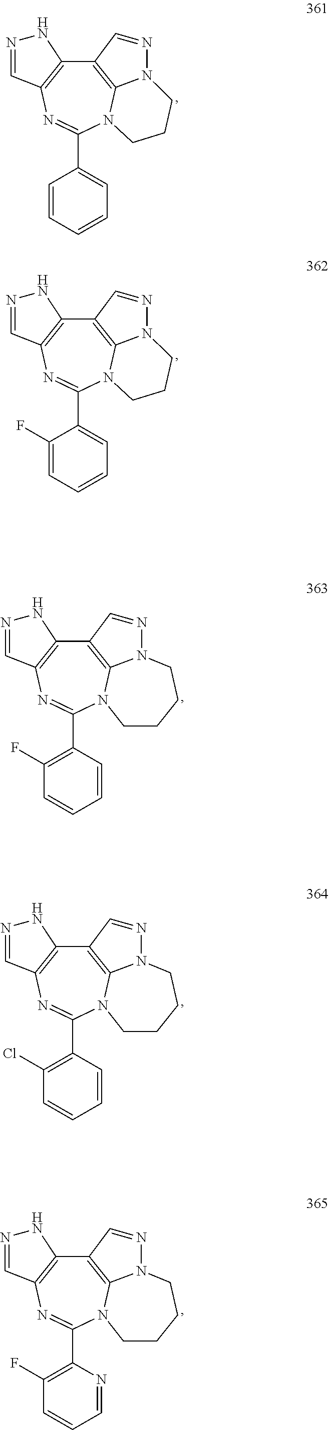 [SMILES: c1ccc(C2=Nc3cn[nH]c3-c3cnn4c3N2CCC4)cc1 ; Fc1ccccc1C1=Nc2cn[nH]c2-c2cnn3c2N1CCC3 ; Fc1ccccc1C1=Nc2cn[nH]c2-c2cnn3c2N1CCCC3 ; Clc1ccccc1C1=Nc2cn[nH]c2-c2cnn3c2N1CCCC3 ; Fc1cccnc1C1=Nc2cn[nH]c2-c2cnn3c2N1CCCC3]

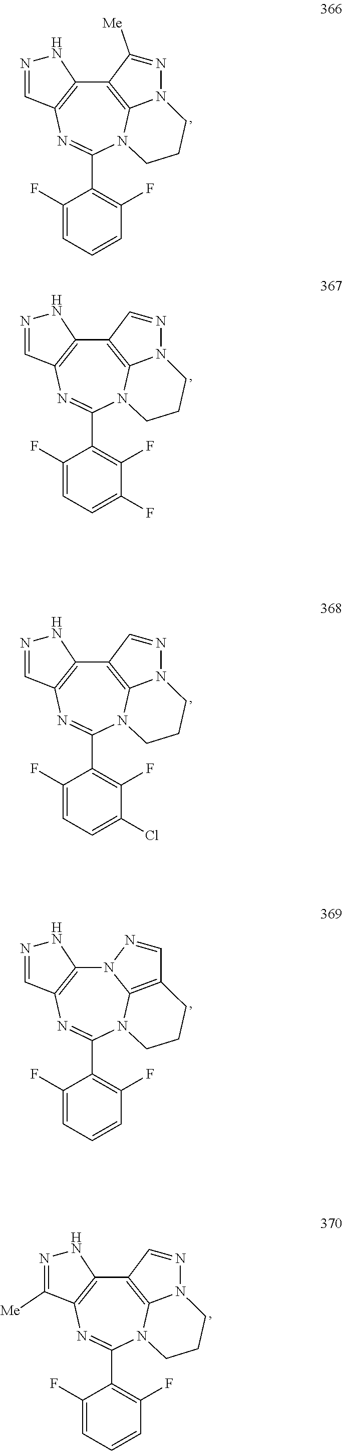 [SMILES: Cc1nn2c3c1-c1[nH]ncc1N=C(c1c(F)cccc1F)N3CCC2 ; Fc1ccc(F)c(C2=Nc3cn[nH]c3-c3cnn4c3N2CCC4)c1F ; Fc1ccc(Cl)c(F)c1C1=Nc2cn[nH]c2-c2cnn3c2N1CCC3 ; Fc1cccc(F)c1C1=Nc2cn[nH]c2-n2ncc3c2N1CCC3 ; Cc1n[nH]c2c1N=C(c1c(F)cccc1F)N1CCCn3ncc-2c31]

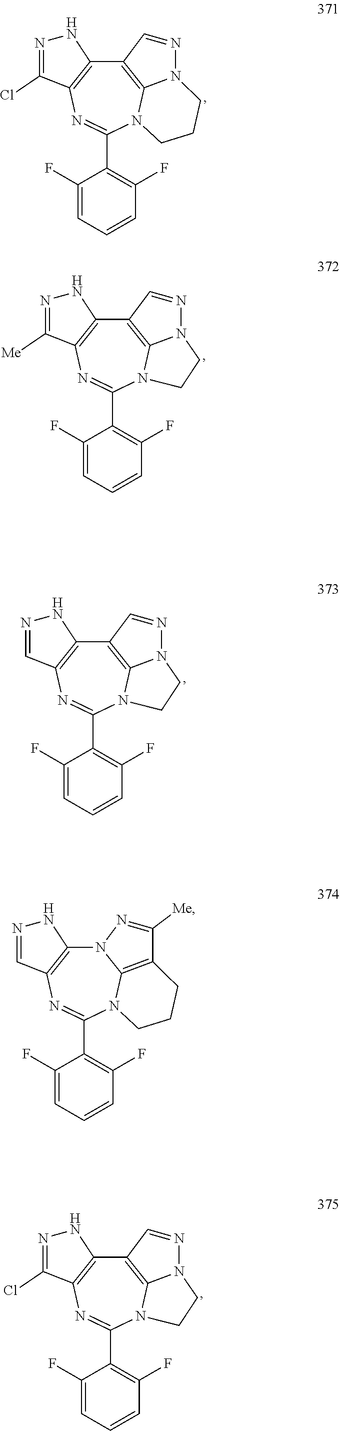 [SMILES: Fc1cccc(F)c1C1=Nc2c(Cl)n[nH]c2-c2cnn3c2N1CCC3 ; Cc1n[nH]c2c1N=C(c1c(F)cccc1F)N1CCn3ncc-2c31 ; Fc1cccc(F)c1C1=Nc2cn[nH]c2-c2cnn3c2N1CC3 ; Cc1nn2c3c1CCCN3C(c1c(F)cccc1F)=Nc1cn[nH]c1-2 ; Fc1cccc(F)c1C1=Nc2c(Cl)n[nH]c2-c2cnn3c2N1CC3]

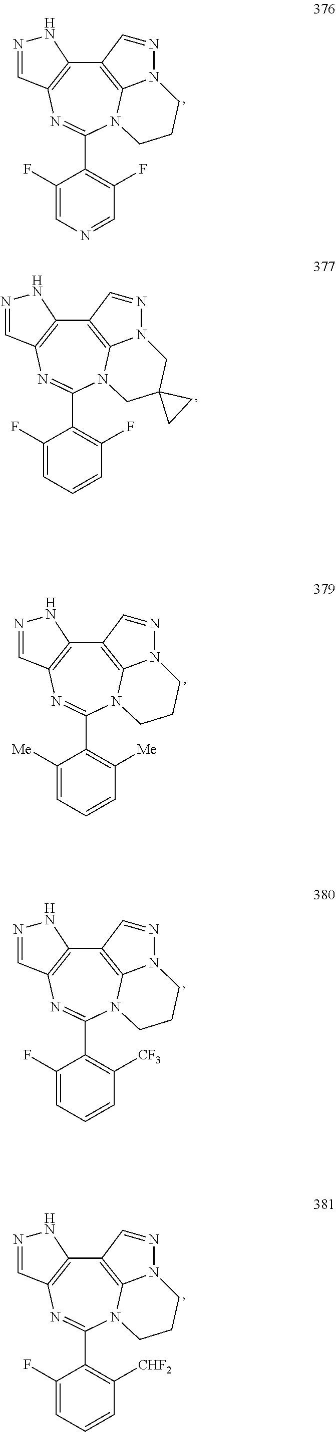 [SMILES: Fc1cncc(F)c1C1=Nc2cn[nH]c2-c2cnn3c2N1CCC3 ; Fc1cccc(F)c1C1=Nc2cn[nH]c2-c2cnn3c2N1CC1(CC1)C3 ; Cc1cccc(C)c1C1=Nc2cn[nH]c2-c2cnn3c2N1CCC3 ; Fc1cccc(C(F)(F)F)c1C1=Nc2cn[nH]c2-c2cnn3c2N1CCC3 ; Fc1cccc(C(F)F)c1C1=Nc2cn[nH]c2-c2cnn3c2N1CCC3]

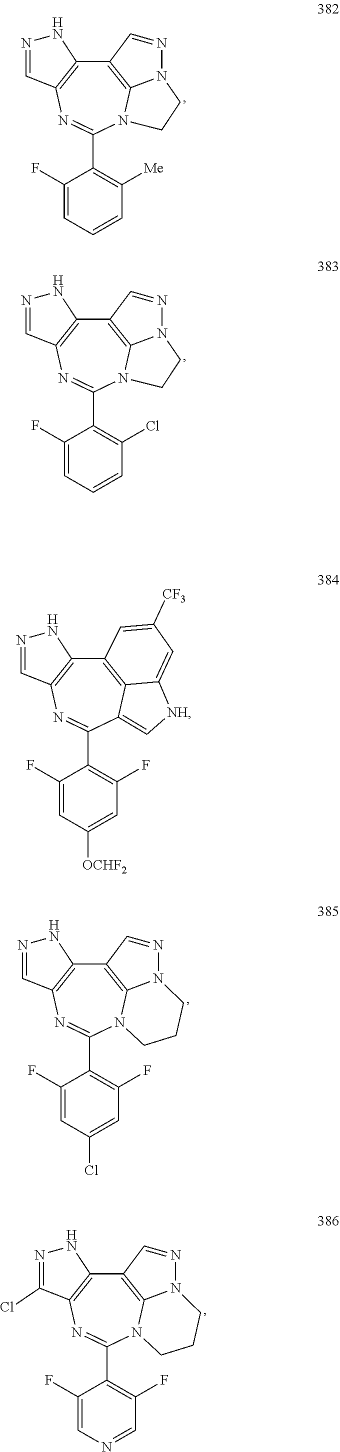 [SMILES: Cc1cccc(F)c1C1=Nc2cn[nH]c2-c2cnn3c2N1CC3 ; Fc1cccc(Cl)c1C1=Nc2cn[nH]c2-c2cnn3c2N1CC3 ; Fc1cc(OC(F)F)cc(F)c1C1=Nc2cn[nH]c2-c2cc(C(F)(F)F)cc3[nH]cc1c23 ; Fc1cc(Cl)cc(F)c1C1=Nc2cn[nH]c2-c2cnn3c2N1CCC3 ; Fc1cncc(F)c1C1=Nc2c(Cl)n[nH]c2-c2cnn3c2N1CCC3]

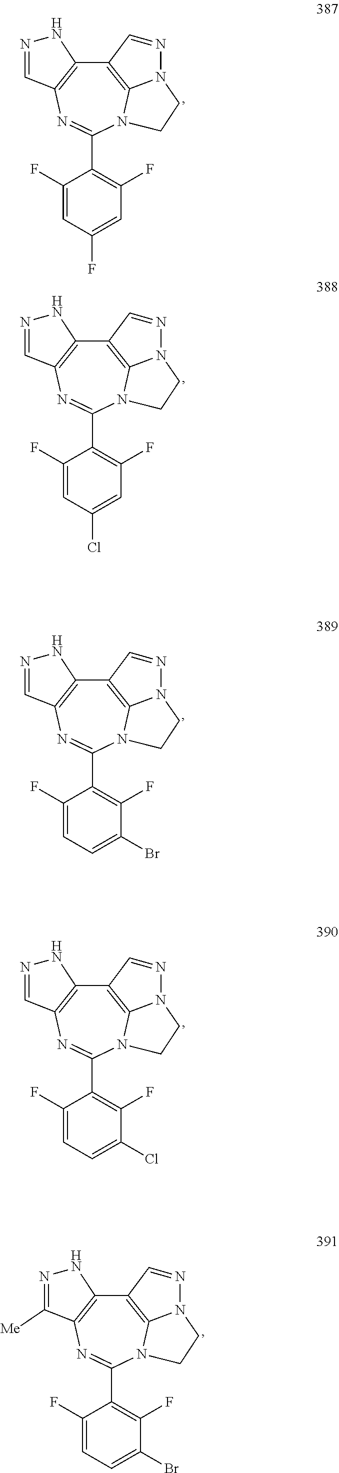 [SMILES: Fc1cc(F)c(C2=Nc3cn[nH]c3-c3cnn4c3N2CC4)c(F)c1 ; Fc1cc(Cl)cc(F)c1C1=Nc2cn[nH]c2-c2cnn3c2N1CC3 ; Fc1ccc(Br)c(F)c1C1=Nc2cn[nH]c2-c2cnn3c2N1CC3 ; Fc1ccc(Cl)c(F)c1C1=Nc2cn[nH]c2-c2cnn3c2N1CC3 ; Cc1n[nH]c2c1N=C(c1c(F)ccc(Br)c1F)N1CCn3ncc-2c31]

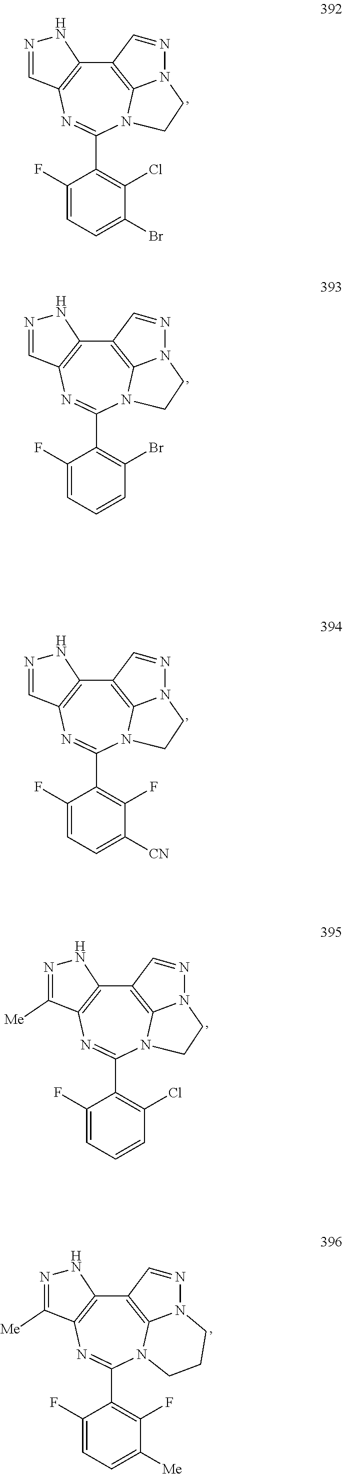 [SMILES: Fc1ccc(Br)c(Cl)c1C1=Nc2cn[nH]c2-c2cnn3c2N1CC3 ; Fc1cccc(Br)c1C1=Nc2cn[nH]c2-c2cnn3c2N1CC3 ; N#Cc1ccc(F)c(C2=Nc3cn[nH]c3-c3cnn4c3N2CC4)c1F ; Cc1n[nH]c2c1N=C(c1c(F)cccc1Cl)N1CCn3ncc-2c31 ; Cc1ccc(F)c(C2=Nc3c(C)n[nH]c3-c3cnn4c3N2CCC4)c1F]

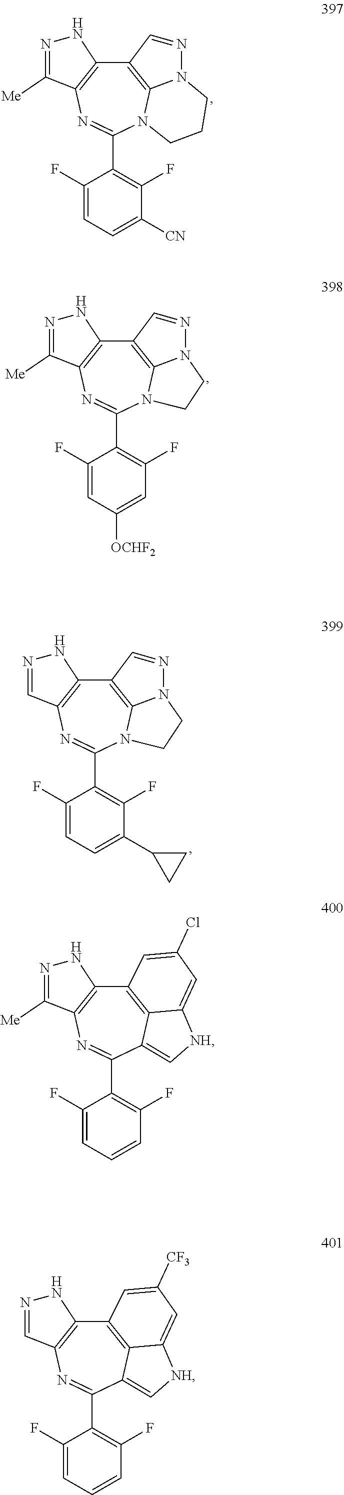

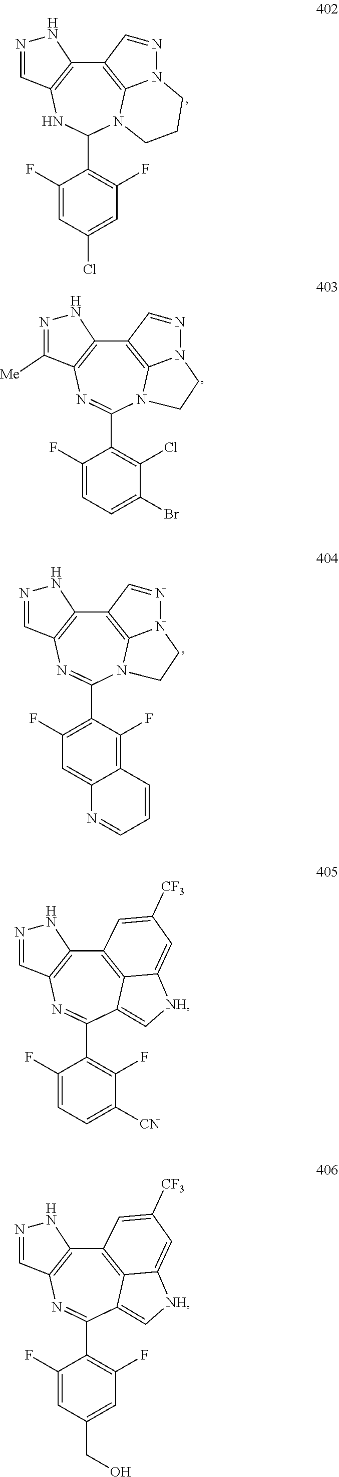

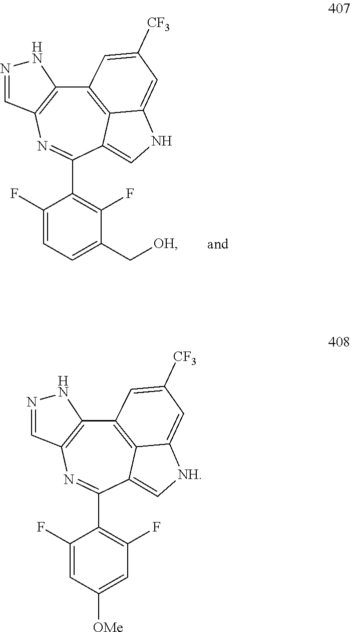 [SMILES: OCc1ccc(F)c(C2=Nc3cn[nH]c3-c3cc(C(F)(F)F)cc4[nH]cc2c34)c1F ; "and" ; COc1cc(F)c(C2=Nc3cn[nH]c3-c3cc(C(F)(F)F)cc4[nH]cc2c34)c(F)c1]

11-20. (canceled)

21. A pharmaceutical composition comprising a compound of formula (I): 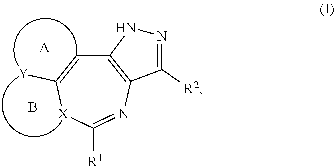
or a tautomer thereof, or a pharmaceutically acceptable salt thereof,
wherein:
R¹ is mono- or bicyclic aryl or heteroaryl;
R² is H, halo, OH, CN, CF₃, C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, or heteroaryl;
A is aryl, heteroaryl, cycloalkyl, heterocycloalkyl, cycloalkenyl, or heterocycloalkenyl;
B is aryl, heteroaryl, cycloalkyl, heterocycloalkyl, cycloalkenyl, or heterocycloalkenyl;
X is C or N; and
Y is C or N,
wherein each of the substituents in each of R¹, R², A, and B is independently optionally substituted with one or more moieties selected from the group consisting of halo, OH, CN, CF₃, NH₂, NO₂, C₁₋₆ alkyl, C₁₋₆ haloalkyl, C₁₋₆ thioalkyl, C₃₋₈ cycloalkyl, C₂₋₈ heterocycloalkyl, C₂₋₈ heterocycloalkenyl, C₂₋₆ alkenyl, C₂₋₆ alkynyl, C₁₋₆ alkoxy, C₁₋₆ haloalkoxy, C₁₋₆ alkylamino, C₂₋₆ dialkylamino, C₇₋₁₂ aralkyl, C₁₋₁₂ heteroaralkyl, aryl, heteroaryl, —C(O)R, —C(O)OR, —C(O)NRR′, —C(O)NRS(O)₂R′, —C(O)NRS(O)₂NR′R″, —OR, —OC(O)NRR′, —NRR′, —NRC(O)R′, —NRC(O)NR′R″, —NRS(O)₂R′, —NRS(O)₂NR′R″, —S(O)₂R, and —S(O)₂NRR′,
in which each of R, R′, and R″, independently, is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl, or R and R′, or R′ and R″, together with the nitrogen to which they are attached, form C₂₋₈ heterocycloalkyl.

22. The composition of claim 21, wherein A has 5 or 6 atoms.

23. The composition of claim 21, wherein B has 5 or 6 atoms.

24. The method of claim 21, wherein R¹ is an optionally substituted 6-membered aryl or heteroaryl or an optionally substituted 10-member bicyclic aryl or heteroaryl.

25. The composition of claim 24, wherein R¹ is substituted with 1-3 of a -Me, ester, halogen, alcohol, cyano, —CF₃, CHF₂, —OCHF₂, or cyclopropyl group.

26. The composition of claim 25, wherein R₁ has the structure: 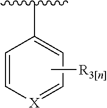
wherein X is carbon or nitrogen, and X may be in an ortho, meta, or para position on the ring,
where n is 0, 1, 2, or 3, and
wherein each R³ may be independently selected from a -Me, ester, halogen, alcohol, cyano, —CF₃, CHF₂, —OCHF₂, or cyclopropyl group.

27. The composition of claim 21, wherein R² is H.

28. The composition of claim 21, wherein X is N.

29. The composition of claim 21, wherein X is C.

30. The composition of claim 21, wherein the compound has a structure selected from the group consisting of Compounds 301-319 or 340-408 and tautomers thereof: 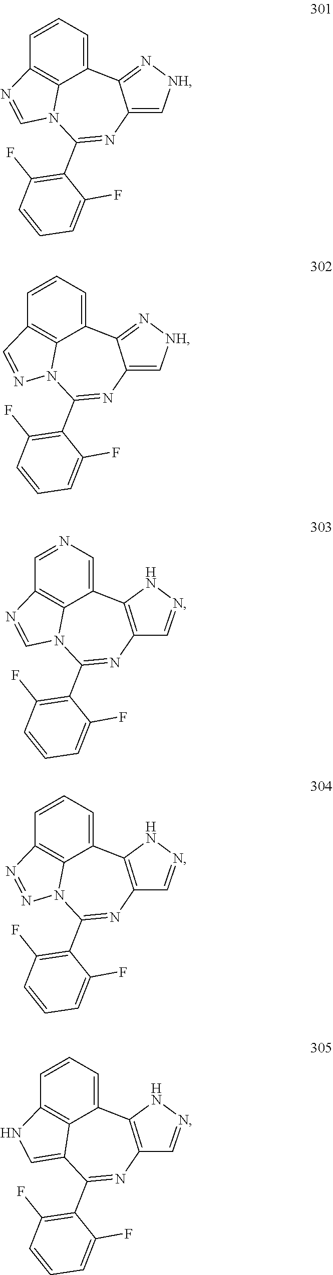 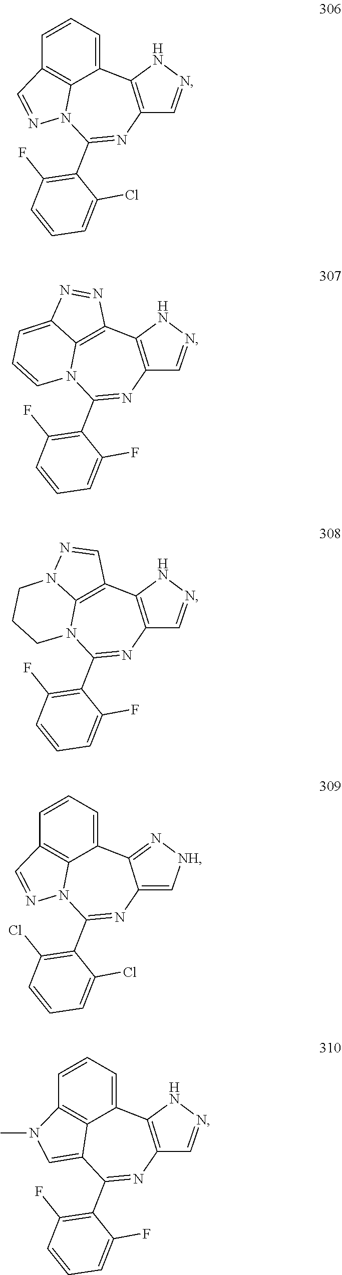 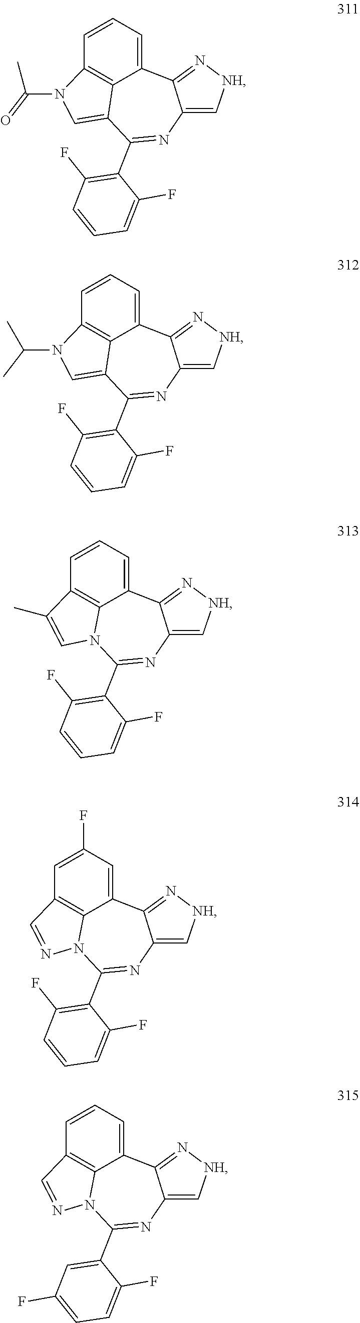 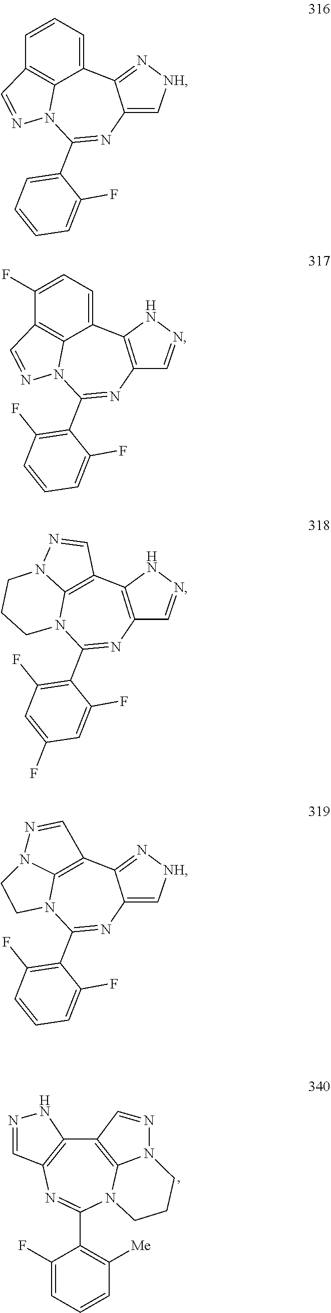 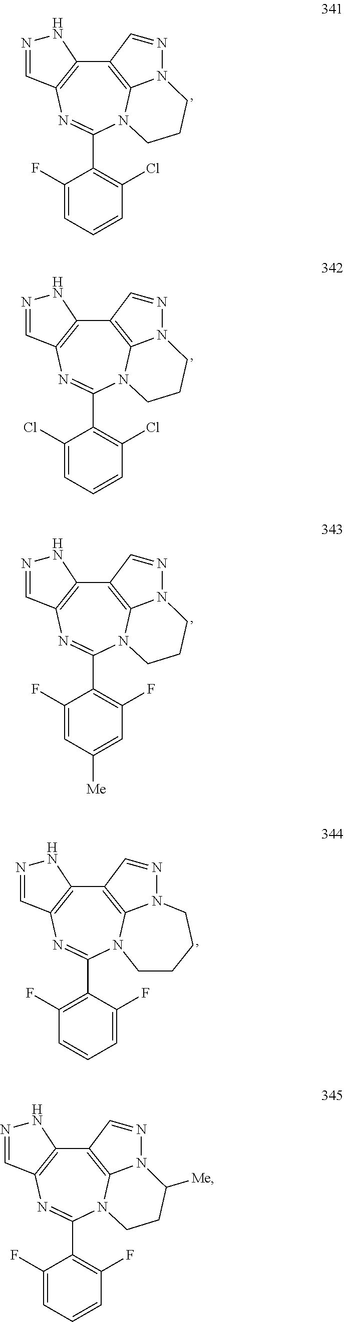 [SMILES: Fc1cccc(Cl)c1C1=Nc2cn[nH]c2-c2cnn3c2N1CCC3 ; Clc1cccc(Cl)c1C1=Nc2cn[nH]c2-c2cnn3c2N1CCC3 ; Cc1cc(F)c(C2=Nc3cn[nH]c3-c3cnn4c3N2CCC4)c(F)c1 ; Fc1cccc(F)c1C1=Nc2cn[nH]c2-c2cnn3c2N1CCCC3 ; CC1CCN2C(c3c(F)cccc3F)=Nc3cn[nH]c3-c3cnn1c32] 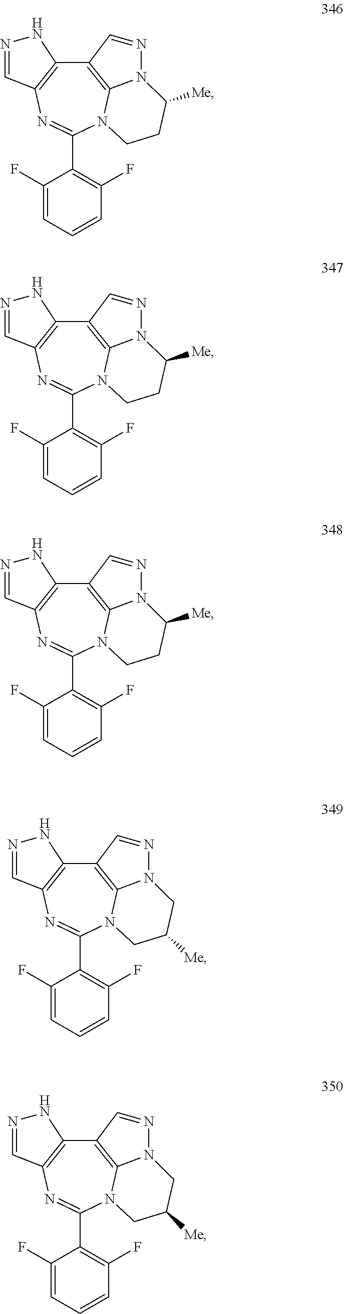 [SMILES: C[C@@H]1CCN2C(c3c(F)cccc3F)=Nc3cn[nH]c3-c3cnn1c32 ; C[C@H]1CCN2C(c3c(F)cccc3F)=Nc3cn[nH]c3-c3cnn1c32 ; C[C@H]1CCN2C(c3c(F)cccc3F)=Nc3cn[nH]c3-c3cnn1c32 ; C[C@@H]1CN2C(c3c(F)cccc3F)=Nc3cn[nH]c3-c3cnn(c32)C1 ; C[C@H]1CN2C(c3c(F)cccc3F)=Nc3cn[nH]c3-c3cnn(c32)C1] 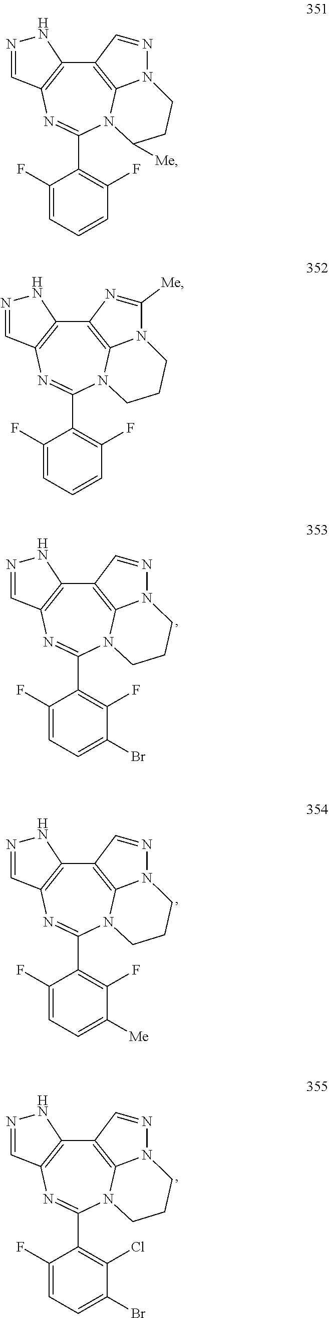 [SMILES: CC1CCn2ncc3c2N1C(c1c(F)cccc1F)=Nc1cn[nH]c1-3 ; Cc1nc2c3n1CCCN3C(c1c(F)cccc1F)=Nc1cn[nH]c1-2 ; Fc1ccc(Br)c(F)c1C1=Nc2cn[nH]c2-c2cnn3c2N1CCC3 ; Cc1ccc(F)c(C2=Nc3cn[nH]c3-c3cnn4c3N2CCC4)c1F ; Fc1ccc(Br)c(Cl)c1C1=Nc2cn[nH]c2-c2cnn3c2N1CCC3] 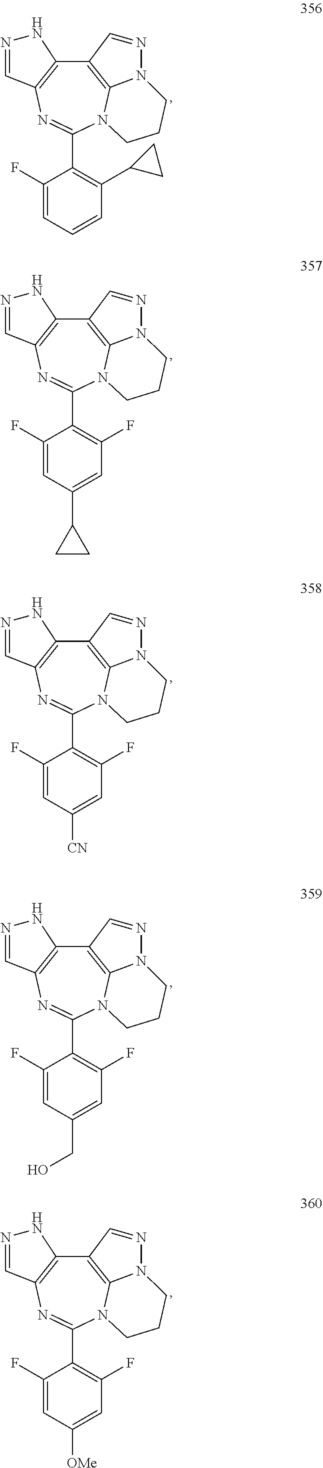 [SMILES: Fc1cccc(C2CC2)c1C1=Nc2cn[nH]c2-c2cnn3c2N1CCC3 ; Fc1cc(C2CC2)cc(F)c1C1=Nc2cn[nH]c2-c2cnn3c2N1CCC3 ; N#Cc1cc(F)c(C2=Nc3cn[nH]c3-c3cnn4c3N2CCC4)c(F)c1 ; OCc1cc(F)c(C2=Nc3cn[nH]c3-c3cnn4c3N2CCC4)c(F)c1 ; COc1cc(F)c(C2=Nc3cn[nH]c3-c3cnn4c3N2CCC4)c(F)c1] 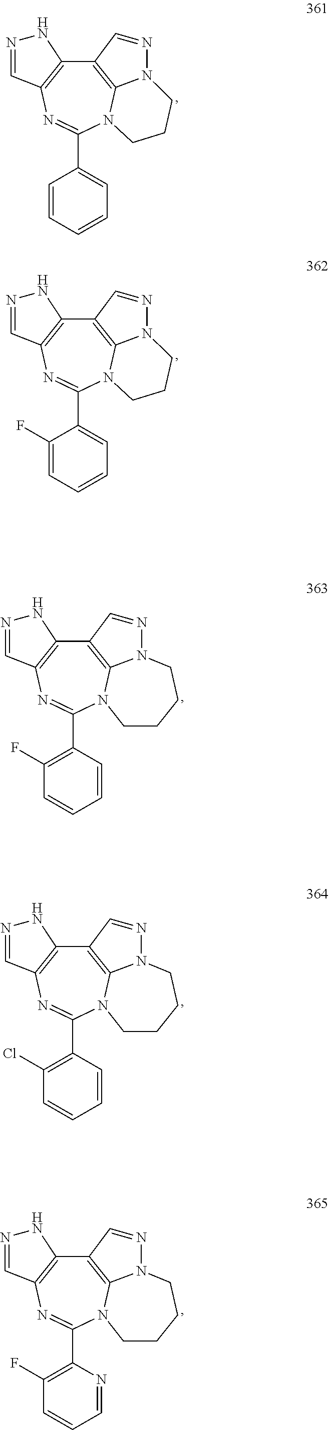 [SMILES: c1ccc(C2=Nc3cn[nH]c3-c3cnn4c3N2CCC4)cc1 ; Fc1ccccc1C1=Nc2cn[nH]c2-c2cnn3c2N1CCC3 ; Fc1ccccc1C1=Nc2cn[nH]c2-c2cnn3c2N1CCCC3 ; Clc1ccccc1C1=Nc2cn[nH]c2-c2cnn3c2N1CCCC3 ; Fc1cccnc1C1=Nc2cn[nH]c2-c2cnn3c2N1CCCC3] 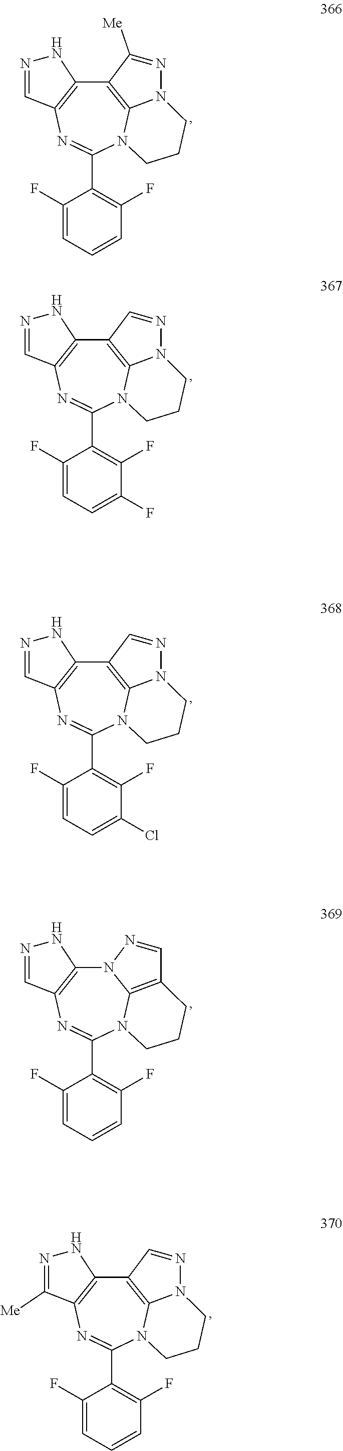 [SMILES: Cc1nn2c3c1-c1[nH]ncc1N=C(c1c(F)cccc1F)N3CCC2 ; Fc1ccc(F)c(C2=Nc3cn[nH]c3-c3cnn4c3N2CCC4)c1F ; Fc1ccc(Cl)c(F)c1C1=Nc2cn[nH]c2-c2cnn3c2N1CCC3 ; Fc1cccc(F)c1C1=Nc2cn[nH]c2-n2ncc3c2N1CCC3 ; Cc1n[nH]c2c1N=C(c1c(F)cccc1F)N1CCCn3ncc-2c31] 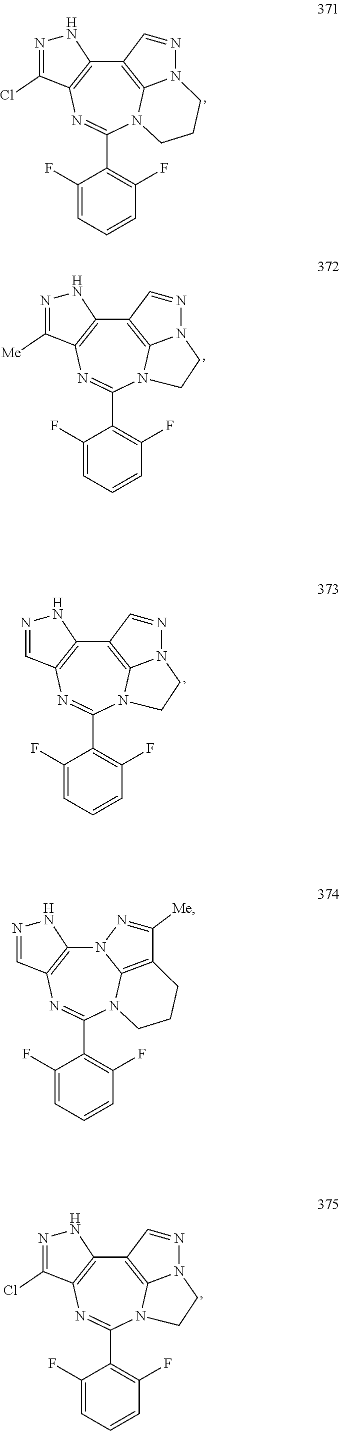 [SMILES: Fc1cccc(F)c1C1=Nc2c(Cl)n[nH]c2-c2cnn3c2N1CCC3 ; Cc1n[nH]c2c1N=C(c1c(F)cccc1F)N1CCn3ncc-2c31 ; Fc1cccc(F)c1C1=Nc2cn[nH]c2-c2cnn3c2N1CC3 ; Cc1nn2c3c1CCCN3C(c1c(F)cccc1F)=Nc1cn[nH]c1-2 ; Fc1cccc(F)c1C1=Nc2c(Cl)n[nH]c2-c2cnn3c2N1CC3] 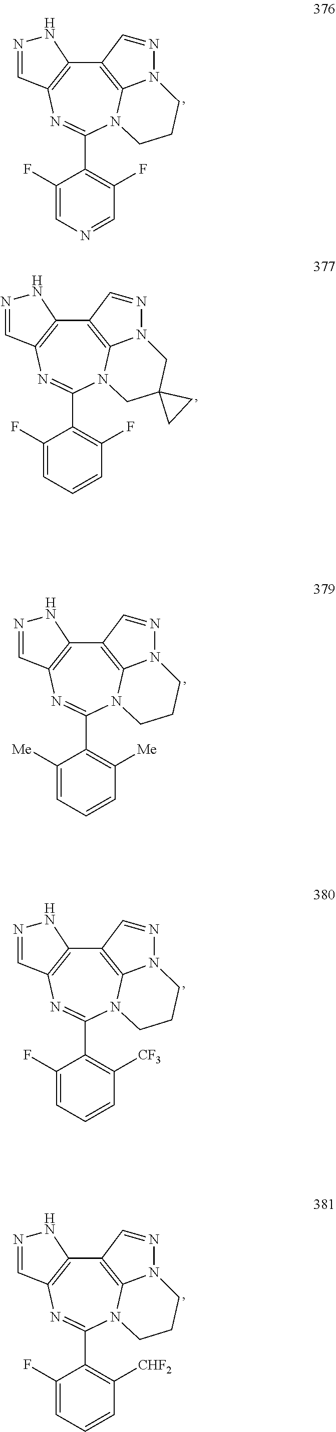 [SMILES: Fc1cncc(F)c1C1=Nc2cn[nH]c2-c2cnn3c2N1CCC3 ; Fc1cccc(F)c1C1=Nc2cn[nH]c2-c2cnn3c2N1CC1(CC1)C3 ; Cc1cccc(C)c1C1=Nc2cn[nH]c2-c2cnn3c2N1CCC3 ; Fc1cccc(C(F)(F)F)c1C1=Nc2cn[nH]c2-c2cnn3c2N1CCC3 ; Fc1cccc(C(F)F)c1C1=Nc2cn[nH]c2-c2cnn3c2N1CCC3] 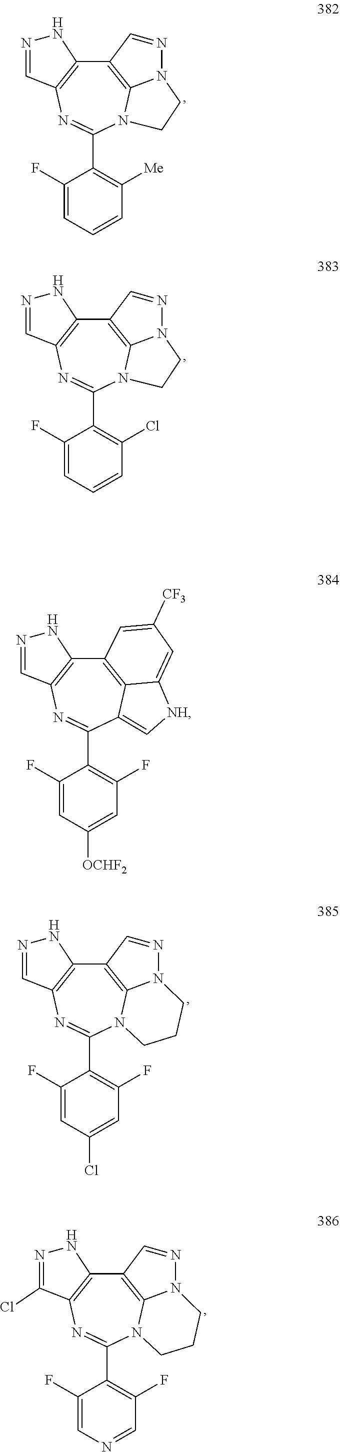 [SMILES: Cc1cccc(F)c1C1=Nc2cn[nH]c2-c2cnn3c2N1CC3 ; Fc1cccc(Cl)c1C1=Nc2cn[nH]c2-c2cnn3c2N1CC3 ; Fc1cc(OC(F)F)cc(F)c1C1=Nc2cn[nH]c2-c2cc(C(F)(F)F)cc3[nH]cc1c23 ; Fc1cc(Cl)cc(F)c1C1=Nc2cn[nH]c2-c2cnn3c2N1CCC3 ; Fc1cncc(F)c1C1=Nc2c(Cl)n[nH]c2-c2cnn3c2N1CCC3] 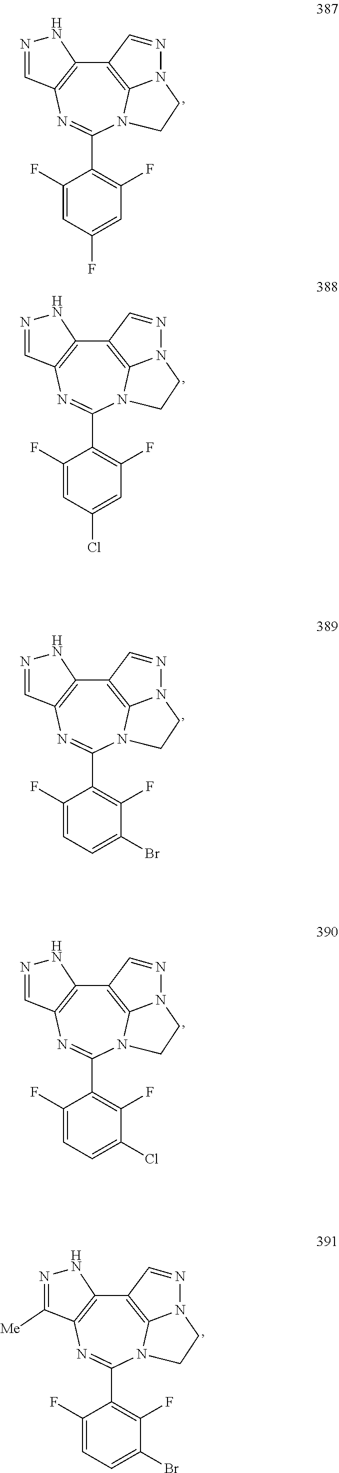 [SMILES: Fc1cc(F)c(C2=Nc3cn[nH]c3-c3cnn4c3N2CC4)c(F)c1 ; Fc1cc(Cl)cc(F)c1C1=Nc2cn[nH]c2-c2cnn3c2N1CC3 ; Fc1ccc(Br)c(F)c1C1=Nc2cn[nH]c2-c2cnn3c2N1CC3 ; Fc1ccc(Cl)c(F)c1C1=Nc2cn[nH]c2-c2cnn3c2N1CC3 ; Cc1n[nH]c2c1N=C(c1c(F)ccc(Br)c1F)N1CCn3ncc-2c31] 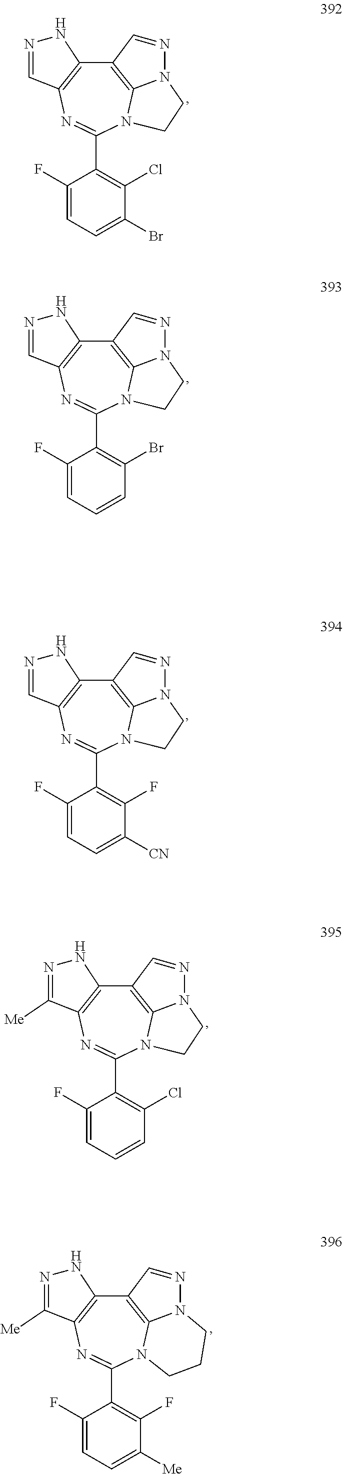 [SMILES: Fc1ccc(Br)c(Cl)c1C1=Nc2cn[nH]c2-c2cnn3c2N1CC3 ; Fc1cccc(Br)c1C1=Nc2cn[nH]c2-c2cnn3c2N1CC3 ; N#Cc1ccc(F)c(C2=Nc3cn[nH]c3-c3cnn4c3N2CC4)c1F ; Cc1n[nH]c2c1N=C(c1c(F)cccc1Cl)N1CCn3ncc-2c31 ; Cc1ccc(F)c(C2=Nc3c(C)n[nH]c3-c3cnn4c3N2CCC4)c1F] 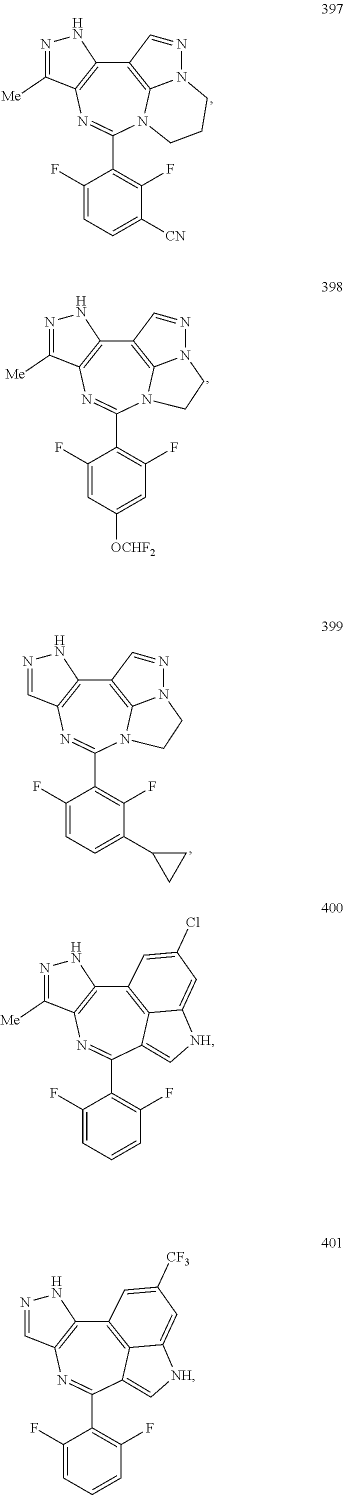 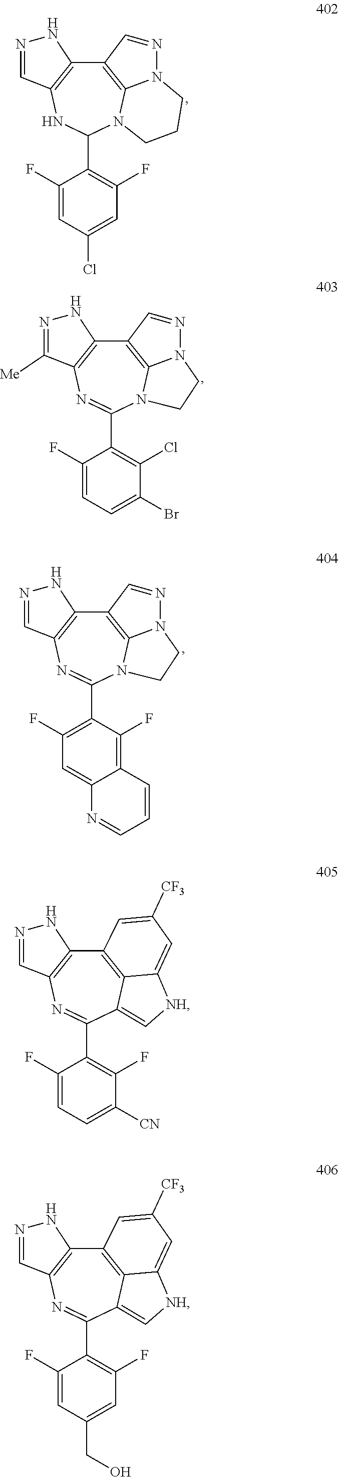 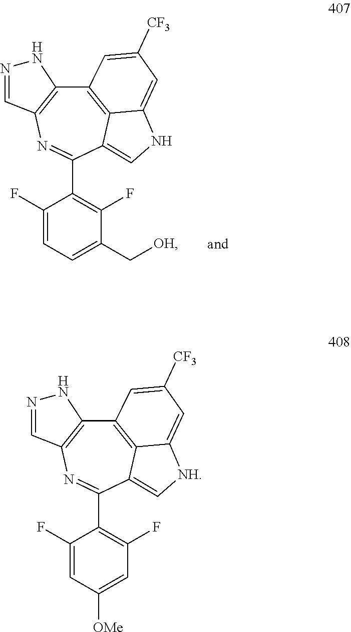 [SMILES: OCc1ccc(F)c(C2=Nc3cn[nH]c3-c3cc(C(F)(F)F)cc4[nH]cc2c34)c1F ; "and" ; COc1cc(F)c(C2=Nc3cn[nH]c3-c3cc(C(F)(F)F)cc4[nH]cc2c34)c(F)c1]
